(19) United States
(12) Patent Application Publication
(10) Pub. No.: US 2022/0243278 A1
(43) Pub. Date: Aug. 4, 2022

(54) DETECTING THE PRESENCE OR ABSENCE OF MULTIPLE TYPES OF CANCER

(71) Applicants: Mayo Foundation for Medical Education and Research, Rochester, MN (US); Exact Sciences Corporation, Madison, WI (US)

(72) Inventors: William R. Taylor, Lake City, MN (US); Hatim T. Allawi, Middleton, WI (US); Viatcheslav E. Katerov, Madison, WI (US); John B. Kisiel, Rochester, MN (US); Douglas W. Mahoney, Rochester, MN (US); David A. Ahlquist, Rochester, MN (US)

(21) Appl. No.: 17/587,963

(22) Filed: Jan. 28, 2022

Related U.S. Application Data
(60) Provisional application No. 63/278,889, filed on Nov. 12, 2021.
(60) Provisional application No. 63/143,611, filed on Jan. 29, 2021.

(51) Int. Cl. C12Q 1/6886; C12Q 1/6806; G01N 33/574

(52) U.S. Cl. CPC C12Q 1/6886; C12Q 1/6806; C12Q 2600/16; C12Q 2600/154; G01N 33/57488

(57) ABSTRACT

Provided herein is technology for screening multiple types of cancer from a biological sample, and particularly, but not exclusively, to methods, compositions, and related uses for simultaneously detecting the presence of multiple types of cancer (e.g., liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer) from a biological sample (e.g., stool sample, tissue sample, organ secretion sample, CSF sample, saliva sample, blood sample, plasma sample or urine sample).

### CROSS-REFERENCE TO RELATED APPLICATIONS

The present application claims priority to U.S. Provisional Patent Application No. 63/143,611, filed Jan. 29, 2021 and U.S. Provisional Patent Application No. 63/278,889, filed Nov. 12, 2021, which are hereby incorporated by reference in their entireties.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 53,000 Byte ASCII (Text) file named “39039-203_ST25” created on Jan. 28, 2022.

### FIELD OF INVENTION

Provided herein is technology for screening multiple types of cancer from a biological sample. In particular, the provided is related to methods, compositions, and related uses for simultaneously detecting the presence of multiple types of cancer (e.g., liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer) from a biological sample (e.g., stool sample, tissue sample, organ secretion sample, CSF sample, saliva sample, blood sample, plasma sample or urine sample).

### BACKGROUND

All too often, healthcare professionals can only make a cancer diagnosis after symptoms have developed—at which point it may be too late for curative treatment. Screening programs, such as Pap-smears for cervical cancer and mammograms for breast cancer, intend to overcome this problem by detecting cancer at an earlier stage. However, such tests are typically only available to a subset of the population (those at highest risk), are limited to a small number of cancers, and have variable rates of compliance. These methods can also be invasive or uncomfortable, which may discourage participation.

As such, there is an urgent need for improved diagnostic tools for detecting multiple types of cancer from a single biological sample.

The present invention addresses this need.

### SUMMARY

Provided herein is technology for screening multiple types of cancer from a biological sample, and particularly, but not exclusively, to methods, compositions, and related uses for simultaneously detecting the presence of multiple types of cancer (e.g., liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer) from a biological sample (e.g., stool sample, tissue sample, organ secretion sample, CSF sample, saliva sample, blood sample, plasma sample or urine sample).

Indeed, as described in Example I, experiments conducted during the course for identifying embodiments for the present invention identified a set of methylated DNA markers (MDMs), a set of protein markers, and a combination of MDMs and protein markers for simultaneously detecting the presence of multiple types of cancer (e.g., liver cancer, esophageal cancer, lung cancer, ovarian cancer, pancreatic cancer, gastric cancer, bladder cancer, breast cancer, cervical cancer, colorectal cancer, prostate cancer, renal cancer, uterine cancer) from a biological sample (e.g., stool sample, tissue sample, organ secretion sample, CSF sample, saliva sample, blood sample, plasma sample or urine sample).

In particular, such experiments identified the following combination of MDMs and protein markers and/or panel of MDMs and protein markers for detecting multiple types of cancer (e.g., liver cancer, esophageal cancer, lung cancer, ovarian cancer, pancreatic cancer, gastric cancer, bladder cancer, breast cancer, cervical cancer, colorectal cancer, prostate cancer, renal cancer, uterine cancer) from a biological sample (e.g., stool sample, tissue sample, organ secretion sample, CSF sample, saliva sample, blood sample, plasma sample or urine sample):
Methylated DNA Markers: FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, ST8SIA1, IFFO1, HOXA9, HOPX, OSR2, QKI, RYR2, GPRIN1, ZNF569, CD1D, NTRK3, VAV3, and FAM59B (see, FIG. 3, Tables, 5, 13 and 14, Example I);
Methylated DNA Markers: FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, and ST8SIA1 (see, FIG. 3, Example I);
Methylated DNA Markers: GRIN2D, SHOX2, ZNF671, SIM2, TRH, CAPN2, CHST2_7890, FER1L4, FAIM2, PPP2R5C, TSPYL5, NDRG4, ZNF781, IFFO1, HOXA9, and HOPX (see, Table 5, Example I);
Methylated DNA Markers: GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B (see, Tables 13 and 14, Example I);
Methylated DNA Markers: FAIM2, CHST2, ZNF671, GRIN2D, CDO1 (see, FIG. 3, and Example I);
Methylated DNA Markers: ZNF671, GRIN2D, NDGR4, SHOX2, B3GALT6 (see, Example I);
Methylated DNA Markers: CDO1, GRIN2D, SHOX2, OSR2, QKI, SIM2, TRH, CAPN2, SFMBT2, CHST2, ST8SIA1, HOXA1, FER1L4, FAIM2, IFFO1, EMX1, ZNF671, PRKCB, HOXB2, BARX1, PPP2R5C, and TSPYL5 (see, Example I);
Protein Markers: CEA, CA125, CA19.9, AFP, and CA-15-3 (see, Example I);
Protein Markers: CEA, CA125, and CA19.9 (see, FIG. 2, and Example I); and
Protein Markers: CEA, CA125, CA19.9, and AFP (see, FIG. 4, and Example I).

As described herein, the technology provides a set of methylated DNA markers (MDMs) and subsets thereof (e.g., sets of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38), a set of protein markers (e.g., sets of 2, 3, 4, 5), and a combination of MDMs and protein markers for simultaneously detecting the presence of multiple types of cancer from a biological sample (e.g., stool sample, tissue sample, organ secretion sample, CSF sample, saliva sample, blood sample, plasma sample or urine sample).

In certain embodiments, methods for characterizing a biological sample, for instance, a biological sample from a human subject, are provided comprising one or both of:

a) measuring a methylation level of one or more methylated markers selected from FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, ST8SIA1, IFFO1, HOXA9, HOPX, OSR2, QKI, RYR2, GPRIN1, ZNF569, CD1D, NTRK3, VAV3, and FAM59B in the biological sample; and

b) measuring an expression and/or activity level of one or more protein markers selected from CEA, CA125, CA19.9, AFP, and CA-15-3 in the biological sample.

In some embodiments wherein if a methylation level of one or more methylated markers is measured, then the measured methylation level of the one or more methylation markers is compared to a methylation level of a corresponding one or more methylation markers in control samples without a specific type of cancer; and/or

wherein if an expression and/or activity level of one or more protein markers is measured, then the measured expression and/or activity level of the one or more protein markers is compared to an expression and/or activity level of a corresponding one or more protein markers in control samples without a specific type of cancer.

In some embodiments, the method further comprises determining that the human subject has more than one type of cancer when one or both of:

i) the methylation level measured in the one or more methylation markers is higher than the methylation level measured in the respective control samples; and

ii) the expression and/or activity level of one or more protein markers is higher than the expression and/or activity level measured in the respective control samples.

In some embodiments, the more than one type of cancer is any type of cancer. In some embodiments, the more than one type of cancer is selected from liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer.

In some embodiments, measuring a methylation level of one or more methylated markers comprises treating DNA from the biological sample with a bisulfite-free and base-resolution sequencing method for direct detection of 5-methylcytosine and 5-hydroxymethylcytosine.

In some embodiments, measuring a methylation level of one or more methylated markers comprises treating DNA from the biological sample with a reagent that modifies DNA in a methylation-specific manner. In some embodiments, the reagent that modifies DNA in a methylation-specific manner is a borane reducing agent, for instance the borane reducing agent may be a 2-picoline borane. In some embodiments, the reagent comprises one or more of a methylation-sensitive restriction enzyme, a methylation-dependent restriction enzyme, and a bisulfite reagent. In some embodiments, the reagent is a bisulfite reagent, and the treating produces bisulfite-treated DNA.

In some embodiments, the treated DNA is amplified with a set of primers specific for the one or more methylated markers. In some embodiments, the set of primers for each of the selected one or more methylated markers is selected from the group recited in Table 2. In some embodiments, the set of primers specific for each the selected one or more methylated markers is capable of binding an amplicon bound by a primer sequence for the specific methylated marker gene recited in Table 2, wherein the amplicon bound by the primer sequence for the methylated marker gene recited in Table 2 is at least a portion of a genetic region for the methylated marker recited in Table 1. In some embodiments, the set of primers specific for each the selected one or more methylated markers is a set of primers that specifically binds at least a portion of a genetic region comprising chromosomal coordinates for a methylated marker recited in Table 1.

In some embodiments, measuring a methylation level of one or more methylated markers comprises multiplex amplification.

In some embodiments, measuring a methylation level of one or more methylated markers comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

In some embodiments, measuring a methylation level of one or more methylated markers comprises measuring methylation of a CpG site for each of the one or more methylation markers. In some embodiments, the CpG site is present in a coding region or a regulatory region.

In some embodiments, the one or more methylated markers is described by the genomic coordinates shown in Table 1.

In some embodiments, the biological sample is a stool sample, a tissue sample, an organ secretion sample, a CSF sample, a saliva sample, a blood sample, a plasma sample, or a urine sample.

In some embodiments, the human subject has or is suspected of having cancer.

In some embodiments, the one or more methylated markers are selected from one of the following groups:

FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, and ST8SIA1;

GRIN2D, SHOX2, ZNF671, SIM2, TRH, CAPN2, CHST2_7890, FER1L4, FAIM2, PPP2R5C, TSPYL5, NDRG4, ZNF781, IFFO1, HOXA9, and HOPX;

GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B;

CDO1, GRIN2D, SHOX2, OSR2, QKI, SIM2, TRH, CAPN2, SFMBT2, CHST2, ST8SIA1, HOXA1, FER1L4, FAIM2, IFFO1, EMX1, ZNF671, PRKCB, HOXB2, BARX1, PPP2R5C, and TSPYL5;

ZNF671, GRIN2D, NDGR4, SHOX2, B3GALT6; and

FAIM2, CHST2, ZNF671, GRIN2D, CDO1.

In certain embodiments, methods for preparing a deoxyribonucleic acid (DNA) fraction from a biological sample useful for analyzing one or more genetic loci involved in one or more chromosomal aberrations are provided, comprising:
(a) extracting genomic DNA from a biological sample;
(b) producing a fraction of the extracted genomic DNA by:
   (i) treating the extracted genomic DNA;
   (ii) amplifying the treated genomic DNA using separate primers specific for one or more of the following methylation markers: FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, ST8SIA1, IFFO1, HOXA9, HOPX, OSR2, QKI, RYR2, GPRIN1, ZNF569, CD1D, NTRK3, VAV3, and FAM59B;
(c) analyzing one or more genetic loci in the produced fraction of the extracted genomic DNA by measuring a methylation level for each of the one or more methylation markers.

In some embodiments, treating the extracted genomic DNA comprises treating the extracted genomic DNA with a reagent that modifies DNA in a methylation-specific manner. In some embodiments, the reagent that modifies DNA in a methylation-specific manner is a borane reducing agent, for instance the borane reducing agent may be 2-picoline borane In some embodiments, the reagent comprises one or more of a methylation-sensitive restriction enzyme, a methylation-dependent restriction enzyme, and a bisulfite reagent. In some embodiments, the reagent is a bisulfite reagent, and the treating produces bisulfite-treated DNA.

In some embodiments, the set of primers specific for the one or more methylated markers is selected from the group recited in Table 2. In some embodiments, the set of primers specific for each the selected one or more methylated markers is capable of binding an amplicon bound by a primer sequence for the specific methylated marker gene recited in Table 2, the amplicon bound by the primer sequence for the methylated marker gene recited in Table 2 is at least a portion of a genetic region for the methylated marker recited in Table 1. In some embodiments, the set of primers specific for each the selected one or more methylated markers is a set of primers that specifically binds at least a portion of a genetic region comprising chromosomal coordinates for the specific methylated marker recited in Table 1.

In some embodiments, measuring a methylation level of one or more methylated markers comprises multiplex amplification.

In some embodiments, measuring a methylation level of one or more methylated markers comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

In some embodiments, measuring a methylation level of one or more methylated markers comprises measuring methylation of a CpG site for the one or more methylation markers. In some embodiments, the CpG site is present in a coding region or a regulatory region.

In some embodiments, the one or more methylated markers is described by the genomic coordinates shown in Table 1.

In some embodiments, the biological sample is a stool sample, a tissue sample, an organ secretion sample, a CSF sample, a saliva sample, a blood sample, a plasma sample, or a urine sample.

In some embodiments, the biological sample is from a human subject. In some embodiments, the human subject has or is suspected of having cancer.

In some embodiments, the one or more methylated markers are selected from one of the following groups:

FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, and ST8SIA1;

GRIN2D, SHOX2, ZNF671, SIM2, TRH, CAPN2, CHST2_7890, FER1L4, FAIM2, PPP2R5C, TSPYL5, NDRG4, ZNF781, IFFO1, HOXA9, and HOPX;

GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B;

CDO1, GRIN2D, SHOX2, OSR2, QKI, SIM2, TRH, CAPN2, SFMBT2, CHST2, ST8SIA1, HOXA1, FER1L4, FAIM2, IFFO1, EMX1, ZNF671, PRKCB, HOXB2, BARX1, PPP2R5C, and TSPYL5;

ZNF671, GRIN2D, NDGR4, SHOX2, B3GALT6; and

FAIM2, CHST2, ZNF671, GRIN2D, CDO1.

In some embodiments, each of the analyzed one or more genetic loci is associated with any type of cancer. In some embodiments, each of the analyzed one or more genetic loci is associated with two or more types of cancer. In some embodiments, each of the analyzed one or more genetic loci is associated with one or more of liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer.

In certain embodiments, the technology is related to assessing the presence of and methylation state of one or more of the methylated markers described herein in a biological sample (e.g., stool sample, tissue sample, organ secretion sample, CSF sample, saliva sample, blood sample, plasma sample or urine sample). These methylated markers comprise one or more differentially methylated regions (DMR) as discussed herein, e.g., as provided in Table 1 and FIG. 1. Methylation state is assessed in embodiments of the technology. As such, the technology provided herein is not restricted in the method by which a gene's methylation state is measured and thus the methylation state of a gene may be measured by any method know in the art.

In some embodiments, the plurality of different target regions comprise a reference target region, and in certain preferred embodiments, the reference target region comprises (3-actin and/or ZDHHC1, and/or B3GALT6.

Also provided herein are compositions and kits for practicing any of the methods described herein. For example, in some embodiments, reagents (e.g., primers, probes) specific for one or more methylated markers and/or protein markers are provided alone or in sets (e.g., sets of primers pairs for amplifying a plurality of markers). Additional reagents for conducting a detection assay may also be provided (e.g., enzymes, buffers, positive and negative controls for conducting QuARTS, PCR, sequencing, bisulfite, Ten-Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), a borane reducing agent, or other assays). In some embodiments, the kits contain a reagent capable of modifying DNA in a methylation-specific manner (e.g., a methylation-sensitive restriction enzyme, a methylation-dependent restriction enzyme, and a bisulfite reagent) (e.g., a methylation-sensitive restriction enzyme, a methylation-dependent restriction enzyme, Ten-Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), borane reducing agent), and/or an agent capable of detecting an expression or activity level of a protein marker described herein. In some embodiments, the kits containing one or more reagents necessary, sufficient, or useful for conducting a method are provided. Also provided are reactions mixtures containing the reagents. Further provided are master mix reagent sets containing a plurality of reagents that may be added to each other and/or to a test sample to complete a reaction mixture. In some embodiments, the kit comprises a control nucleic acid comprising one or more sequences from DMR 1-38 (from Table 1) and having a methylation state associated with a subject who has a specific type of cancer. In some embodiments, the kit comprises a sample collector for obtaining a sample from a subject (e.g., a stool sample; tissue sample; plasma sample, serum sample, whole blood sample). In some embodiments, the kit comprises an oligonucleotide as described herein.

Provided herein are methods and materials for detecting the presence of one or more methylated markers and/or the presence of aneuploidy in a biological sample obtained from a subject. In some embodiments, the presence of one or methylated markers and/or the presence of aneuploidy are tested simultaneously (e.g., in one testing procedure, including embodiments in which the testing procedure itself may include multiple discrete test methods of systems). In some embodiments, the presence of one or more methylated markers of one or more classes of biomarkers and/or the presence of aneuploidy are tested sequentially (e.g., in two or more different testing procedures conducted at two or more different time points, including embodiments in which the testing procedure itself may include multiple discrete test methods of systems). In some embodiments of both simultaneous and sequential testing for the presence of one or more methylated markers and/or the presence of aneuploidy, the testing may be performed on a single sample or may be performed on two or more different samples (e.g., two or more different samples obtained from the same subject).

### BRIEF DESCRIPTION OF THE DRAWINGS

FIG. 1: Marker chromosomal regions used for various methylated DNA markers recited in Table 1 and related primer and probe information. Shown are naturally occurring sequences (WT) and bisulfite-modified sequences (BST) from PCR target regions.

FIG. 2: A combination of 3 proteins (CEA, CA125, CA19-9) and 5 MDMs (ZNF671, GRIN2D, NDGR4, SHOX2, B3GALT6) resulted in an area under the receiver operating characteristics curve (AUC) of 0.95 and an overall sensitivity of 87% for all cancers at 95% specificity.

FIG. 3: A combination of 5 MDMs (FAIM2, CHST2, ZNF671, GRIN2D, CDO1) resulted in an overall sensitivity of 74% for all cancers at 94% specificity.

FIG. 4: A combination of 4 proteins (CEA, CA125, CA19.9, AFP) resulted in an overall sensitivity of 62% for all cancers at 96% specificity.

### DEFINITIONS

To facilitate an understanding of the present technology, a number of terms and phrases are defined below. Additional definitions are set forth throughout the detailed description.

Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or” unless the context clearly dictates otherwise. The term “based on” is not exclusive and allows for being based on additional factors not described, unless the context clearly dictates otherwise. In addition, throughout the specification, the meaning of “a”, “an”, and “the” include plural references. The meaning of “in” includes “in” and “on.”

The transitional phrase “consisting essentially of” as used in claims in the present application limits the scope of a claim to the specified materials or steps “and those that do not materially affect the basic and novel characteristic(s)” of the claimed invention, as discussed in In re Herz, 537 F.2d 549, 551-52, 190 USPQ 461, 463 (CCPR 1976). For example, a composition “consisting essentially of” recited elements may contain an unrecited contaminant at a level such that, though present, the contaminant does not alter the function of the recited composition as compared to a pure composition, i.e., a composition “consisting of” the recited components.

The term “one or more”, as used herein, refers to a number higher than one. For example, the term “one or more” encompasses any of the following: two or more, three or more, four or more, five or more, six or more, seven or more, eight or more, nine or more, ten or more, twelve or more, thirteen or more, fourteen or more, fifteen or more, twenty or more, fifty or more, 100 or more, or an even greater number.

The term “one or more but less than a higher number”, “two or more but less than a higher number”, “three or more but less than a higher number”, “four or more but less than a higher number”, “five or more but less than a higher number”, “six or more but less than a higher number”, “seven or more but less than a higher number”, “eight or more but less than a higher number”, “nine or more but less than a higher number”, “ten or more but less than a higher number”, “eleven or more but less than a higher number”, “twelve or more but less than a higher number”, “thirteen or more but less than a higher number”, “fourteen or more but less than a higher number”, or “fifteen or more but less than a higher number” is not limited to a higher number. For example, the higher number can be 10,000, 1,000, 100, 50, etc. For example, the higher number can be approximately 50 (e.g., 50, 49, 48, 47, 46, 45, 44, 43, 42, 41, 40, 39, 38, 37, 36, 35, 34, 33, 32, 31, 32, 30, 29, 28, 27, 26, 25, 24, 23, 22, 21, 20, 19, 18, 17, 16, 15, 14, 13, 12, 11, 10, 9, 8, 7, 6, 5, 4, 3 or 2).

The term “one or more methylated markers” or “one or more DMRs” or “one or more genes” or “one or more markers” or “a plurality of methylated markers” or “a plurality of markers” or “a plurality of genes” or “a plurality of DMRs” is similarly not limited to a particular numerical combination. Indeed, any numerical combination of methylated markers is contemplated (e.g., 1-2 methylated markers, 1-3, 1-4, 1-5. 1-6, 1-7, 1-8, 1-9, 1-10, 1-11, 1-12, 1-13, 1-14, 1-15, 1-16, 1-17, 1-18, 1-19, 1-20, 1-21, 1-22, 1-23, 1-24, 1-25, 1-26, 1-27, 1-28, 1-29, 1-30, 1-31, 1-32, 1-33, 1-34, 1-35, 1-36, 1-37, 1-38) (e.g., 2-3, 2-4, 2-5, 2-6, 2-7, 2-8, 2-9, 2-10, 2-11, 2-12, 2-13, 2-14, 2-15, 2-16, 2-17, 2-18, 2-19, 2-20, 2-21, 2-22, 2-23, 2-24, 2-25, 2-26, 2-27, 2-28, 2-29, 2-30, 2-31, 2-32, 2-33, 2-34, 2-35, 2-36, 2-37, 2-38) (e.g., 3-4, 3-5, 3-6, 3-7, 3-8, 3-9, 3-10, 3-11, 3-12, 3-13, 3-14, 3-15, 3-16, 3-17, 3-18, 3-19, 3-20, 3-21, 3-22, 3-23, 3-24, 3-25, 3-26, 3-27, 3-28, 3-29, 3-30, 3-31, 3-32, 3-33, 3-34, 3-35, 3-36, 3-37, 3-38) (e.g., 4-5, 4-6, 4-7, 4-8, 4-9, 4-10, 4-11, 4-12, 4-13, 4-14, 4-15, 4-16, 4-17, 4-18, 4-19, 4-20, 4-21, 4-22, 4-23, 4-24, 4-25, 4-26, 4-27, 4-28, 4-29, 4-30, 4-31, 4-32, 4-33, 4-34, 4-35, 4-36, 4-37, 4-38) (e.g., 5-6, 5-7, 5-8, 5-9, 5-10, 5-11, 5-12, 5-13, 5-14, 5-15, 5-16, 5-17, 5-18, 5-19, 5-20, 5-21, 5-22, 5-23, 5-24, 5-25, 5-26, 5-27, 5-28, 5-29, 5-30, 5-31, 5-32, 5-33, 5-34, 5-35, 5-36, 5-37, 5-38) (e.g., 6-7, 6-8, 6-9, 6-10, 6-11, 6-12, 6-13, 6-14, 6-15, 6-16, 6-17, 6-18, 6-19, 6-20, 6-21, 6-22, 6-23, 6-24, 6-25, 6-26, 6-27, 6-28, 6-29, 6-30, 6-31, 6-32, 6-33, 6-34, 6-35, 6-36, 6-37, 6-38) (e.g., 7-8, 7-9, 7-10, 7-11, 7-12, 7-13, 7-14, 7-15, 7-16, 7-17, 7-18, 7-19, 7-20, 7-21, 7-22, 7-23, 7-24, 7-25, 7-26, 7-27, 7-28, 7-29, 7-30, 7-31, 7-32, 7-33, 7-34, 7-35, 7-36, 7-37, 7-38) (e.g., 8-9, 8-10, 8-11, 8-12, 8-13, 8-14, 8-15, 8-16, 8-17, 8-18, 8-19, 8-20, 8-21, 8-22, 8-23, 8-24, 8-25, 8-26, 8-27, 8-28, 8-29, 8-30, 8-31, 8-32, 8-33, 8-34, 8-35, 8-36, 8-37, 8-38) (e.g., 9-10, 9-11, 9-12, 9-13, 9-14, 9-15, 9-16, 9-17, 9-18, 9-19, 9-20, 9-21, 9-22, 9-23, 9-24, 9-25, 9-26, 9-27, 9-28, 9-29, 9-30, 9-31, 9-32, 9-33, 9-34, 9-35, 9-36, 9-37, 9-38) (e.g., 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 10-31, 10-32, 10-33, 10-34, 10-35, 10-36, 10-37, 10-38) (e.g., 11-12, 11-13, 11-14, 11-15, 11-16, 11-17, 11-18, 11-19, 11-20, 11-21, 11-22, 11-23, 11-24, 11-25, 11-26, 11-27, 11-28, 11-29, 11-30, 11-31, 11-32, 11-33, 11-34, 11-35, 11-36, 11-37, 11-38) (e.g., 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 12-31, 12-32, 12-33, 12-34, 12-35, 12-36, 12-37, 12-38) (e.g., 13-14, 13-15, 13-16, 13-17, 13-18, 13-19, 13-20, 13-21, 13-22, 13-23, 13-24, 13-25, 13-26, 13-27, 13-28, 13-29, 13-30, 13-31, 13-32, 13-33, 13-34, 13-35, 13-36, 13-37, 13-38) (e.g., 14-15, 14-16, 14-17, 14-18, 14-19, 14-20, 14-21, 14-22, 14-23, 14-24, 14-25, 14-26, 14-27, 14-28, 14-29, 14-30, 14-31, 14-32, 14-33, 14-34, 14-35, 14-36, 14-37, 14-38) (e.g., 15-16, 15-17, 15-18, 15-19, 15-20, 15-21, 15-22, 15-23, 15-24, 15-25, 15-26, 15-27, 15-28, 15-29, 15-30, 15-31, 15-32, 15-33, 15-34, 15-35, 15-36, 15-37, 15-38) (e.g., 16-17, 16-18, 16-19, 16-20, 16-21, 16-22, 16-23, 16-24, 16-25, 16-26, 16-27, 16-28, 16-29, 16-30, 16-31, 16-32, 16-33, 16-34, 16-35, 16-36, 16-37, 16-38) (e.g., 17-18, 17-19, 17-20, 17-21, 17-22, 17-23, 17-24, 17-25, 17-26, 17-27, 17-28, 17-29, 17-30, 17-31, 17-32, 17-33, 17-34, 17-35, 17-36, 17-37, 17-38) (e.g., 18-19, 18-20, 18-21, 18-22, 18-23, 18-24, 18-25, 18-26, 18-27, 18-28, 18-29, 18-30, 18-31, 18-32, 18-33, 18-34, 18-35, 18-36, 18-37, 18-38) (e.g., 19-20, 19-21, 19-22, 19-23, 19-24, 19-25, 19-26, 19-27, 19-28, 19-29, 19-30, 19-31, 19-32, 19-33, 19-34, 19-35, 19-36, 19-37, 19-38) (e.g., 20-21, 20-22, 20-23, 20-24, 20-25, 20-26, 20-27, 20-28, 20-29, 20-30, 20-31, 20-32, 20-33, 20-34, 20-35, 20-36, 20-37, 20-38) (e.g., 21-22, 21-23, 21-24, 21-25, 21-26, 21-27, 21-28, 21-29, 21-30, 21-31, 21-32, 21-33, 21-34, 21-35, 21-36, 21-37, 21-38) (e.g., 22-23, 22-24, 22-25, 22-26, 22-27, 22-28, 22-29, 22-30, 22-31, 22-32, 22-33, 22-34, 22-35, 22-36, 22-37, 22-38) (e.g., 23-24, 23-25, 23-26, 23-27, 23-28, 23-29, 23-30, 23-31, 23-32, 23-33, 23-34, 23-35, 23-36, 23-37, 23-38) (e.g., 24-25, 24-26, 24-27, 24-28, 24-29, 24-30, 24-31, 24-32, 24-33, 24-34, 24-35, 24-36, 24-37, 24-38) (e.g., 25-26, 25-27, 25-28, 25-29, 25-30, 25-31, 25-32, 25-33, 25-34, 25-35, 25-36, 25-37, 25-38) (e.g., 26-27, 26-28, 26-29, 26-30, 26-31, 26-32, 26-33, 26-34, 26-35, 26-36, 26-37, 26-38) (e.g., 27-28, 27-29, 27-30, 27-31, 27-32, 27-33, 27-34, 27-35, 27-36, 27-37, 27-38) (e.g., 28-29, 28-30, 28-31, 28-32, 28-33, 28-34, 28-35, 28-36, 28-37, 28-38) (e.g., 29-30, 29-31, 29-32, 29-33, 29-34, 29-35, 29-36, 29-37, 29-38) (e.g., 30-31, 30-32, 30-33, 30-34, 30-35, 30-36, 30-37, 30-38) (e.g., 31-32, 31-33, 31-34, 31-35, 31-36, 31-37, 31-38) (e.g., 32-33, 32-34, 32-35, 32-36, 32-37, 32-38) (e.g., 33-34, 33-35, 33-36, 33-37, 33-38) (e.g., 34-35, 34-36, 34-37, 34-38) (e.g., 35-36, 35-37, 35-38) (e.g., 36-37, 36-38) (e.g., 37-38) (e.g., 38 or fewer; 37 or fewer; 36 or fewer; 35 or fewer; 34 or fewer; 33 or fewer; 32 or fewer; 31 or fewer; 30 or fewer; 29 or fewer; 28 or fewer; 27 or fewer; 26 or fewer; 25 or fewer; 24 or fewer; 23 or fewer; 22 or fewer; 21 or fewer; 20 or fewer; 19 or fewer; 18 or fewer; 17 or fewer; 16 or fewer; 15 or fewer; 14 or fewer; 13 or fewer; 12 or fewer; 11 or fewer; 10 or fewer; 9 or fewer; 8 or fewer; 7 or fewer; 6 or fewer; 5 or fewer; 4 or fewer; 3 or fewer; 2 or 1).

The term “one or more protein markers” is similarly not limited to a particular numerical combination. Indeed, any numerical combination of protein markers is contemplated (e.g., 1-2 protein markers, 1-3, 1-4, 1-5) (e.g., 2-3, 2-4, 2-5) (e.g., 3-4, 3-5) (e.g., 4-5) (e.g., 5 or fewer; 4 or fewer; 3 or fewer; 2 or 1).

The term “multiple types of cancer” or “one or more types of cancer” or “a plurality of different types of cancer” is similarly not limited to a particular numerical combination. Indeed, any numerical combination of types of cancer (e.g., liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer) is contemplated (e.g., 1-2 types of cancer, 1-3, 1-4, 1-5. 1-6, 1-7, 1-8, 1-9, 1-10, 1-11, 1-12, 1-13) (e.g., 13 or fewer; 12 or fewer; 11 or fewer; 10 or fewer; 9 or fewer; 8 or fewer; 7 or fewer; 6 or fewer; 5 or fewer; 4 or fewer; 3 or fewer; 2 or 1).

As used herein, a “nucleic acid” or “nucleic acid molecule” generally refers to any ribonucleic acid or deoxyribonucleic acid, which may be unmodified or modified DNA or RNA. “Nucleic acids” include, without limitation, single- and double-stranded nucleic acids. As used herein, the term “nucleic acid” also includes DNA as described above that contains one or more modified bases. Thus, DNA with a backbone modified for stability or for other reasons is a “nucleic acid”. The term “nucleic acid” as it is used herein embraces such chemically, enzymatically, or metabolically modified forms of nucleic acids, as well as the chemical forms of DNA characteristic of viruses and cells, including for example, simple and complex cells.

The terms “oligonucleotide” or “polynucleotide” or “nucleotide” or “nucleic acid” refer to a molecule having two or more deoxyribonucleotides or ribonucleotides, preferably more than three, and usually more than ten. The exact size will depend on many factors, which in turn depends on the ultimate function or use of the oligonucleotide. The oligonucleotide may be generated in any manner, including chemical synthesis, DNA replication, reverse transcription, or a combination thereof. Typical deoxyribonucleotides for DNA are thymine, adenine, cytosine, and guanine. Typical ribonucleotides for RNA are uracil, adenine, cytosine, and guanine.

As used herein, the terms “locus” or “region” of a nucleic acid refer to a subregion of a nucleic acid, e.g., a gene on a chromosome, a single nucleotide, a CpG island, etc.

The terms “complementary” and “complementarity” refer to nucleotides (e.g., 1 nucleotide) or polynucleotides (e.g., a sequence of nucleotides) related by the base-pairing rules. For example, the sequence 5′-A-G-T-3′ is complementary to the sequence 3′-T-C-A-5′. Complementarity may be “partial,” in which only some of the nucleic acids' bases are matched according to the base pairing rules. Or, there may be “complete” or “total” complementarity between the nucleic acids. The degree of complementarity between nucleic acid strands effects the efficiency and strength of hybridization between nucleic acid strands. This is of particular importance in amplification reactions and in detection methods that depend upon binding between nucleic acids.

The term “gene” refers to a nucleic acid (e.g., DNA or RNA) sequence that comprises coding sequences necessary for the production of an RNA, or of a polypeptide or its precursor. A functional polypeptide can be encoded by a full length coding sequence or by any portion of the coding sequence as long as the desired activity or functional properties (e.g., enzymatic activity, ligand binding, signal transduction, etc.) of the polypeptide are retained. The term “portion” when used in reference to a gene refers to fragments of that gene. The fragments may range in size from a few nucleotides to the entire gene sequence minus one nucleotide. Thus, “a nucleotide comprising at least a portion of a gene” may comprise fragments of the gene or the entire gene.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

In addition to containing introns, genomic forms of a gene may also include sequences located on both the 5′ and 3′ ends of the sequences that are present on the RNA transcript. These sequences are referred to as “flanking” sequences or regions (these flanking sequences are located 5′ or 3′ to the non-translated sequences present on the mRNA transcript). The 5′ flanking region may contain regulatory sequences such as promoters and enhancers that control or influence the transcription of the gene. The 3′ flanking region may contain sequences that direct the termination of transcription, posttranscriptional cleavage, and polyadenylation.

The term “wild-type” when made in reference to a gene refers to a gene that has the characteristics of a gene isolated from a naturally occurring source. The term “wild-type” when made in reference to a gene product refers to a gene product that has the characteristics of a gene product isolated from a naturally occurring source. The term “wild-type” when made in reference to a protein refers to a protein that has the characteristics of a naturally occurring protein. The term “naturally-occurring” as applied to an object refers to the fact that an object can be found in nature. For example, a polypeptide or polynucleotide sequence that is present in an organism (including viruses) that can be isolated from a source in nature and which has not been intentionally modified by the hand of a person in the laboratory is naturally-occurring. A wild-type gene is often that gene or allele that is most frequently observed in a population and is thus arbitrarily designated the “normal” or “wild-type” form of the gene. In contrast, the term “modified” or “mutant” when made in reference to a gene or to a gene product refers, respectively, to a gene or to a gene product that displays modifications in sequence and/or functional properties (e.g., altered characteristics) when compared to the wild-type gene or gene product. It is noted that naturally-occurring mutants can be isolated; these are identified by the fact that they have altered characteristics when compared to the wild-type gene or gene product.

The term “allele” refers to a variation of a gene; the variations include but are not limited to variants and mutants, polymorphic loci, and single nucleotide polymorphic loci, frameshift, and splice mutations. An allele may occur naturally in a population or it might arise during the lifetime of any particular individual of the population.

Thus, the terms “variant” and “mutant” when used in reference to a nucleotide sequence refer to a nucleic acid sequence that differs by one or more nucleotides from another, usually related, nucleotide acid sequence. A “variation” is a difference between two different nucleotide sequences; typically, one sequence is a reference sequence.

The term “primer” refers to an oligonucleotide, whether occurring naturally as, e.g., a nucleic acid fragment from a restriction digest, or produced synthetically, that is capable of acting as a point of initiation of synthesis when placed under conditions in which synthesis of a primer extension product that is complementary to a nucleic acid template strand is induced, (e.g., in the presence of nucleotides and an inducing agent such as a DNA polymerase, and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, and the use of the method. In some embodiments, the primer pair is specific for a specific MDM (e.g., MDMs in Table 1) and specifically binds at least a portion of a genetic region comprising the MDM (e.g., chromosomal coordinates in Table 1).

The term “probe” refers to an oligonucleotide (e.g., a sequence of nucleotides), whether occurring naturally as in a purified restriction digest or produced synthetically, recombinantly, or by PCR amplification, that is capable of hybridizing to another oligonucleotide of interest. A probe may be single-stranded or double-stranded. Probes are useful in the detection, identification, and isolation of particular gene sequences (e.g., a “capture probe”). It is contemplated that any probe used in the present invention may, in some embodiments, be labeled with any “reporter molecule,” so that is detectable in any detection system, including, but not limited to enzyme (e.g., ELISA, as well as enzyme-based histochemical assays), fluorescent, radioactive, and luminescent systems. It is not intended that the present invention be limited to any particular detection system or label.

The term “target,” as used herein refers to a nucleic acid sought to be sorted out from other nucleic acids, e.g., by probe binding, amplification, isolation, capture, etc. For example, when used in reference to the polymerase chain reaction, “target” refers to the region of nucleic acid bounded by the primers used for polymerase chain reaction, while when used in an assay in which target DNA is not amplified, e.g., in some embodiments of an invasive cleavage assay, a target comprises the site at which a probe and invasive oligonucleotides (e.g., INVADER oligonucleotide) bind to form an invasive cleavage structure, such that the presence of the target nucleic acid can be detected. A “segment” is defined as a region of nucleic acid within the target sequence.

Accordingly, as used herein, “non-target”, e.g., as it is used to describe a nucleic acid such as a DNA, refers to nucleic acid that may be present in a reaction, but that is not the subject of detection or characterization by the reaction. In some embodiments, non-target nucleic acid may refer to nucleic acid present in a sample that does not, e.g., contain a target sequence, while in some embodiments, non-target may refer to exogenous nucleic acid, i.e., nucleic acid that does not originate from a sample containing or suspected of containing a target nucleic acid, and that is added to a reaction, e.g., to normalize the activity of an enzyme (e.g., polymerase) to reduce variability in the performance of the enzyme in the reaction.

As used herein, “methylation” refers to cytosine methylation at positions C5 or N4 of cytosine, the N6 position of adenine, or other types of nucleic acid methylation. In vitro amplified DNA is usually unmethylated because typical in vitro DNA amplification methods do not retain the methylation pattern of the amplification template. However, “unmethylated DNA” or “methylated DNA” can also refer to amplified DNA whose original template was unmethylated or methylated, respectively.

As used herein, the term “amplification reagents” refers to those reagents (deoxyribonucleoside triphosphates, buffer, etc.), needed for amplification except for primers, nucleic acid template, and the amplification enzyme. Typically, amplification reagents along with other reaction components are placed and contained in a reaction vessel.

As used herein, the term “control” when used in reference to nucleic acid detection or analysis refers to a nucleic acid having known features (e.g., known sequence, known copy-number per cell), for use in comparison to an experimental target (e.g., a nucleic acid of unknown concentration). A control may be an endogenous, preferably invariant gene against which a test or target nucleic acid in an assay can be normalized. Such normalizing controls for sample-to-sample variations that may occur in, for example, sample processing, assay efficiency, etc., and allows accurate sample-to-sample data comparison. Genes that find use for normalizing nucleic acid detection assays on human samples include, e.g., β-actin, ZDHHC1, and B3GALT6 (see, e.g., U.S. patent application Ser. Nos 14/966,617 and 62/364,082, each incorporated herein by reference). As used herein “ZDHHC1” refers to a gene encoding a protein characterized as a zinc finger, DHHC-type containing 1, located in human DNA on Chr 16 (16q22.1) and belonging to the DHHC palmitoyltransferase family.

Controls may also be external. For example, in quantitative assays such as qPCR, QuARTS, etc., a “calibrator” or “calibration control” is a nucleic acid of known sequence, e.g., having the same sequence as a portion of an experimental target nucleic acid, and a known concentration or series of concentrations (e.g., a serially diluted control target for generation of calibration curved in quantitative PCR). Typically, calibration controls are analyzed using the same reagents and reaction conditions as are used on an experimental DNA. In certain embodiments, the measurement of the calibrators is done at the same time, e.g., in the same thermal cycler, as the experimental assay. In preferred embodiments, multiple calibrators may be included in a single plasmid, such that the different calibrator sequences are easily provided in equimolar amounts. In particularly preferred embodiments, plasmid calibrators are digested, e.g., with one or more restriction enzymes, to release calibrator portion from the plasmid vector. See, e.g., WO 2015/066695, which is included herein by reference.

As used herein a “methylated nucleotide” or a “methylated nucleotide base” refers to the presence of a methyl moiety on a nucleotide base, where the methyl moiety is not present in a recognized typical nucleotide base. For example, cytosine does not contain a methyl moiety on its pyrimidine ring, but 5-methylcytosine contains a methyl moiety at position 5 of its pyrimidine ring. Therefore, cytosine is not a methylated nucleotide and 5-methylcytosine is a methylated nucleotide. In another example, thymine contains a methyl moiety at position 5 of its pyrimidine ring; however, for purposes herein, thymine is not considered a methylated nucleotide when present in DNA since thymine is a typical nucleotide base of DNA.

As used herein, a “methylated nucleic acid molecule” refers to a nucleic acid molecule that contains one or more methylated nucleotides.

As used herein, a “methylation state”, “methylation profile”, and “methylation status” of a nucleic acid molecule refers to the presence or absence of one or more methylated nucleotide bases in the nucleic acid molecule. For example, a nucleic acid molecule containing a methylated cytosine is considered methylated (e.g., the methylation state of the nucleic acid molecule is methylated). A nucleic acid molecule that does not contain any methylated nucleotides is considered unmethylated.

As used herein, the term “methylation level” as applied to a methylation marker refers to the amount of methylation within a particular methylation marker. Methylation level may also refer to the amount of methylation within a particular methylation marker in comparison with an established norm or control. Methylation level may also refer to whether one or more cytosine residues present in a CpG context have or do not have a methylation group. Methylation level may also refer to the fraction of cells in a sample that do or do not have a methylation group on such cytosines. Methylation level may also alternatively describe whether a single CpG di-nucleotide is methylated.

The methylation state of a particular nucleic acid sequence (e.g., a gene marker or DNA region as described herein) can indicate the methylation state of every base in the sequence or can indicate the methylation state of a subset of the bases (e.g., of one or more cytosines) within the sequence, or can indicate information regarding regional methylation density within the sequence with or without providing precise information of the locations within the sequence the methylation occurs.

The methylation state of a nucleotide locus in a nucleic acid molecule refers to the presence or absence of a methylated nucleotide at a particular locus in the nucleic acid molecule. For example, the methylation state of a cytosine at the 7th nucleotide in a nucleic acid molecule is methylated when the nucleotide present at the 7th nucleotide in the nucleic acid molecule is 5-methylcytosine. Similarly, the methylation state of a cytosine at the 7th nucleotide in a nucleic acid molecule is unmethylated when the nucleotide present at the 7th nucleotide in the nucleic acid molecule is cytosine (and not 5-methylcytosine).

The methylation status can optionally be represented or indicated by a “methylation value” (e.g., representing a methylation frequency, fraction, ratio, percent, etc.). A methylation value can be generated, for example, by quantifying the amount of intact nucleic acid present following restriction digestion with a methylation dependent restriction enzyme or by comparing amplification profiles after bisulfite reaction or by comparing sequences of bisulfite-treated and untreated nucleic acids or by comparing TET-treated and untreated nucleic acids. Accordingly, a value, e.g., a methylation value, represents the methylation status and can thus be used as a quantitative indicator of methylation status across multiple copies of a locus. This is of particular use when it is desirable to compare the methylation status of a sequence in a sample to a threshold or reference value.

As used herein, “methylation frequency” or “methylation percent (%)” refer to the number of instances in which a molecule or locus is methylated relative to the number of instances the molecule or locus is unmethylated.

The term “methylation score” as used herein is a score indicative of detected methylation events in a marker or panel of markers in comparison with median methylation events for the marker or panel of markers from a random population of mammals (e.g., a random population of 10, 20, 30, 40, 50, 100, or 500 mammals) that do not have a specific neoplasm of interest. An elevated methylation score in a marker or panel of markers can be any score provided that the score is greater than a corresponding reference score. For example, an elevated score of methylation in a marker or panel of markers can be 0.5, 1, 2, 3, 4, 5, 6, 7, 8, 9, 10, or more fold greater than the reference methylation score.

As such, the methylation state describes the state of methylation of a nucleic acid (e.g., a genomic sequence). In addition, the methylation state refers to the characteristics of a nucleic acid segment at a particular genomic locus relevant to methylation. Such characteristics include, but are not limited to, whether any of the cytosine (C) residues within this DNA sequence are methylated, the location of methylated C residue(s), the frequency or percentage of methylated C throughout any particular region of a nucleic acid, and allelic differences in methylation due to, e.g., difference in the origin of the alleles. The terms “methylation state”, “methylation profile”, and “methylation status” also refer to the relative concentration, absolute concentration, or pattern of methylated C or unmethylated C throughout any particular region of a nucleic acid in a biological sample. For example, if the cytosine (C) residue(s) within a nucleic acid sequence are methylated it may be referred to as “hypermethylated” or having “increased methylation”, whereas if the cytosine (C) residue(s) within a DNA sequence are not methylated it may be referred to as “hypomethylated” or having “decreased methylation”. Likewise, if the cytosine (C) residue(s) within a nucleic acid sequence are methylated as compared to another nucleic acid sequence (e.g., from a different region or from a different individual, etc.) that sequence is considered hypermethylated or having increased methylation compared to the other nucleic acid sequence. Alternatively, if the cytosine (C) residue(s) within a DNA sequence are not methylated as compared to another nucleic acid sequence (e.g., from a different region or from a different individual, etc.) that sequence is considered hypomethylated or having decreased methylation compared to the other nucleic acid sequence. Additionally, the term “methylation pattern” as used herein refers to the collective sites of methylated and unmethylated nucleotides over a region of a nucleic acid. Two nucleic acids may have the same or similar methylation frequency or methylation percent but have different methylation patterns when the number of methylated and unmethylated nucleotides are the same or similar throughout the region but the locations of methylated and unmethylated nucleotides are different. Sequences are said to be “differentially methylated” or as having a “difference in methylation” or having a “different methylation state” when they differ in the extent (e.g., one has increased or decreased methylation relative to the other), frequency, or pattern of methylation. The term “differential methylation” refers to a difference in the level or pattern of nucleic acid methylation in a cancer positive sample as compared with the level or pattern of nucleic acid methylation in a cancer negative sample. It may also refer to the difference in levels or patterns between patients that have recurrence of cancer after surgery versus patients who not have recurrence. Differential methylation and specific levels or patterns of DNA methylation are prognostic and predictive biomarkers, e.g., once the correct cut-off or predictive characteristics have been defined.

Methylation state frequency can be used to describe a population of individuals or a sample from a single individual. For example, a nucleotide locus having a methylation state frequency of 50% is methylated in 50% of instances and unmethylated in 50% of instances. Such a frequency can be used, for example, to describe the degree to which a nucleotide locus or nucleic acid region is methylated in a population of individuals or a collection of nucleic acids. Thus, when methylation in a first population or pool of nucleic acid molecules is different from methylation in a second population or pool of nucleic acid molecules, the methylation state frequency of the first population or pool will be different from the methylation state frequency of the second population or pool. Such a frequency also can be used, for example, to describe the degree to which a nucleotide locus or nucleic acid region is methylated in a single individual. For example, such a frequency can be used to describe the degree to which a group of cells from a tissue sample are methylated or unmethylated at a nucleotide locus or nucleic acid region.

Typically, methylation of human DNA occurs on a dinucleotide sequence including an adjacent guanine and cytosine where the cytosine is located 5′ of the guanine (also termed CpG dinucleotide sequences). Most cytosines within the CpG dinucleotides are methylated in the human genome, however some remain unmethylated in specific CpG dinucleotide rich genomic regions, known as CpG islands (see, e.g, Antequera et al. (1990) Cell 62: 503-514).

As used herein, a “CpG island” or “cytosine-phosphate-guanine island”) refers to a G: C-rich region of genomic DNA containing an increased number of CpG dinucleotides relative to total genomic DNA. A CpG island can be at least 100, 200, or more base pairs in length, where the G:C content of the region is at least 50% and the ratio of observed CpG frequency over expected frequency is 0.6; in some instances, a CpG island can be at least 500 base pairs in length, where the G:C content of the region is at least 55%) and the ratio of observed CpG frequency over expected frequency is 0.65. The observed CpG frequency over expected frequency can be calculated according to the method provided in Gardiner-Garden et al (1987) J. Mol. Biol. 196: 261-281. For example, the observed CpG frequency over expected frequency can be calculated according to the formula R=(A×B)/(C×D), where R is the ratio of observed CpG frequency over expected frequency, A is the number of CpG dinucleotides in an analyzed sequence, B is the total number of nucleotides in the analyzed sequence, C is the total number of C nucleotides in the analyzed sequence, and D is the total number of G nucleotides in the analyzed sequence. Methylation state is typically determined in CpG islands, e.g., at promoter regions. It will be appreciated though that other sequences in the human genome are prone to DNA methylation such as CpA and CpT (see Ramsahoye (2000) Proc. Natl. Acad. Sci. USA 97: 5237-5242; Salmon and Kaye (1970) Biochim. Biophys. Acta. 204: 340-351; Grafstrom (1985) Nucleic Acids Res. 13: 2827-2842; Nyce (1986) Nucleic Acids Res. 14: 4353-4367; Woodcock (1987) Biochem. Biophys. Res. Commun. 145: 888-894).

As used herein, a “methylation-specific reagent” refers to a reagent that modifies a nucleotide of the nucleic acid molecule as a function of the methylation state of the nucleic acid molecule, or a methylation-specific reagent, refers to a compound or composition or other agent that can change the nucleotide sequence of a nucleic acid molecule in a manner that reflects the methylation state of the nucleic acid molecule. Methods of treating a nucleic acid molecule with such a reagent can include contacting the nucleic acid molecule with the reagent, coupled with additional steps, if desired, to accomplish the desired change of nucleotide sequence. Such methods can be applied in a manner in which unmethylated nucleotides (e.g., each unmethylated cytosine) is modified to a different nucleotide. For example, in some embodiments, such a reagent can deaminate unmethylated cytosine nucleotides to produce deoxy uracil residues. Examples of such reagents include, but are not limited to, a methylation-sensitive restriction enzyme, a methylation-dependent restriction enzyme, a bisulfite reagent, a TET enzyme, and a borane reducing agent.

A change in the nucleic acid nucleotide sequence by a methylation-specific reagent can also result in a nucleic acid molecule in which each methylated nucleotide is modified to a different nucleotide.

The term “methylation assay” refers to any assay for determining the methylation state of one or more CpG dinucleotide sequences within a sequence of a nucleic acid.

The term “MS AP-PCR” (Methylation-Sensitive Arbitrarily-Primed Polymerase Chain Reaction) refers to the art-recognized technology that allows for a global scan of the genome using CG-rich primers to focus on the regions most likely to contain CpG dinucleotides, as described by Gonzalgo et al. (1997) Cancer Research 57: 594-599.

The term “MethyLight™” refers to the art-recognized fluorescence-based real-time PCR technique described by Eads et al. (1999) Cancer Res. 59: 2302-2306.

The term “HeavyMethyl™” refers to an assay wherein methylation specific blocking probes (also referred to herein as blockers) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers.

The term “Ms-SNuPE” (Methylation-sensitive Single Nucleotide Primer Extension) refers to the art-recognized assay described by Gonzalgo & Jones (1997) Nucleic Acids Res. 25: 2529-2531.

The term “MSP” (Methylation-specific PCR) refers to the art-recognized methylation assay described by Herman et al. (1996) Proc. Natl. Acad. Sci. USA 93: 9821-9826, and by U.S. Pat. No. 5,786,146.

The term “COBRA” (Combined Bisulfite Restriction Analysis) refers to the art-recognized methylation assay described by Xiong & Laird (1997) Nucleic Acids Res. 25: 2532-2534.

The term “MCA” (Methylated CpG Island Amplification) refers to the methylation assay described by Toyota et al. (1999) Cancer Res. 59: 2307-12, and in WO 00/26401A1.

As used herein, a “selected nucleotide” refers to one nucleotide of the four typically occurring nucleotides in a nucleic acid molecule (C, G, T, and A for DNA and C, G, U, and A for RNA), and can include methylated derivatives of the typically occurring nucleotides (e.g., when C is the selected nucleotide, both methylated and unmethylated C are included within the meaning of a selected nucleotide), whereas a methylated selected nucleotide refers specifically to a methylated typically occurring nucleotide and an unmethylated selected nucleotides refers specifically to an unmethylated typically occurring nucleotide.

The term “methylation-specific restriction enzyme” refers to a restriction enzyme that selectively digests a nucleic acid dependent on the methylation state of its recognition site. In the case of a restriction enzyme that specifically cuts if the recognition site is not methylated or is hemi-methylated (a methylation-sensitive enzyme), the cut will not take place (or will take place with a significantly reduced efficiency) if the recognition site is methylated on one or both strands. In the case of a restriction enzyme that specifically cuts only if the recognition site is methylated (a methylation-dependent enzyme), the cut will not take place (or will take place with a significantly reduced efficiency) if the recognition site is not methylated. Preferred are methylation-specific restriction enzymes, the recognition sequence of which contains a CG dinucleotide (for instance a recognition sequence such as CGCG or CCCGGG). Further preferred for some embodiments are restriction enzymes that do not cut if the cytosine in this dinucleotide is methylated at the carbon atom C5.

As used herein, the “sensitivity” of a given marker (or set of markers used together) refers to the percentage of samples that report a DNA methylation value above a threshold value that distinguishes between neoplastic and non-neoplastic samples. In some embodiments, a positive is defined as a histology-confirmed neoplasia that reports a DNA methylation value above a threshold value (e.g., the range associated with disease), and a false negative is defined as a histology-confirmed neoplasia that reports a DNA methylation value below the threshold value (e.g., the range associated with no disease). The value of sensitivity, therefore, reflects the probability that a DNA methylation measurement for a given marker obtained from a known diseased sample will be in the range of disease-associated measurements. As defined here, the clinical relevance of the calculated sensitivity value represents an estimation of the probability that a given marker would detect the presence of a clinical condition when applied to a subject with that condition.

As used herein, the “specificity” of a given marker (or set of markers used together) refers to the percentage of non-neoplastic samples that report a DNA methylation value below a threshold value that distinguishes between neoplastic and non-neoplastic samples. In some embodiments, a negative is defined as a histology-confirmed non-neoplastic sample that reports a DNA methylation value below the threshold value (e.g., the range associated with no disease) and a false positive is defined as a histology-confirmed non-neoplastic sample that reports a DNA methylation value above the threshold value (e.g., the range associated with disease). The value of specificity, therefore, reflects the probability that a DNA methylation measurement for a given marker obtained from a known non-neoplastic sample will be in the range of non-disease associated measurements. As defined here, the clinical relevance of the calculated specificity value represents an estimation of the probability that a given marker would detect the absence of a clinical condition when applied to a patient without that condition.

The term “AUC” as used herein is an abbreviation for the “area under a curve”. In particular it refers to the area under a Receiver Operating Characteristic (ROC) curve. The ROC curve is a plot of the true positive rate against the false positive rate for the different possible cut points of a diagnostic test. It shows the trade-off between sensitivity and specificity depending on the selected cut point (any increase in sensitivity will be accompanied by a decrease in specificity). The area under an ROC curve (AUC) is a measure for the accuracy of a diagnostic test (the larger the area the better; the optimum is 1; a random test would have a ROC curve lying on the diagonal with an area of 0.5; for reference: J. P. Egan. (1975) Signal Detection Theory and ROC Analysis, Academic Press, New York).

The term “neoplasm” as used herein refers to any new and abnormal growth of tissue. Thus, a neoplasm can be a premalignant neoplasm or a malignant neoplasm.

The term “neoplasm-specific marker,” as used herein, refers to any biological material or element that can be used to indicate the presence of a neoplasm. Examples of biological materials include, without limitation, nucleic acids, polypeptides, carbohydrates, fatty acids, cellular components (e.g., cell membranes and mitochondria), and whole cells. In some instances, markers are particular nucleic acid regions (e.g., genes, intragenic regions, specific loci, etc.). Regions of nucleic acid that are markers may be referred to, e.g., as “marker genes,” “marker regions,” “marker sequences,” “marker loci,” etc.

As used herein, the term “adenoma” refers to a benign tumor of glandular origin. Although these growths are benign, over time they may progress to become malignant.

The term “pre-cancerous” or “pre-neoplastic” and equivalents thereof refer to any cellular proliferative disorder that is undergoing malignant transformation.

A “site” of a neoplasm, adenoma, cancer, etc. is the tissue, organ, cell type, anatomical area, body part, etc. in a subject's body where the neoplasm, adenoma, cancer, etc. is located.

As used herein, a “diagnostic” test application includes the detection or identification of a disease state or condition of a subject, determining the likelihood that a subject will contract a given disease or condition, determining the likelihood that a subject with a disease or condition will respond to therapy, determining the prognosis of a subject with a disease or condition (or its likely progression or regression), and determining the effect of a treatment on a subject with a disease or condition. For example, a diagnostic can be used for detecting the presence or likelihood of a subject contracting a neoplasm or the likelihood that such a subject will respond favorably to a compound (e.g., a pharmaceutical, e.g., a drug) or other treatment.

The term “isolated” when used in relation to a nucleic acid, as in “an isolated oligonucleotide” refers to a nucleic acid sequence that is identified and separated from at least one contaminant nucleic acid with which it is ordinarily associated in its natural source. Isolated nucleic acid is present in a form or setting that is different from that in which it is found in nature. In contrast, non-isolated nucleic acids, such as DNA and RNA, are found in the state they exist in nature. Examples of non-isolated nucleic acids include: a given DNA sequence (e.g., a gene) found on the host cell chromosome in proximity to neighboring genes; RNA sequences, such as a specific mRNA sequence encoding a specific protein, found in the cell as a mixture with numerous other mRNAs which encode a multitude of proteins. However, isolated nucleic acid encoding a particular protein includes, by way of example, such nucleic acid in cells ordinarily expressing the protein, where the nucleic acid is in a chromosomal location different from that of natural cells, or is otherwise flanked by a different nucleic acid sequence than that found in nature. The isolated nucleic acid or oligonucleotide may be present in single-stranded or double-stranded form. When an isolated nucleic acid or oligonucleotide is to be utilized to express a protein, the oligonucleotide will contain at a minimum the sense or coding strand (i.e., the oligonucleotide may be single-stranded), but may contain both the sense and anti-sense strands (i.e., the oligonucleotide may be double-stranded). An isolated nucleic acid may, after isolation from its natural or typical environment, be combined with other nucleic acids or molecules. For example, an isolated nucleic acid may be present in a host cell into which it has been placed, e.g., for heterologous expression.

The term “purified” refers to molecules, either nucleic acid or amino acid sequences that are removed from their natural environment, isolated, or separated. An “isolated nucleic acid sequence” may therefore be a purified nucleic acid sequence. “Substantially purified” molecules are at least 60% free, preferably at least 75% free, and more preferably at least 90% free from other components with which they are naturally associated. As used herein, the terms “purified” or “to purify” also refer to the removal of contaminants from a sample. The removal of contaminating proteins results in an increase in the percent of polypeptide or nucleic acid of interest in the sample. In another example, recombinant polypeptides are expressed in plant, bacterial, yeast, or mammalian host cells and the polypeptides are purified by the removal of host cell proteins; the percent of recombinant polypeptides is thereby increased in the sample.

The term “composition comprising” a given polynucleotide sequence or polypeptide refers broadly to any composition containing the given polynucleotide sequence or polypeptide. The composition may comprise an aqueous solution containing salts (e.g., NaCl), detergents (e.g., SDS), and other components (e.g., Denhardt's solution, dry milk, salmon sperm DNA, etc.).

The term “sample” is used in its broadest sense. In one sense it can refer to an animal cell or tissue. In another sense, it refers to a specimen or culture obtained from any source, as well as biological and environmental samples. Biological samples may be obtained from plants or animals (including humans) and encompass fluids, solids, tissues, and gases. Environmental samples include environmental material such as surface matter, soil, water, and industrial samples. These examples are not to be construed as limiting the sample types applicable to the present invention.

As used herein, a “remote sample” as used in some contexts relates to a sample indirectly collected from a site that is not the cell, tissue, or organ source of the sample. For instance, when sample material originating from the pancreas is assessed in a stool sample the sample is a remote sample.

As used herein, the terms “patient” or “subject” refer to organisms to be subject to various tests provided by the technology. The term “subject” includes animals, preferably mammals, including humans. In a preferred embodiment, the subject is a primate. In an even more preferred embodiment, the subject is a human. Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; pinnipeds; and horses. Thus, also provided is the diagnosis and treatment of livestock, including, but not limited to, domesticated swine, ruminants, ungulates, horses (including race horses), and the like. The presently-disclosed subject matter further includes a system for diagnosing a lung cancer in a subject. The system can be provided, for example, as a commercial kit that can be used to screen for a risk of lung cancer or diagnose a lung cancer in a subject from whom a biological sample has been collected. An exemplary system provided in accordance with the present technology includes assessing the methylation state of a marker described herein.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. As used herein, the term “fragmented kit” refers to delivery systems comprising two or more separate containers that each contain a subportion of the total kit components. The containers may be delivered to the intended recipient together or separately. For example, a first container may contain an enzyme for use in an assay, while a second container contains oligonucleotides. The term “fragmented kit” is intended to encompass kits containing Analyte specific reagents (ASR's) regulated under section 520(e) of the Federal Food, Drug, and Cosmetic Act, but are not limited thereto. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

As used herein, the term “information” refers to any collection of facts or data. In reference to information stored or processed using a computer system(s), including but not limited to internets, the term refers to any data stored in any format (e.g., analog, digital, optical, etc.). As used herein, the term “information related to a subject” refers to facts or data pertaining to a subject (e.g., a human, plant, or animal). The term “genomic information” refers to information pertaining to a genome including, but not limited to, nucleic acid sequences, genes, percentage methylation, allele frequencies, RNA expression levels, protein expression, phenotypes correlating to genotypes, etc. “Allele frequency information” refers to facts or data pertaining to allele frequencies, including, but not limited to, allele identities, statistical correlations between the presence of an allele and a characteristic of a subject (e.g., a human subject), the presence or absence of an allele in an individual or population, the percentage likelihood of an allele being present in an individual having one or more particular characteristics, etc.

### DETAILED DESCRIPTION

Provided herein is technology for screening multiple types of cancer from a biological sample, and particularly, but not exclusively, to methods, compositions, and related uses for simultaneously detecting the presence of multiple types of cancer (e.g., liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer) from a biological sample (e.g., stool sample, tissue sample, organ secretion sample, CSF sample, saliva sample, blood sample, plasma sample or urine sample).

Indeed, as described in Example I, experiments conducted during the course for identifying embodiments for the present invention involved a validation study of the utility and performance of a combined panel of methylated DNA markers (MDMs) and proteins for multicancer detection by testing an independent set of case/control samples with a refined panel of markers. Such experiments resulted in the identification of a set of methylated DNA markers (MDMs), a set of protein markers, and a combination of MDMs and protein markers for simultaneously detecting the presence of multiple types of cancer (e.g., liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer) from a biological sample (e.g., stool sample, tissue sample, organ secretion sample, CSF sample, saliva sample, blood sample, plasma sample or urine sample).

In particular aspects, the present technology provides compositions and methods for identifying, determining, and/or classifying multiple types of cancer from a biological sample (e.g., stool sample, tissue sample, organ secretion sample, CSF sample, saliva sample, blood sample, plasma sample or urine sample). The methods generally comprise determining 1) the methylation status of at least one methylation marker in a biological sample isolated from a subject and/or 2) the expression and/or activity level of at least one protein marker in the biological sample, wherein a change in the methylation state of the marker and/or protein marker expression and/or activity level is indicative of the presence, class, or site of a specific type of cancer. Generally, such methods are not limited to the detection for the presence or absence of specific types of cancer. In some embodiments, the types of cancer include, but are not limited to, liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer.

In certain embodiments of the technology, methods are provided that comprise the following steps:
1) contacting a nucleic acid (e.g., genomic DNA) in a biological sample obtained from a subject with at least one reagent or series of reagents that distinguishes between methylated and non-methylated nucleotides (e.g., CpG dinucleotides) within at least one methylation marker; and/or contacting the biological sample obtained from the subject with a series of reagents necessary to measure the expression and/or activity level of one or more protein markers; and
2) detecting for the presence or absence of multiple types of cancer (e.g., afforded with a sensitivity of greater than or equal to 80% and a specificity of greater than or equal to 80%).

In certain embodiments of the technology, methods are provided that comprise the following steps:
1) measuring one or both of:
   a) a methylation level for one or more genes or methylation markers in a biological sample from a human individual through treating genomic DNA in the biological sample with a reagent that modifies DNA in a methylation-specific manner; and
   b) the expression and/or activity level of one or more protein markers;
2) amplifying the treated genomic DNA using a set of primers for the selected one or more genes or methylation markers; and
3) determining the methylation level of the one or more genes or methylation markers.

In some embodiments of the technology, methods are provided that comprise steps 1-3 and/or step 4:
1) measuring an amount of one or more methylated marker genes in DNA from a biological sample;
2) measuring an amount of at least one reference marker in the DNA;
3) calculating a value for the amount of the at least one methylated marker gene measured in the DNA as a percentage of the amount of the reference marker gene measured in the DNA, wherein the value indicates the amount of the at least one methylated marker DNA measured in the biological sample;
4) measuring the expression and/or activity level of one or more protein markers in the biological sample.

In some embodiments of the technology, methods are provided that comprise steps 1-3 and/or step 4:
1) measuring a methylation level of a CpG site for one or more genes in a biological sample of a human individual through treating genomic DNA in the biological sample with bisulfite a reagent capable of modifying DNA in a methylation-specific manner;
2) amplifying the modified genomic DNA using a set of primers for the selected one or more genes;
3) determining the methylation level of the CpG site for the selected one or more genes;
4) measuring the expression and/or activity level of one or more protein markers in the biological sample.

In certain embodiments, the technology provides methods for characterizing a biological sample comprising:
(a) measuring one or both of:
   i) a methylation level of a CpG site for one or more genes in a biological sample of a human individual through treating genomic DNA in the biological sample with bisulfite; amplifying the bisulfite-treated genomic DNA using a set of primers for the selected one or more genes; and determining the methylation level of the CpG site; and
   ii) an expression and/or activity level of one or more protein markers;
(b) comparing one or both of:
   i) the methylation level to a methylation level of a corresponding set of genes in control samples without a specific type of cancer;
   ii) the expression and/or activity level of the one or more protein markers to an expression and/or activity level of a corresponding set of protein markers in control samples without a specific type of cancer; and
(c) determining that the individual has a specific type of cancer when one or both of:
   i) the methylation level measured in the one or more genes is higher than the methylation level measured in the respective control samples; and
   ii) the expression and/or activity level of one or more protein markers is higher than the expression and/or activity level measured in the respective control samples.

In certain embodiments, the technology provides methods comprising one or both of:
(i) measuring in a biological sample a methylation level of one or more genes through treating genomic DNA in the biological sample with bisulfite; amplifying the bisulfate-treated genomic DNA using a set of primers for the selected one or more genes; and determining the methylation level of the one or more genes; and
(ii) measuring an expression and/or activity level of one or more protein markers.

In certain embodiments, the technology provides methods of screening for one or more types of cancer in a sample obtained from a subject, the method comprising

1) one or both of
i) assaying a methylation state of one or more DNA methylation markers;

and
ii) measuring the expression and/or activity level of one or more protein markers; and
   2) identifying the subject as having one or more types of cancer when:
i) the methylation state of the marker is different than a methylation state of the marker assayed in a subject that does not have the one or more types of cancer; and/or
ii) the expression and/or activity level of one or more protein markers is different than an expression and/or activity level of the protein marker assayed in a subject that does not have the one or more types of cancer.

In certain embodiments, the technology provides methods, comprising:
i) measuring a methylation level for one or more genes in a biological sample of a human individual through treating genomic DNA in the biological sample with a reagent that modifies DNA in a methylation-specific manner; amplifying the treated genomic DNA using a set of primers for the selected one or more genes; and determining the methylation level of the one or more genes; and/or
ii) measuring the expression and/or activity level of one or more protein markers.

In certain embodiments, the technology provides methods for characterizing a biological sample comprising:
a) measuring an amount of at least one methylated marker gene in DNA extracted from the biological sample; treating genomic DNA in the biological sample with bisulfate; amplifying the bisulfate-treated genomic DNA using primers specific for a CpG site for each marker gene, wherein the primers specific for each marker gene are capable of binding an amplicon bound by a primer sequence for the marker gene recited in Table 2, wherein the amplicon bound by the primer sequence for the marker gene recited in Table 2 is at least a portion of a genetic region for the methylated marker gene recited in Table 1; determining the methylation level of the CpG site for one or more genes; and/or
b) measuring in the biological sample an expression and/or activity level of one or more protein markers.

In certain embodiments, the technology provides methods comprising:
a) measuring the methylation level of one or more methylated marker genes in DNA extracted from a biological sample through extracting genomic DNA from a biological sample of a human individual suspected of having or having cancer; treating the extracted genomic DNA with bisulfite, amplifying the bisulfite-treated genomic DNA with primers specific for the one or more genes, wherein the primers specific for the one or more genes are capable of binding at least a portion of the bisulfite-treated genomic DNA for a chromosomal region for the marker recited in Table 1; and measuring the methylation level of one or more methylated marker genes; and/or
b) measuring in the biological sample an expression and/or activity level of one or more protein markers.

In certain embodiments, the technology provides methods comprising:
a) extracting genomic DNA from a biological sample of a human individual suspected of having or having cancer, treating the extracted genomic DNA with bisulfite, amplifying the bisulfite-treated genomic DNA using separate primers specific for CpG sites for one or more of the methylation markers, and measuring a methylation level of the CpG site for each of the one or more methylation markers; and/or
b) measuring in the biological sample an expression and/or activity level of one or more protein markers.

In certain embodiments, the technology provides methods for preparing a DNA fraction from a biological sample of a human individual useful for analyzing one or more genetic loci involved in one or more chromosomal aberrations, comprising:
(a) extracting genomic DNA from a biological sample of a human individual;
(b) producing a fraction of the extracted genomic DNA by:
   (i) treating the extracted genomic DNA with a reagent that modifies DNA in a methylation-specific manner;
   (ii) amplifying the bisulfite-treated genomic DNA using separate primers specific for one or more methylation markers;
(c) analyzing one or more genetic loci in the produced fraction of the extracted genomic DNA by measuring a methylation level of the CpG site for each of the one or more methylation markers.

In certain embodiments, the technology provides methods for preparing a DNA fraction from a biological sample of a human individual useful for analyzing one or more DNA fragments involved in one or more chromosomal aberrations, comprising:

(a) extracting genomic DNA from a biological sample of a human individual;
(b) producing a fraction of the extracted genomic DNA by:
(i) treating the extracted genomic DNA with a reagent that modifies DNA in a methylation-specific manner;
(ii) amplifying the bisulfite-treated genomic DNA using separate primers specific for one or more methylation markers; and
   (c) analyzing one or more DNA fragments in the produced fraction of the extracted genomic DNA by measuring a methylation level of the CpG site for each of the one or more methylation markers.

Such methods are not limited to specific methylated markers, methylated marker genes, genes, DMRs, and/or DNA methylated markers. In some embodiments, the one or more methylated markers, methylated marker genes, genes, DMRs, and/or DNA methylated markers comprise a base in a DMR selected from a group consisting of DMR 1-38 as provided in Table 1.

In some embodiments, the one or more methylated markers, methylated marker genes, genes, DMRs, and/or DNA methylated markers are selected from FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, ST8SIA1, IFFO1, HOXA9, HOPX, OSR2, QKI, RYR2, GPRIN1, ZNF569, CD1D, NTRK3, VAV3, and FAM59B.

In some embodiments, the one or more methylated markers, methylated marker genes, genes, DMRs, and/or DNA methylated markers are selected from FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, and ST8SIA1.

In some embodiments, the one or more methylated markers, methylated marker genes, genes, DMRs, and/or DNA methylated markers are selected from GRIN2D, SHOX2, ZNF671, SIM2, TRH, CAPN2, CHST2_7890, FER1L4, FAIM2, PPP2R5C, TSPYL5, NDRG4, ZNF781, IFFO1, HOXA9, and HOPX.

In some embodiments, the one or more methylated markers, methylated marker genes, genes, DMRs, and/or DNA methylated markers are selected from GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B.

In some embodiments, the one or more methylated markers, methylated marker genes, genes, DMRs, and/or DNA methylated markers are selected from FAIM2, CHST2, ZNF671, GRIN2D, and CDO1.

In some embodiments, the one or more methylated markers, methylated marker genes, genes, DMRs, and/or DNA methylated markers are selected from ZNF671, GRIN2D, NDGR4, SHOX2, and B3GALT6.

In some embodiments, the one or more methylated markers, methylated marker genes, genes, DMRs, and/or DNA methylated markers are selected from CDO1, GRIN2D, SHOX2, OSR2, QKI, SIM2, TRH, CAPN2, SFMBT2, CHST2, ST8SIA1, HOXA1, FER1L4, FAIM2, IFFO1, EMX1, ZNF671, PRKCB, HOXB2, BARX1, PPP2R5C, and TSPYL5.

Such methods are not limited to particular protein markers.

In some embodiments, the one or more protein markers are selected from CEA, CA125, CA19.9, AFP, and CA-15-3.

In some embodiments, the one or more protein markers are selected from CEA, CA125, and CA19.9.

In some embodiments, the one or more protein markers are selected from CEA, CA125, CA19.9, and AFP.

Such methods are not limited to screening for a specific type of cancer.

In some embodiments, the cancer is any type of cancer. A non-limiting exemplary list of cancers pertaining to the described methods include, but is not limited to, pancreatic cancer, acute myeloid leukemia (AML), breast cancer, prostate cancer, lymphoma, skin cancer, colon cancer, melanoma, malignant melanoma, ovarian cancer, brain cancer, primary brain carcinoma, head-neck cancer, glioma, glioblastoma, liver cancer, bladder cancer, non-small cell lung cancer, head or neck carcinoma, breast carcinoma, ovarian carcinoma, lung carcinoma, small-cell lung carcinoma, Wilms' tumor, cervical carcinoma, testicular carcinoma, bladder carcinoma, pancreatic carcinoma, stomach carcinoma, colon carcinoma, prostatic carcinoma, genitourinary carcinoma, thyroid carcinoma, esophageal carcinoma, myeloma, multiple myeloma, adrenal carcinoma, renal cell carcinoma, endometrial carcinoma, adrenal cortex carcinoma, malignant pancreatic insulinoma, malignant carcinoid carcinoma, choriocarcinoma, mycosis fungoides, malignant hypercalcemia, cervical hyperplasia, leukemia, chronic lymphocytic leukemia, acute myelogenous leukemia, chronic myelogenous leukemia, chronic granulocytic leukemia, acute granulocytic leukemia, hairy cell leukemia, neuroblastoma, rhabdomyosarcoma, Kaposi's sarcoma, polycythemia vera, essential thrombocytosis, Hodgkin's disease, non-Hodgkin's lymphoma, soft-tissue sarcoma, osteogenic sarcoma, primary macroglobulinemia, and retinoblastoma. In some embodiments, the types of cancer include, but are not limited to, liver cancer, esophageal cancer, lung cancer, ovarian cancer, pancreatic cancer, and gastric cancer.

In some embodiments, the cancer is selected from liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer.

In some embodiments, the cancer is selected from liver cancer, esophageal cancer, lung cancer, ovarian cancer, pancreatic cancer, gastric cancer, bladder cancer, breast cancer, cervical cancer, colorectal cancer, renal cancer, and uterine cancer.

Such methods are not limited to a specific sample or biological sample type. For example, in some embodiments the sample or biological sample is a stool sample, a tissue sample, a blood sample (e.g., stool sample, tissue sample, organ secretion sample, CSF sample, saliva sample, blood sample, plasma sample or urine sample), an excretion, or a urine sample. In some embodiments, the sample comprises blood, serum, plasma, gastric secretions, pancreatic juice, a cerebral spinal fluid (CSF) sample, a gastrointestinal biopsy sample, and/or cells recovered from stool. The sample or biological sample may include cells, secretions, or tissues from the lymph gland, breast, liver, bile ducts, pancreas, stomach, colon, rectum, esophagus, small intestine, appendix, duodenum, polyps, gall bladder, anus, and/or peritoneum. In some embodiments, the sample or biological sample comprises cellular fluid, ascites, urine, feces, gastric section, pancreatic fluid, fluid obtained during endoscopy, blood, mucus, or saliva.

Various cancers are predicted by various combinations of markers, e.g., as identified by statistical techniques related to specificity and sensitivity of prediction. The technology further provides methods for identifying predictive combinations and validated predictive combinations for some cancers.

Such methods are not limited to a subject type. In some embodiments, the subject is a mammal. In some embodiments, the subject is a human.

Such methods are not limited to a particular manner or technique for measuring protein expression and/or activity. Techniques for measuring protein expression and/or activity levels are known in the art. Indeed, any known technique for measuring protein expression and/or activity levels are contemplated and herein incorporated.

Such methods are not limited to a particular manner or technique for determining characterizing, measuring, or assaying methylation for one or more methylated markers, methylated marker genes, genes, DMRs, and/or DNA methylated markers. In some embodiments, such techniques are based upon an analysis of the methylation status (e.g., CpG methylation status) of at least one marker, region of a marker, or base of a marker comprising a DMR.

In some embodiments, measuring the methylation state of a methylation marker in a sample comprises determining the methylation state of one base. In some embodiments, measuring the methylation state of the marker in the sample comprises determining the extent of methylation at a plurality of bases. Moreover, in some embodiments, the methylation state of the methylated marker comprises an increase in methylation of the marker relative to a normal methylation state of the marker. In some embodiments, the methylation state of the marker comprises a decreased methylation of the marker relative to a normal methylation state of the marker. In some embodiments the methylation state of the marker comprises a different pattern of methylation of the marker relative to a normal methylation state of the marker.

Furthermore, in some embodiments the marker is a region of 100 or fewer bases, the marker is a region of 500 or fewer bases, the marker is a region of 1000 or fewer bases, the marker is a region of 5000 or fewer bases, or, in some embodiments, the marker is one base. In some embodiments the marker is in a high CpG density promoter.

In certain embodiments, methods for analyzing a nucleic acid for the presence of 5-methylcytosine involves treatment of DNA with a reagent that modifies DNA in a methylation-specific manner. Examples of such reagents include, but are not limited to, a methylation-sensitive restriction enzyme, a methylation-dependent restriction enzyme, a bisulfite reagent, a TET enzyme, and a borane reducing agent.

A frequently used method for analyzing a nucleic acid for the presence of 5-methylcytosine is based upon the bisulfite method described by Frommer, et al. for the detection of 5-methylcytosines in DNA (Frommer et al. (1992) Proc. Natl. Acad. Sci. USA 89: 1827-31 explicitly incorporated herein by reference in its entirety for all purposes) or variations thereof. The bisulfite method of mapping 5-methylcytosines is based on the observation that cytosine, but not 5-methylcytosine, reacts with hydrogen sulfite ion (also known as bisulfite). The reaction is usually performed according to the following steps: first, cytosine reacts with hydrogen sulfite to form a sulfonated cytosine. Next, spontaneous deamination of the sulfonated reaction intermediate results in a sulfonated uracil. Finally, the sulfonated uracil is desulfonated under alkaline conditions to form uracil. Detection is possible because uracil base pairs with adenine (thus behaving like thymine), whereas 5-methylcytosine base pairs with guanine (thus behaving like cytosine). This makes the discrimination of methylated cytosines from non-methylated cytosines possible by, e.g., bisulfite genomic sequencing (Grigg G, & Clark S, Bioessays (1994) 16: 431-36; Grigg G, DNA Seq. (1996) 6: 189-98), methylation-specific PCR (MSP) as is disclosed, e.g., in U.S. Pat. No. 5,786,146, or using an assay comprising sequence-specific probe cleavage, e.g., a QuARTS flap endonuclease assay (see, e.g., Zou et al. (2010) “Sensitive quantification of methylated markers with a novel methylation specific technology” Clin Chem 56: A199; and in U.S. Pat. Nos. 8,361,720; 8,715,937; 8,916,344; and 9,212,392.

Some conventional technologies are related to methods comprising enclosing the DNA to be analyzed in an agarose matrix, thereby preventing the diffusion and renaturation of the DNA (bisulfite only reacts with single-stranded DNA), and replacing precipitation and purification steps with a fast dialysis (Olek A, et al. (1996) “A modified and improved method for bisulfite based cytosine methylation analysis” Nucleic Acids Res. 24: 5064-6). It is thus possible to analyze individual cells for methylation status, illustrating the utility and sensitivity of the method. An overview of conventional methods for detecting 5-methylcytosine is provided by Rein, T., et al. (1998) Nucleic Acids Res. 26: 2255.

The bisulfite technique typically involves amplifying short, specific fragments of a known nucleic acid subsequent to a bisulfite treatment, then either assaying the product by sequencing (Olek & Walter (1997) Nat. Genet. 17: 275-6) or a primer extension reaction (Gonzalgo & Jones (1997) Nucleic Acids Res. 25: 2529-31; WO 95/00669; U.S. Pat. No. 6,251,594) to analyze individual cytosine positions. Some methods use enzymatic digestion (Xiong & Laird (1997) Nucleic Acids Res. 25: 2532-4). Detection by hybridization has also been described in the art (Olek et al., WO 99/28498). Additionally, use of the bisulfite technique for methylation detection with respect to individual genes has been described (Grigg & Clark (1994) Bioessays 16: 431-6; Zeschnigk et al. (1997) Hum Mol Genet. 6: 387-95; Feil et al. (1994) Nucleic Acids Res. 22: 695; Martin et al. (1995) Gene 157: 261-4; WO 9746705; WO 9515373).

Various methylation assay procedures can be used in conjunction with bisulfite treatment according to the present technology. These assays allow for determination of the methylation state of one or a plurality of CpG dinucleotides (e.g., CpG islands) within a nucleic acid sequence. Such assays involve, among other techniques, sequencing of bisulfite-treated nucleic acid, PCR (for sequence-specific amplification), Southern blot analysis, and use of methylation-specific restriction enzymes, e.g., methylation-sensitive or methylation-dependent enzymes.

For example, genomic sequencing has been simplified for analysis of methylation patterns and 5-methylcytosine distributions by using bisulfite treatment (Frommer et al. (1992) Proc. Natl. Acad. Sci. USA 89: 1827-1831). Additionally, restriction enzyme digestion of PCR products amplified from bisulfite-converted DNA finds use in assessing methylation state, e.g., as described by Sadri & Hornsby (1997) Nucl. Acids Res. 24: 5058-5059 or as embodied in the method known as COBRA (Combined Bisulfite Restriction Analysis) (Xiong & Laird (1997) Nucleic Acids Res. 25: 2532-2534).

COBRA™ analysis is a quantitative methylation assay useful for determining DNA methylation levels at specific loci in small amounts of genomic DNA (Xiong & Laird, Nucleic Acids Res. 25:2532-2534, 1997). Briefly, restriction enzyme digestion is used to reveal methylation-dependent sequence differences in PCR products of sodium bisulfite-treated DNA. Methylation-dependent sequence differences are first introduced into the genomic DNA by standard bisulfite treatment according to the procedure described by Frommer et al. (Proc. Natl. Acad. Sci. USA 89:1827-1831, 1992). PCR amplification of the bisulfite converted DNA is then performed using primers specific for the CpG islands of interest, followed by restriction endonuclease digestion, gel electrophoresis, and detection using specific, labeled hybridization probes. Methylation levels in the original DNA sample are represented by the relative amounts of digested and undigested PCR product in a linearly quantitative fashion across a wide spectrum of DNA methylation levels. In addition, this technique can be reliably applied to DNA obtained from microdissected paraffin-embedded tissue samples.

Typical reagents (e.g., as might be found in a typical COBRA™-based kit) for COBRA™ analysis may include, but are not limited to: PCR primers for specific loci (e.g., specific genes, markers, DMR, regions of genes, regions of markers, bisulfite treated DNA sequence, CpG island, etc.); restriction enzyme and appropriate buffer; gene-hybridization oligonucleotide; control hybridization oligonucleotide; kinase labeling kit for oligonucleotide probe; and labeled nucleotides. Additionally, bisulfite conversion reagents may include: DNA denaturation buffer; sulfonation buffer; DNA recovery reagents or kits (e.g., precipitation, ultrafiltration, affinity column); desulfonation buffer; and DNA recovery components. Assays such as “MethyLight™” (a fluorescence-based real-time PCR technique) (Eads et al., Cancer Res. 59:2302-2306, 1999), Ms-SNuPE™ (Methylation-sensitive Single Nucleotide Primer Extension) reactions (Gonzalgo & Jones, Nucleic Acids Res. 25:2529-2531, 1997), methylation-specific PCR (“MSP”; Herman et al., Proc. Natl. Acad. Sci. USA 93:9821-9826, 1996; U.S. Pat. No. 5,786,146), and methylated CpG island amplification (“MCA”; Toyota et al., Cancer Res. 59:2307-12, 1999) are used alone or in combination with one or more of these methods.

The “HeavyMethyl™” assay, technique is a quantitative method for assessing methylation differences based on methylation-specific amplification of bisulfite-treated DNA. Methylation-specific blocking probes (“blockers”) covering CpG positions between, or covered by, the amplification primers enable methylation-specific selective amplification of a nucleic acid sample.

The term “HeavyMethyl™ MethyLight™” assay refers to a HeavyMethyl™ MethyLight™ assay, which is a variation of the MethyLight™ assay, wherein the MethyLight™ assay is combined with methylation specific blocking probes covering CpG positions between the amplification primers. The HeavyMethyl™ assay may also be used in combination with methylation specific amplification primers.

Typical reagents (e.g., as might be found in a typical MethyLight™-based kit) for HeavyMethyl™ analysis may include, but are not limited to: PCR primers for specific loci (e.g., specific genes, markers, regions of genes, regions of markers, bisulfite treated DNA sequence, CpG island, or bisulfite treated DNA sequence or CpG island, etc.); blocking oligonucleotides; optimized PCR buffers and deoxynucleotides; and Taq polymerase. MSP (methylation-specific PCR) allows for assessing the methylation status of virtually any group of CpG sites within a CpG island, independent of the use of methylation-sensitive restriction enzymes (Herman et al. Proc. Natl. Acad. Sci. USA 93:9821-9826, 1996; U.S. Pat. No. 5,786,146). Briefly, DNA is modified by sodium bisulfite, which converts unmethylated, but not methylated cytosines, to uracil, and the products are subsequently amplified with primers specific for methylated versus unmethylated DNA. MSP requires only small quantities of DNA, is sensitive to 0.1% methylated alleles of a given CpG island locus, and can be performed on DNA extracted from paraffin-embedded samples. Typical reagents (e.g., as might be found in a typical MSP-based kit) for MSP analysis may include, but are not limited to: methylated and unmethylated PCR primers for specific loci (e.g., specific genes, markers, regions of genes, regions of markers, bisulfite treated DNA sequence, CpG island, etc.); optimized PCR buffers and deoxynucleotides, and specific probes.

The MethyLight™ assay is a high-throughput quantitative methylation assay that utilizes fluorescence-based real-time PCR (e.g., TaqMan®) that requires no further manipulations after the PCR step (Eads et al., Cancer Res. 59:2302-2306, 1999). Briefly, the MethyLight™ process begins with a mixed sample of genomic DNA that is converted, in a sodium bisulfite reaction, to a mixed pool of methylation-dependent sequence differences according to standard procedures (the bisulfite process converts unmethylated cytosine residues to uracil). Fluorescence-based PCR is then performed in a “biased” reaction, e.g., with PCR primers that overlap known CpG dinucleotides. Sequence discrimination occurs both at the level of the amplification process and at the level of the fluorescence detection process.

The MethyLight™ assay is used as a quantitative test for methylation patterns in a nucleic acid, e.g., a genomic DNA sample, wherein sequence discrimination occurs at the level of probe hybridization. In a quantitative version, the PCR reaction provides for a methylation specific amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (e.g., a fluorescence-based version of the HeavyMethyl™ and MSP techniques) or with oligonucleotides covering potential methylation sites.

The MethyLight™ process is used with any suitable probe (e.g. a “TaqMan®” probe, a Lightcycler® probe, etc.) For example, in some applications double-stranded genomic DNA is treated with sodium bisulfite and subjected to one of two sets of PCR reactions using TaqMan® probes, e.g., with MSP primers and/or HeavyMethyl blocker oligonucleotides and a TaqMan® probe. The TaqMan® probe is dual-labeled with fluorescent “reporter” and “quencher” molecules and is designed to be specific for a relatively high GC content region so that it melts at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system.

Typical reagents (e.g., as might be found in a typical MethyLight™-based kit) for MethyLight™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The QM™ (quantitative methylation) assay is an alternative quantitative test for methylation patterns in genomic DNA samples, wherein sequence discrimination occurs at the level of probe hybridization. In this quantitative version, the PCR reaction provides for unbiased amplification in the presence of a fluorescent probe that overlaps a particular putative methylation site. An unbiased control for the amount of input DNA is provided by a reaction in which neither the primers, nor the probe, overlie any CpG dinucleotides. Alternatively, a qualitative test for genomic methylation is achieved by probing the biased PCR pool with either control oligonucleotides that do not cover known methylation sites (a fluorescence-based version of the HeavyMethyl™ and MSP techniques) or with oligonucleotides covering potential methylation sites.

The QM™ process can be used with any suitable probe, e.g., “TaqMan®” probes, Lightcycler® probes, in the amplification process. For example, double-stranded genomic DNA is treated with sodium bisulfite and subjected to unbiased primers and the TaqMan® probe. The TaqMan® probe is dual-labeled with fluorescent “reporter” and “quencher” molecules, and is designed to be specific for a relatively high GC content region so that it melts out at about a 10° C. higher temperature in the PCR cycle than the forward or reverse primers. This allows the TaqMan® probe to remain fully hybridized during the PCR annealing/extension step. As the Taq polymerase enzymatically synthesizes a new strand during PCR, it will eventually reach the annealed TaqMan® probe. The Taq polymerase 5′ to 3′ endonuclease activity will then displace the TaqMan® probe by digesting it to release the fluorescent reporter molecule for quantitative detection of its now unquenched signal using a real-time fluorescent detection system. Typical reagents (e.g., as might be found in a typical QM™-based kit) for QM™ analysis may include, but are not limited to: PCR primers for specific loci (e.g., specific genes, markers, regions of genes, regions of markers, bisulfite treated DNA sequence, CpG island, etc.); TaqMan® or Lightcycler® probes; optimized PCR buffers and deoxynucleotides; and Taq polymerase.

The Ms-SNuPE™ technique is a quantitative method for assessing methylation differences at specific CpG sites based on bisulfite treatment of DNA, followed by single-nucleotide primer extension (Gonzalgo & Jones, Nucleic Acids Res. 25:2529-2531, 1997). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site of interest. Small amounts of DNA can be analyzed (e.g., microdissected pathology sections) and it avoids utilization of restriction enzymes for determining the methylation status at CpG sites.

Typical reagents (e.g., as might be found in a typical Ms-SNuPE™-based kit) for Ms-SNuPE™ analysis may include, but are not limited to: PCR primers for specific loci (e.g., specific genes, markers, regions of genes, regions of markers, bisulfite treated DNA sequence, CpG island, etc.); optimized PCR buffers and deoxynucleotides; gel extraction kit; positive control primers; Ms-SNuPE™ primers for specific loci; reaction buffer (for the Ms-SNuPE reaction); and labeled nucleotides. Additionally, bisulfite conversion reagents may include: DNA denaturation buffer; sulfonation buffer; DNA recovery reagents or kit (e.g., precipitation, ultrafiltration, affinity column); desulfonation buffer; and DNA recovery components.

Reduced Representation Bisulfite Sequencing (RRBS) begins with bisulfite treatment of nucleic acid to convert all unmethylated cytosines to uracil, followed by restriction enzyme digestion (e.g., by an enzyme that recognizes a site including a CG sequence such as Mspl) and complete sequencing of fragments after coupling to an adapter ligand. The choice of restriction enzyme enriches the fragments for CpG dense regions, reducing the number of redundant sequences that may map to multiple gene positions during analysis. As such, RRBS reduces the complexity of the nucleic acid sample by selecting a subset (e.g., by size selection using preparative gel electrophoresis) of restriction fragments for sequencing. As opposed to whole-genome bisulfite sequencing, every fragment produced by the restriction enzyme digestion contains DNA methylation information for at least one CpG dinucleotide. As such, RRBS enriches the sample for promoters, CpG islands, and other genomic features with a high frequency of restriction enzyme cut sites in these regions and thus provides an assay to assess the methylation state of one or more genomic loci.

A typical protocol for RRBS comprises the steps of digesting a nucleic acid sample with a restriction enzyme such as Mspl, filling in overhangs and A-tailing, ligating adaptors, bisulfite conversion, and PCR. See, e.g., et al. (2005) “Genome-scale DNA methylation mapping of clinical samples at single-nucleotide resolution” Nat Methods 7: 133-6; Meissner et al. (2005) “Reduced representation bisulfite sequencing for comparative high-resolution DNA methylation analysis” Nucleic Acids Res. 33: 5868-77.

In some embodiments, a quantitative allele-specific real-time target and signal amplification (QuARTS) assay is used to evaluate methylation state. Three reactions sequentially occur in each QuARTS assay, including amplification (reaction 1) and target probe cleavage (reaction 2) in the primary reaction; and FRET cleavage and fluorescent signal generation (reaction 3) in the secondary reaction. When target nucleic acid is amplified with specific primers, a specific detection probe with a flap sequence loosely binds to the amplicon. The presence of the specific invasive oligonucleotide at the target binding site causes a 5′ nuclease, e.g., a FEN-1 endonuclease, to release the flap sequence by cutting between the detection probe and the flap sequence. The flap sequence is complementary to a non-hairpin portion of a corresponding FRET cassette. Accordingly, the flap sequence functions as an invasive oligonucleotide on the FRET cassette and effects a cleavage between the FRET cassette fluorophore and a quencher, which produces a fluorescent signal. The cleavage reaction can cut multiple probes per target and thus release multiple fluorophores per flap, providing exponential signal amplification. QuARTS can detect multiple targets in a single reaction well by using FRET cassettes with different dyes. See, e.g., in Zou et al. (2010) “Sensitive quantification of methylated markers with a novel methylation specific technology” Clin Chem 56: A199), and U.S. Pat. Nos. 8,361,720; 8,715,937; 8,916,344; and 9,212,392, each of which is incorporated herein by reference for all purposes.

The term “bisulfite reagent” refers to a reagent comprising bisulfite, disulfite, hydrogen sulfite, or combinations thereof, useful as disclosed herein to distinguish between methylated and unmethylated CpG dinucleotide sequences. Methods of said treatment are known in the art (e.g., PCT/EP2004/011715 and WO 2013/116375, each of which is incorporated by reference in its entirety). In some embodiments, bisulfite treatment is conducted in the presence of denaturing solvents such as but not limited to n-alkyleneglycol or diethylene glycol dimethyl ether (DME), or in the presence of dioxane or dioxane derivatives. In some embodiments the denaturing solvents are used in concentrations between 1% and 35% (v/v). In some embodiments, the bisulfite reaction is carried out in the presence of scavengers such as but not limited to chromane derivatives, e.g., 6-hydroxy-2,5,7,8,-tetramethylchromane 2-carboxylic acid or trihydroxybenzone acid and derivates thereof, e.g., Gallic acid (see: PCT/EP2004/011715, which is incorporated by reference in its entirety). In certain preferred embodiments, the bisulfite reaction comprises treatment with ammonium hydrogen sulfite, e.g., as described in WO 2013/116375.

In some embodiments, fragments of the treated DNA are amplified using sets of primer oligonucleotides according to the present invention (e.g., see Table 2) and an amplification enzyme. The amplification of several DNA segments can be carried out simultaneously in one and the same reaction vessel. Typically, the amplification is carried out using a polymerase chain reaction (PCR). Amplicons are typically 100 to 2000 base pairs in length.

In another embodiment of the method, the methylation status of CpG positions within or near a marker comprising a DMR (e.g., DMR 1-38, Table 1) may be detected by use of methylation-specific primer oligonucleotides. This technique (MSP) has been described in U.S. Pat. No. 6,265,171 to Herman. The use of methylation status specific primers for the amplification of bisulfite treated DNA allows the differentiation between methylated and unmethylated nucleic acids. MSP primer pairs contain at least one primer that hybridizes to a bisulfite treated CpG dinucleotide. Therefore, the sequence of said primers comprises at least one CpG dinucleotide. MSP primers specific for non-methylated DNA contain a “T” at the position of the C position in the CpG.

Such methods are not limited to a specific type or kind of primer or primer pair related to the one or more methylated markers, methylated marker genes, genes, DMRs, and/or DNA methylated markers. In some embodiments, the primer or primer pair is recited in Table 2 (SEQ ID Nos: 1-126). In some embodiments, the primer or primer pair specific for each methylated marker gene are capable of binding an amplicon bound by a primer sequence for the marker gene recited in Table 2, wherein the amplicon bound by the primer sequence for the marker gene recited in Table 2 is at least a portion of a genetic region for the methylated marker gene recited in Table 1. In some embodiments, the primer or primer pair for a methylated marker is a set of primers that specifically binds at least a portion of a genetic region comprising chromosomal coordinates for the specific methylated marker recited in Table 1.

In another embodiment, the invention provides a method for converting an oxidized 5-methylcytosine residue in cell-free DNA to a dihydrouracil residue (see, Liu et al., 2019, Nat Biotechnol. 37, pp. 424-429; U.S. Patent Application Publication No. 202000370114). The method involves reaction of an oxidized 5mC residue selected from 5-formylcytosine (5fC), 5-carboxymethylcytosine (5caC), and combinations thereof, with a borane reducing agent. The oxidized 5mC residue may be naturally occurring or, more typically, the result of a prior oxidation of a 5mC or 5hmC residue, e.g., oxidation of 5mC or 5hmC with a TET family enzyme (e.g., TET1, TET2, or TET3), or chemical oxidation of 5 mC or 5hmC, e.g., with potassium perruthenate (KRuO4) or an inorganic peroxo compound or composition such as peroxotungstate (see, e.g., Okamoto et al. (2011) Chem. Commun. 47:11231-33) and a copper (II) perchlorate/2,2,6,6-tetramethylpiperidine-1-oxyl (TEMPO) combination (see Matsushita et al. (2017) Chem. Commun. 53:5756-59).

The borane reducing agent may be characterized as a complex of borane and a nitrogen-containing compound selected from nitrogen heterocycles and tertiary amines. The nitrogen heterocycle may be monocyclic, bicyclic, or polycyclic, but is typically monocyclic, in the form of a 5- or 6-membered ring that contains a nitrogen heteroatom and optionally one or more additional heteroatoms selected from N, O, and S. The nitrogen heterocycle may be aromatic or alicyclic. Preferred nitrogen heterocycles herein include 2-pyrroline, 2H-pyrrole, 1H-pyrrole, pyrazolidine, imidazolidine, 2-pyrazoline, 2-imidazoline, pyrazole, imidazole, 1,2,4-triazole, 1,2,4-triazole, pyridazine, pyrimidine, pyrazine, 1,2,4-triazine, and 1,3,5-triazine, any of which may be unsubstituted or substituted with one or more non-hydrogen substituents. Typical non-hydrogen substituents are alkyl groups, particularly lower alkyl groups, such as methyl, ethyl, n-propyl, isopropyl, n-butyl, isobutyl, t-butyl, and the like. Exemplary compounds include pyridine borane, 2-methylpyridine borane (also referred to as 2-picoline borane), and 5-ethyl-2-pyridine.

The reaction of the borane reducing agent with the oxidized 5mC residue in cell-free DNA is advantageous insofar as non-toxic reagents and mild reaction conditions can be employed; there is no need for any bisulfate, nor for any other potentially DNA-degrading reagents. Furthermore, conversion of an oxidized 5mC residue to dihydrouracil with the borane reducing agent can be carried out without need for isolation of any intermediates, in a “one-pot” or “one-tube” reaction. This is quite significant, since the conversion involves multiple steps, i.e., (1) reduction of the alkene bond linking C-4 and C-5 in the oxidized 5mC, (2) deamination, and (3) either decarboxylation, if the oxidized 5mC is 5caC, or deformylation, if the oxidized 5mC is 5fC.

In addition to a method for converting an oxidized 5-methylcytosine residue in cell-free DNA to a dihydrouracil residue, the invention also provides a reaction mixture related to the aforementioned method. The reaction mixture comprises a sample of cell-free DNA containing at least one oxidized 5-methylcytosine residue selected from 5caC, 5fC, and combinations thereof, and a borane reducing agent effective to effective to reduce, deaminate, and either decarboxylate or deformylate the at least one oxidized 5-methylcytosine residue. The borane reducing agent is a complex of borane and a nitrogen-containing compound selected from nitrogen heterocycles and tertiary amines, as explained above. In a preferred embodiment, the reaction mixture is substantially free of bisulfite, meaning substantially free of bisulfite ion and bisulfite salts. Ideally, the reaction mixture contains no bisulfite.

In a related aspect of the invention, a kit is provided for converting 5mC residues in cell-free DNA to dihydrouracil residues, where the kit includes a reagent for blocking 5hmC residues, a reagent for oxidizing 5mC residues beyond hydroxymethylation to provide oxidized 5mC residues, and a borane reducing agent effective to reduce, deaminate, and either decarboxylate or deformylate the oxidized 5mC residues. The kit may also include instructions for using the components to carry out the above-described method.

In another embodiment, a method is provided that makes use of the above-described oxidation reaction. The method enables detecting the presence and location of 5-methylcytosine residues in cell-free DNA, and comprises the following steps:
(a) modifying 5hmC residues in fragmented, adapter-ligated cell-free DNA to provide an affinity tag thereon, wherein the affinity tag enables removal of modified 5hmC-containing DNA from the cell-free DNA;
(b) removing the modified 5hmC-containing DNA from the cell-free DNA, leaving DNA containing unmodified 5mC residues;
(c) oxidizing the unmodified 5mC residues to give DNA containing oxidized 5mC residues selected from 5caC, 5fC, and combinations thereof;
(d) contacting the DNA containing oxidized 5mC residues with a borane reducing agent effective to reduce, deaminate, and either decarboxylate or deformylate the oxidized 5mC residues, thereby providing DNA containing dihydrouracil residues in place of the oxidized 5mC residues;
(e) amplifying and sequencing the DNA containing dihydrouracil residues;
(0 determining a 5-methylation pattern from the sequencing results in (e).

In another embodiments, a method is provided for identifying 5-methylcytosine (5mC) or 5-hydroxymethylcytosine (5hmC) in a target nucleic acid comprising the steps of:
providing a biological sample comprising the target nucleic acid;
modifying the target nucleic acid comprising the steps of:
   converting the 5mC and 5hmC in the nucleic acid sample to 5-carboxylcytosine (5caC) and/or 5-formylcytosine (5fC) by contacting the nucleic acid sample with a TET enzyme so that one or more 5caC or 5fC residues are generated; and
   converting the 5caC and/or 5fC to dihydrouracil (DHU) by treating the target nucleic acid with a borane reducing agent to provide a modified nucleic acid sample comprising a modified target nucleic acid; and
detecting the sequence of the modified target nucleic acid; wherein a cytosine (C) to thymine (T) transition or a cytosine (C) to DHU transition in the sequence of the modified target nucleic acid compared to the target nucleic acid provides the location of either a 5mC or 5hmC in the target nucleic acid.

In some embodiments, the borane reducing agent is 2-picoline borane.

In some embodiments, the step of detecting the sequence of the modified target nucleic acid comprises one or more of chain termination sequencing, microarray, high-throughput sequencing, and restriction enzyme analysis.

In some embodiments, the TET enzyme is selected from the group consisting of human TET1, TET2, and TET3; murine Tet1, Tet2, and Tet3; Naegleria TET (NgTET);

and Coprinopsis cinerea (CcTET).

In some embodiments, the method further comprises a step of blocking one or more modified cytosines. In some embodiments, the step of blocking comprises adding a sugar to a 5hmC.

In some embodiments, the method further comprises a step of amplifying the copy number of one or more nucleic acid sequences.

In some embodiments, the oxidizing agent is potassium perruthenate or Cu(II)/TEMPO (2,2,6,6-tetramethylpiperidine-1-oxyl.)

The cell-free DNA is extracted from a body sample from a subject, where the body sample is typically whole blood, plasma, or serum, most typically plasma, but the sample may also be urine, saliva, mucosal excretions, sputum, stool, or tears. In some embodiments, the cell-free DNA is derived from a tumor. In other embodiments, the cell-free DNA is from a patient with a disease or other pathogenic condition. The cell-free DNA may or may not derive from a tumor. In step (a), it should be noted that the cell-free DNA in which 5hmC residues are to be modified is in purified, fragmented form, and adapter-ligated. DNA purification in this context can be carried out using any suitable method known to those of ordinary skill in the art and/or described in the pertinent literature, and, while cell-free DNA can itself be highly fragmented, further fragmentation may occasionally be desirable, as described, for example, in U.S. Patent Publication No. 2017/0253924. The cell-free DNA fragments are generally in the size range of about 20 nucleotides to about 500 nucleotides, more typically in the range of about 20 nucleotides to about 250 nucleotides. The purified cell-free DNA fragments that are modified in step (a) have been end-repaired using conventional means (e.g., a restriction enzyme) so that the fragments have a blunt end at each 3′ and 5′ terminus. In a preferred method, as described in WO 2017/176630, the blunted fragments have also been provided with a 3′ overhang comprising a single adenine residue using a polymerase such as Taq polymerase. This facilitates subsequent ligation of a selected universal adapter, i.e., an adapter such as a Y-adapter or a hairpin adapter that ligates to both ends of the cell-free DNA fragments and contains at least one molecular barcode. Use of adapters also enables selective PCR enrichment of adapter-ligated DNA fragments.

In step (a), then, the “purified, fragmented cell-free DNA” comprises adapter-ligated DNA fragments. Modification of 5hmC residues in these cell-free DNA fragments with an affinity tag, as specified in step (a), is done so as to enable subsequent removal of the modified 5hmC-containing DNA from the cell-free DNA. In one embodiment, the affinity tag comprises a biotin moiety, such as biotin, desthiobiotin, oxybiotin, 2-iminobiotin, diaminobiotin, biotin sulfoxide, biocytin, or the like. Use of a biotin moiety as the affinity tag allows for facile removal with streptavidin, e.g., streptavidin beads, magnetic streptavidin beads, etc.

Tagging 5hmC residues with a biotin moiety or other affinity tag is accomplished by covalent attachment of a chemoselective group to 5hmC residues in the DNA fragments, where the chemoselective group is capable of undergoing reaction with a functionalized affinity tag so as to link the affinity tag to the 5hmC residues. In one embodiment, the chemoselective group is UDP glucose-6-azide, which undergoes a spontaneous 1,3-cycloaddition reaction with an alkyne-functionalized biotin moiety, as described in Robertson et al. (2011) Biochem. Biophys. Res. Comm. 411(1):40-3, U.S. Pat. No. 8,741,567, and WO 2017/176630. Addition of an alkyne-functionalized biotin-moiety thus results in covalent attachment of the biotin moiety to each 5hmC residue.

The affinity-tagged DNA fragments can then be pulled down in step (b) using, in one embodiment, streptavidin, in the form of streptavidin beads, magnetic streptavidin beads, or the like, and set aside for later analysis, if so desired. The supernatant remaining after removal of the affinity-tagged fragments contains DNA with unmodified 5mC residues and no 5hmC residues.

In step (c), the unmodified 5mC residues are oxidized to provide 5caC residues and/or 5fC residues, using any suitable means. The oxidizing agent is selected to oxidize 5mC residues beyond hydroxymethylation, i.e., to provide 5caC and/or 5fC residues. Oxidation may be carried out enzymatically, using a catalytically active TET family enzyme. A “TET family enzyme” or a “TET enzyme” as those terms are used herein refer to a catalytically active “TET family protein” or a “TET catalytically active fragment” as defined in U.S. Pat. No. 9,115,386, the disclosure of which is incorporated by reference herein. A preferred TET enzyme in this context is TET2; see Ito et al. (2011) Science 333(6047):1300-1303. Oxidation may also be carried out chemically, as described in the preceding section, using a chemical oxidizing agent. Examples of suitable oxidizing agent include, without limitation: a perruthenate anion in the form of an inorganic or organic perruthenate salt, including metal perruthenates such as potassium perruthenate (KRuO4), tetraalkylammonium perruthenates such as tetrapropylammonium perruthenate (TPAP) and tetrabutylammonium perruthenate (TBAP), and polymer supported perruthenate (PSP); and inorganic peroxo compounds and compositions such as peroxotungstate or a copper (II) perchlorate/TEMPO combination. It is unnecessary at this point to separate 5fC-containing fragments from 5caC-containing fragments, insofar as in the next step of the process, step (e) converts both 5fC residues and 5caC residues to dihydrouracil (DHU).

In some embodiments, 5-hydroxymethylcytosine residues are blocked with β-glucosyltransferase (β3GT), while 5-methylcytosine residues are oxidized with a TET enzyme effective to provide a mixture of 5-formylcytosine and 5-carboxymethylcytosine. The mixture containing both of these oxidized species can be reacted with 2-picoline borane or another borane reducing agent to give dihydrouracil. In a variation on this embodiment, 5hmC-containing fragments are not removed in step (b). Rather, “TET-Assisted Picoline Borane Sequencing (TAPS),” 5mC-containing fragments and 5hmC-containing fragments are together enzymatically oxidized to provide 5fC- and 5caC-containing fragments. Reaction with 2-picoline borane results in DHU residues wherever 5mC and 5hmC residues were originally present. “Chemical Assisted Picoline Borane Sequencing (CAPS),” involves selective oxidation of 5hmC-containing fragments with potassium perruthenate, leaving 5mC residues unchanged.

There are numerous advantages to the method of this embodiment: bisulfite is unnecessary, nontoxic reagents and reactants are employed; and the process proceeds under mild conditions. In addition, the entire process can be performed in a single tube, without need for isolation of any intermediates.

In a related embodiment, the above method includes a further step: (g) identifying a hydroxymethylation pattern in the 5hmC-containing DNA removed from the cell-free DNA in step (b). This can be carried out using the techniques described in detail in WO 2017/176630. The process can be carried out without removal or isolation of intermediates in a one-tube method. For example, initially, cell-free DNA fragments, preferably adapter-ligated DNA fragments, are subjected to functionalization with βGT-catalyzed uridine diphosphoglucose 6-azide, followed by biotinylation via the chemoselective azide groups. This procedure results in covalently attached biotin at each 5hmC site. In a next step, the biotinylated strands and strands containing unmodified (native) 5mC are pulled down simultaneously for further processing. The native 5mC-containing strands are pulled down using an anti-5mC antibody or a methyl-CpG-binding domain (MBD) protein, as is known in the art. Then, with the 5hmC residues blocked, the unmodified 5mC residues are selectively oxidized using any suitable technique for converting 5mC to 5fC and/or 5caC, as described elsewhere herein.

The fragments obtained by means of the amplification can carry a directly or indirectly detectable label. In some embodiments, the labels are fluorescent labels, radionuclides, or detachable molecule fragments having a typical mass that can be detected in a mass spectrometer. Where said labels are mass labels, some embodiments provide that the labeled amplicons have a single positive or negative net charge, allowing for better delectability in the mass spectrometer. The detection may be carried out and visualized by means of, e.g., matrix assisted laser desorption/ionization mass spectrometry (MALDI) or using electron spray mass spectrometry (ESI).

Methods for isolating DNA suitable for these assay technologies are known in the art. In particular, some embodiments comprise isolation of nucleic acids as described in U.S. patent application Ser. No. 13/470,251 (“Isolation of Nucleic Acids”), incorporated herein by reference in its entirety.

In some embodiments, the markers described herein find use in QuARTS assays performed on stool samples. In some embodiments, methods for producing DNA samples and, in particular, to methods for producing DNA samples that comprise highly purified, low-abundance nucleic acids in a small volume (e.g., less than 100, less than 60 microliters) and that are substantially and/or effectively free of substances that inhibit assays used to test the DNA samples (e.g., PCR, INVADER, QuARTS assays, etc.) are provided. Such DNA samples find use in diagnostic assays that qualitatively detect the presence of, or quantitatively measure the activity, expression, or amount of, a gene, a gene variant (e.g., an allele), or a gene modification (e.g., methylation) present in a sample taken from a patient. For example, some cancers are correlated with the presence of particular mutant alleles or particular methylation states, and thus detecting and/or quantifying such mutant alleles or methylation states has predictive value in the diagnosis and treatment of cancer.

Many valuable genetic markers are present in extremely low amounts in samples and many of the events that produce such markers are rare. Consequently, even sensitive detection methods such as PCR require a large amount of DNA to provide enough of a low-abundance target to meet or supersede the detection threshold of the assay. Moreover, the presence of even low amounts of inhibitory substances compromise the accuracy and precision of these assays directed to detecting such low amounts of a target. Accordingly, provided herein are methods providing the requisite management of volume and concentration to produce such DNA samples.

In some embodiments, the sample comprises stool, tissue sample, an organ secretion, CSF, saliva, blood, or urine. In some embodiments, the subject is human. Such samples can be obtained by any number of means known in the art, such as will be apparent to the skilled person. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens. The technology is not limited in the methods used to prepare the samples and provide a nucleic acid for testing. For example, in some embodiments, a DNA is isolated from a sample (e.g., stool sample, tissue sample, organ secretion sample, CSF sample, saliva sample, blood sample, plasma sample or urine sample) using direct gene capture, e.g., as detailed in U.S. Pat. Nos. 8,808,990 and 9,169,511, and in WO 2012/155072, or by a related method.

The analysis of markers can be carried out separately or simultaneously with additional markers within one test sample. For example, several markers can be combined into one test for efficient processing of multiple samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states over time. Changes in methylation state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events. The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

Genomic DNA may be isolated by any means, including the use of commercially available kits. Briefly, wherein the DNA of interest is encapsulated by a cellular membrane the biological sample must be disrupted and lysed by enzymatic, chemical or mechanical means. The DNA solution may then be cleared of proteins and other contaminants, e.g., by digestion with proteinase K. The genomic DNA is then recovered from the solution. This may be carried out by means of a variety of methods including salting out, organic extraction, or binding of the DNA to a solid phase support. The choice of method will be affected by several factors including time, expense, and required quantity of DNA. All clinical sample types comprising neoplastic matter or pre-neoplastic matter are suitable for use in the present method, e.g., cell lines, histological slides, biopsies, paraffin-embedded tissue, body fluids, stool, tissue, colonic effluent, urine, blood plasma, blood serum, whole blood, isolated blood cells, cells isolated from the blood, and combinations thereof.

The technology is not limited in the methods used to prepare the samples and provide a nucleic acid for testing. For example, in some embodiments, a DNA is isolated from a stool sample or from blood or from a plasma sample using direct gene capture, e.g., as detailed in U.S. Pat. Appl. Ser. No. 61/485,386 or by a related method.

The genomic DNA sample is then treated with at least one reagent, or series of reagents, that distinguishes between methylated and non-methylated CpG dinucleotides within at least one marker comprising a DMR (e.g., DMR 1-38, e.g., as provided by Table 1).

In some embodiments, the reagent converts cytosine bases which are unmethylated at the 5′-position to uracil, thymine, or another base which is dissimilar to cytosine in terms of hybridization behavior. However in some embodiments, the reagent may be a methylation sensitive restriction enzyme.

In some embodiments, the genomic DNA sample is treated in such a manner that cytosine bases that are unmethylated at the 5′ position are converted to uracil, thymine, or another base that is dissimilar to cytosine in terms of hybridization behavior. In some embodiments, this treatment is carried out with bisulfite (hydrogen sulfite, disulfite) followed by alkaline hydrolysis.

The treated nucleic acid is then analyzed to determine the methylation state of the target gene sequences (at least one gene, genomic sequence, or nucleotide from a marker comprising a DMR, e.g., at least one DMR chosen from DMR 1-38, e.g., as provided in Table 1). The method of analysis may be selected from those known in the art, including those listed herein, e.g., QuARTS and MSP as described herein.

Aberrant methylation, more specifically hypermethylation of a marker comprising a DMR (e.g., DMR 1-38, e.g., as provided by Table 1) is associated with multiple types of cancer. Such methods are not limited to the detection for the presence or absence of specific types of cancer. In some embodiments, the types of cancer include, but are not limited to, liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer.

The technology relates to the analysis of any sample associated with multiple types of cancer. For example, in some embodiments the sample comprises a biological fluid obtained from a patient. In some embodiments, the sample comprises a secretion. In some embodiments, the sample comprises blood, serum, plasma, gastric secretions, pancreatic juice, a gastrointestinal biopsy sample, and/or cells recovered from stool. In some embodiments, the subject is human. The sample may include cells, secretions, or tissues from the lymph gland, breast, liver, bile ducts, pancreas, stomach, colon, rectum, esophagus, small intestine, appendix, duodenum, polyps, gall bladder, anus, and/or peritoneum. In some embodiments, the sample comprises cellular fluid, ascites, urine, feces, pancreatic fluid, fluid obtained during endoscopy, blood, mucus, or saliva.

Such samples can be obtained by any number of means known in the art, such as will be apparent to the skilled person. For instance, urine and fecal samples are easily attainable, while blood, ascites, serum, or pancreatic fluid samples can be obtained parenterally by using a needle and syringe, for instance. Cell free or substantially cell free samples can be obtained by subjecting the sample to various techniques known to those of skill in the art which include, but are not limited to, centrifugation and filtration. Although it is generally preferred that no invasive techniques are used to obtain the sample, it still may be preferable to obtain samples such as tissue homogenates, tissue sections, and biopsy specimens.

In some embodiments, the technology relates to a method for treating a patient (e.g., a patient with any type of cancer), the method comprising determining either or both of 1) the methylation state of one or more methylation marker as provided herein, and 2) measuring the expression and/or activity level of one or more protein markers, and administering a treatment to the patient based on the results of determining the methylation state and/or protein marker expression and/or activity level. The treatment may be administration of a pharmaceutical compound, a vaccine, performing a surgery, imaging the patient, performing another test. Preferably, said use is in a method of clinical screening, a method of prognosis assessment, a method of monitoring the results of therapy, a method to identify patients most likely to respond to a particular therapeutic treatment, a method of imaging a patient or subject, and a method for drug screening and development.

In some embodiments of the technology, a method for diagnosing a specific type of cancer in a subject is provided. The terms “diagnosing” and “diagnosis” as used herein refer to methods by which the skilled artisan can estimate and even determine whether or not a subject is suffering from a given disease or condition or may develop a given disease or condition in the future. The skilled artisan often makes a diagnosis on the basis of one or more diagnostic indicators, such as for example one or more biomarkers (e.g., one or more methylated markers, methylated marker genes, genes, DMRs, and/or DNA methylated markers as disclosed herein), the methylation state of which is indicative of the presence, severity, or absence of the condition, and/or the expression and/or activity level of one or more protein markers. Such methods are not limited to the diagnosis of a specific type of cancer. In some embodiments, the types of cancer include, but are not limited to, liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer.

Along with diagnosis, clinical cancer prognosis relates to determining the aggressiveness of the cancer and the likelihood of tumor recurrence to plan the most effective therapy. If a more accurate prognosis can be made or even a potential risk for developing the cancer can be assessed, appropriate therapy, and in some instances less severe therapy for the patient can be chosen. Assessment (e.g., determining methylation state) of cancer biomarkers is useful to separate subjects with good prognosis and/or low risk of developing cancer who will need no therapy or limited therapy from those more likely to develop cancer or suffer a recurrence of cancer who might benefit from more intensive treatments.

As such, “making a diagnosis” or “diagnosing”, as used herein, is further inclusive of determining a risk of developing cancer or determining a prognosis, which can provide for predicting a clinical outcome (with or without medical treatment), selecting an appropriate treatment (or whether treatment would be effective), or monitoring a current treatment and potentially changing the treatment, based on the measure of the diagnostic biomarkers (e.g., DMR) disclosed herein. Further, in some embodiments of the presently disclosed subject matter, multiple determination of the biomarkers over time can be made to facilitate diagnosis and/or prognosis. A temporal change in the biomarker can be used to predict a clinical outcome, monitor the progression of cancer or a subtype of cancer, and/or monitor the efficacy of appropriate therapies directed against the cancer. In such an embodiment for example, one might expect to see a change in the methylation state of one or more biomarkers (e.g., DMR) disclosed herein (and potentially one or more additional biomarker(s), if monitored) and/or expression and/or activity level of a protein marker in a biological sample over time during the course of an effective therapy.

The presently disclosed subject matter further provides in some embodiments a method for determining whether to initiate or continue prophylaxis or treatment of a cancer in a subject. In some embodiments, the method comprises providing a series of biological samples over a time period from the subject; analyzing the series of biological samples to one or both of 1) determine a methylation state of at least one biomarker disclosed herein in each of the biological samples, and 2) measure the expression and/or activity level of one or more protein markers (CEA, CA125, CA19.9, AFP, CA-15-3) in the biological samples; and comparing any measurable change in the methylation states of one or more of the biomarkers and/or protein markers in each of the biological samples. Any changes over the time period can be used to predict risk of developing cancer, predict clinical outcome, determine whether to initiate or continue the prophylaxis or therapy of the cancer, and whether a current therapy is effectively treating the cancer. For example, a first time point can be selected prior to initiation of a treatment and a second time point can be selected at some time after initiation of the treatment. Methylation states and protein marker expression/activity levels can be measured in each of the samples taken from different time points and qualitative and/or quantitative differences noted. A change in the methylation states of the biomarker levels and/or protein marker expression/activity levels from the different samples can be correlated with a specific cancer risk, prognosis, determining treatment efficacy, and/or progression of the cancer in the subject.

In preferred embodiments, the methods and compositions of the invention are for treatment or diagnosis of disease at an early stage, for example, before symptoms of the disease appear. In some embodiments, the methods and compositions of the invention are for treatment or diagnosis of disease at a clinical stage.

As noted, in some embodiments, multiple determinations of one or more diagnostic or prognostic biomarkers can be made, and a temporal change in the marker can be used to determine a diagnosis or prognosis. For example, a diagnostic marker can be determined at an initial time, and again at a second time. In such embodiments, an increase in the marker from the initial time to the second time can be diagnostic of a particular type or severity of cancer, or a given prognosis. Likewise, a decrease in the marker from the initial time to the second time can be indicative of a particular type or severity of cancer, or a given prognosis. Furthermore, the degree of change of one or more markers can be related to the severity of the cancer and future adverse events. The skilled artisan will understand that, while in certain embodiments comparative measurements can be made of the same biomarker at multiple time points, one can also measure a given biomarker at one time point, and a second biomarker at a second time point, and a comparison of these markers can provide diagnostic information.

As used herein, the phrase “determining the prognosis” refers to methods by which the skilled artisan can predict the course or outcome of a condition in a subject. The term “prognosis” does not refer to the ability to predict the course or outcome of a condition with 100% accuracy, or even that a given course or outcome is predictably more or less likely to occur based on the methylation state of a biomarker (e.g., a DMR and/or protein marker). Instead, the skilled artisan will understand that the term “prognosis” refers to an increased probability that a certain course or outcome will occur; that is, that a course or outcome is more likely to occur in a subject exhibiting a given condition, when compared to those individuals not exhibiting the condition. For example, in individuals not exhibiting the condition (e.g., having a normal methylation state of one or more DMR, and/or protein marker expression and/or activity levels), the chance of a given outcome (e.g., suffering from a specific type of cancer) may be very low.

In some embodiments, a statistical analysis associates a prognostic indicator with a predisposition to an adverse outcome. For example, in some embodiments, a methylation state and/or or protein marker expression/activity level different from that in a normal control sample obtained from a patient who does not have a cancer can signal that a subject is more likely to suffer from a cancer than subjects with a level that is more similar to the methylation state in the control sample, as determined by a level of statistical significance. Additionally, a change in methylation state and/or or protein marker expression/activity level from a baseline (e.g., “normal”) level can be reflective of subject prognosis, and the degree of change in methylation state and/or or protein marker expression/activity level can be related to the severity of adverse events. Statistical significance is often determined by comparing two or more populations and determining a confidence interval and/or ap value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001.

In other embodiments, a threshold degree of change in the methylation state and/or or protein marker expression/activity level of a prognostic or diagnostic biomarker disclosed herein (e.g., a DMR; protein marker) can be established, and the degree of change in the methylation state and/or or protein marker expression/activity level of the biomarker in a biological sample is simply compared to the threshold degree of change in the methylation state and/or or protein marker expression/activity level. A preferred threshold change in the methylation state and/or or protein marker expression/activity level for biomarkers provided herein is about 5%, about 10%, about 15%, about 20%, about 25%, about 30%, about 50%, about 75%, about 100%, and about 150%. In yet other embodiments, a “nomogram” can be established, by which a methylation state and/or or protein marker expression/activity level of a prognostic or diagnostic indicator (biomarker or combination of biomarkers) is directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

In some embodiments, a control sample is analyzed concurrently with the biological sample, such that the results obtained from the biological sample can be compared to the results obtained from the control sample. Additionally, it is contemplated that standard curves can be provided, with which assay results for the biological sample may be compared. Such standard curves present methylation states and/or or protein marker expression/activity level states of a biomarker as a function of assay units, e.g., fluorescent signal intensity, if a fluorescent label is used. Using samples taken from multiple donors, standard curves can be provided for control methylation states of the one or more biomarkers in normal tissue, as well as for “at-risk” levels of the one or more biomarkers in plasma taken from donors with a specific type of cancer. In certain embodiments of the method, a subject is identified as having cancer upon identifying an aberrant methylation state of one or more DMR and/or or protein marker expression/activity level provided herein in a biological sample obtained from the subject. In other embodiments of the method, the detection of an aberrant methylation state and/or or protein marker expression/activity level state of one or more of such biomarkers in a biological sample obtained from the subject results in the subject being identified as having cancer.

The analysis of markers can be carried out separately or simultaneously with additional markers within one test sample. For example, several markers can be combined into one test for efficient processing of a multiple of samples and for potentially providing greater diagnostic and/or prognostic accuracy. In addition, one skilled in the art would recognize the value of testing multiple samples (for example, at successive time points) from the same subject. Such testing of serial samples can allow the identification of changes in marker methylation states and/or protein marker expression/activity level states over time. Changes in methylation state and/or protein marker expression/activity level state, as well as the absence of change in methylation state, can provide useful information about the disease status that includes, but is not limited to, identifying the approximate time from onset of the event, the presence and amount of salvageable tissue, the appropriateness of drug therapies, the effectiveness of various therapies, and identification of the subject's outcome, including risk of future events.

The analysis of biomarkers can be carried out in a variety of physical formats. For example, the use of microtiter plates or automation can be used to facilitate the processing of large numbers of test samples. Alternatively, single sample formats could be developed to facilitate immediate treatment and diagnosis in a timely fashion, for example, in ambulatory transport or emergency room settings.

In some embodiments, the subject is diagnosed as having a specific type of cancer if, when compared to a control methylation state and/or or protein marker expression/activity level state, there is a measurable difference in the methylation state and/or or protein marker expression/activity level of at least one biomarker in the sample. Conversely, when no change in methylation state and/or or protein marker expression/activity level state is identified in the biological sample, the subject can be identified as not having a specific type of cancer, not being at risk for the cancer, or as having a low risk of the cancer. In this regard, subjects having the cancer or risk thereof can be differentiated from subjects having low to substantially no cancer or risk thereof. Those subjects having a risk of developing a specific type of cancer can be placed on a more intensive and/or regular screening schedule. On the other hand, those subjects having low to substantially no risk may avoid being subjected to additional testing for cancer risk (e.g., invasive procedure), until such time as a future screening, for example, a screening conducted in accordance with the present technology, indicates that a risk of cancer risk has appeared in those subjects.

As mentioned above, depending on the embodiment of the method of the present technology, detecting a change in methylation state and/or protein marker expression/activity level state of the one or more biomarkers can be a qualitative determination or it can be a quantitative determination. As such, the step of diagnosing a subject as having, or at risk of developing, a specific type of cancer indicates that certain threshold measurements are made, e.g., the methylation state and/or protein marker expression/activity level state of the one or more biomarkers in the biological sample varies from a predetermined control methylation state and/or control protein marker expression/activity level state. In some embodiments of the method, the control methylation state is any detectable methylation state of the biomarker. In some embodiments, the control protein marker expression/activity level state is any measurable and/or or protein marker expression/activity level state of the protein marker. In other embodiments of the method where a control sample is tested concurrently with the biological sample, the predetermined methylation state is the methylation state in the control sample, and the predetermined protein marker expression/activity level control state is the and/or protein marker expression/activity level state in the control sample. In other embodiments of the method, the predetermined methylation state and/or predetermined protein marker expression/activity level state is based upon and/or identified by a standard curve. In other embodiments of the method, the predetermined methylation state and/or predetermined protein marker expression/activity level state is a specifically state or range of state. As such, the predetermined methylation state and/or predetermined protein marker expression/activity level state can be chosen, within acceptable limits that will be apparent to those skilled in the art, based in part on the embodiment of the method being practiced and the desired specificity, etc.

Further with respect to diagnostic methods, a preferred subject is a vertebrate subject. A preferred vertebrate is warm-blooded; a preferred warm-blooded vertebrate is a mammal. A preferred mammal is most preferably a human. As used herein, the term “subject’ includes both human and animal subjects. Thus, veterinary therapeutic uses are provided herein. As such, the present technology provides for the diagnosis of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Thus, also provided is the diagnosis and treatment of livestock, including, but not limited to, domesticated swine, ruminants, ungulates, horses (including race horses), and the like.

In certain embodiments, the technology provides steps for reacting a nucleic acid comprising a DMR with a reagent capable of modifying nucleic acid in a methylation-specific manner (e.g., a methylation-sensitive restriction enzyme, a methylation-dependent restriction enzyme, and a bisulfite reagent) (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), borane reducing agent) to produce, for example, nucleic acid modified in a methylation-specific manner; sequencing the nucleic acid modified in a methylation-specific manner to provide a nucleotide sequence of the nucleic acid modified in a methylation-specific manner; comparing the nucleotide sequence of the nucleic acid modified in a methylation-specific manner with a nucleotide sequence of a nucleic acid comprising the DMR from a subject who does not have a specific type of cancer to identify differences in the two sequences; and identifying the subject as having a specific type of cancer when a difference is present. In some embodiments, the cancer is any type of cancer. In some embodiments, the cancer is selected from liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer.

The technology further provides compositions. In certain embodiments, the technology provides composition comprising a nucleic acid comprising a DMR and a bisulfite reagent In certain embodiments, composition comprising a nucleic acid comprising a DMR and one or more oligonucleotide according to SEQ ID NOS 1-126 are provided. In certain embodiments, compositions comprising a nucleic acid comprising a DMR and a methylation-sensitive restriction enzyme are provided. In certain embodiments, compositions comprising a nucleic acid comprising a DMR and a polymerase are provided.

The technology further provides kits. The kits comprise embodiments of the compositions, devices, apparatuses, etc. described herein, and instructions for use of the kit. Such instructions describe appropriate methods for preparing an analyte from a sample, e.g., for collecting a sample and preparing a nucleic acid from the sample. Individual components of the kit are packaged in appropriate containers and packaging (e.g., vials, boxes, blister packs, ampules, jars, bottles, tubes, and the like) and the components are packaged together in an appropriate container (e.g., a box or boxes) for convenient storage, shipping, and/or use by the user of the kit. It is understood that liquid components (e.g., a buffer) may be provided in a lyophilized form to be reconstituted by the user. Kits may include a control or reference for assessing, validating, and/or assuring the performance of the kit. For example, a kit for assaying the amount of a nucleic acid present in a sample may include a control comprising a known concentration of the same or another nucleic acid for comparison and, in some embodiments, a detection reagent (e.g., a primer) specific for the control nucleic acid. The kits are appropriate for use in a clinical setting and, in some embodiments, for use in a user's home. The components of a kit, in some embodiments, provide the functionalities of a system for preparing a nucleic acid solution from a sample. In some embodiments, certain components of the system are provided by the user.

In certain embodiments, the technology is related to embodiments of compositions (e.g., reaction mixtures). In some embodiments are provided a composition comprising a nucleic acid comprising a DMR and a reagent capable of modifying DNA in a methylation-specific manner (e.g., a methylation-sensitive restriction enzyme, a methylation-dependent restriction enzyme, and a bisulfate reagent) (e.g., a methylation-sensitive restriction enzyme, a methylation-dependent restriction enzyme, Ten Eleven Translocation (TET) enzyme (e.g., human TET1, human TET2, human TET3, murine TET1, murine TET2, murine TET3, Naegleria TET (NgTET), Coprinopsis cinerea (CcTET)), or a variant thereof), borane reducing agent). Some embodiments provide a composition comprising a nucleic acid comprising a DMR and an oligonucleotide as described herein. Some embodiments provide a composition comprising a nucleic acid comprising a DMR and a methylation-sensitive restriction enzyme. Some embodiments provide a composition comprising a nucleic acid comprising a DMR and a polymerase.

In some embodiments, the technology described herein is associated with a programmable machine designed to perform a sequence of arithmetic or logical operations as provided by the methods described herein. For example, some embodiments of the technology are associated with (e.g., implemented in) computer software and/or computer hardware. In one aspect, the technology relates to a computer comprising a form of memory, an element for performing arithmetic and logical operations, and a processing element (e.g., a microprocessor) for executing a series of instructions (e.g., a method as provided herein) to read, manipulate, and store data. In some embodiments, a microprocessor is part of a system for determining a methylation state (e.g., of one or more DMR, e.g., DMR 1-38 as provided in Table 1); comparing methylation states; generating standard curves; determining a Ct value; calculating a fraction, frequency, or percentage of methylation; identifying a CpG island; determining a specificity and/or sensitivity of an assay or marker; calculating an ROC curve and an associated AUC; sequence analysis; all as described herein or is known in the art. In some embodiments, a microprocessor is part of a system for determining a level of protein expression and/or activity (e.g., one or more protein markers described herein); comparing level of protein marker expression or activity in comparison to a standard non-cancerous level; all as described herein or is known in the art. In some embodiments, a microprocessor is part of a system for 1) determining a methylation state (e.g., of one or more DMR, e.g., DMR 1-38 as provided in Table 1); comparing methylation states; generating standard curves; determining a Ct value; calculating a fraction, frequency, or percentage of methylation; identifying a CpG island; determining a specificity and/or sensitivity of an assay or marker; calculating an ROC curve and an associated AUC; sequence analysis; all as described herein or is known in the art; and 2) determining a level of protein expression and/or activity (e.g., one or more protein markers described herein); comparing level of protein marker expression or activity in comparison to a standard non-cancerous level; all as described herein or is known in the art.

In some embodiments, a software or hardware component receives the results of multiple assays and determines a single value result to report to a user that indicates a cancer risk based on the results of the multiple assays (e.g., determining the methylation state of multiple DMR, e.g., as provided in Table 1) (e.g., determining protein marker expression and/or activity levels). Related embodiments calculate a risk factor based on a mathematical combination (e.g., a weighted combination, a linear combination) of the results from the multiple assays (e.g., determining the methylation state of multiple DMR, e.g., as provided in Table 1) (e.g., determining protein marker expression and/or activity levels). In some embodiments, the methylation state of a DMR defines a dimension and may have values in a multidimensional space and the coordinate defined by the methylation states of multiple DMR is a result, e.g., to report to a user, e.g., related to a cancer risk.

In some embodiments, the technology provided herein is associated with a plurality of programmable devices that operate in concert to perform a method as described herein. For example, in some embodiments, a plurality of computers (e.g., connected by a network) may work in parallel to collect and process data, e.g., in an implementation of cluster computing or grid computing or some other distributed computer architecture that relies on complete computers (with onboard CPUs, storage, power supplies, network interfaces, etc.) connected to a network (private, public, or the internet) by a conventional network interface, such as Ethernet, fiber optic, or by a wireless network technology.

For example, some embodiments provide a computer that includes a computer-readable medium. The embodiment includes a random access memory (RAM) coupled to a processor. The processor executes computer-executable program instructions stored in memory. Such processors may include a microprocessor, an ASIC, a state machine, or other processor, and can be any of a number of computer processors, such as processors from Intel Corporation of Santa Clara, Calif. and Motorola Corporation of Schaumburg, Ill. Such processors include, or may be in communication with, media, for example computer-readable media, which stores instructions that, when executed by the processor, cause the processor to perform the steps described herein.

Computers are connected in some embodiments to a network. Computers may also include a number of external or internal devices such as a mouse, a CD-ROM, DVD, a keyboard, a display, or other input or output devices. Examples of computers are personal computers, digital assistants, personal digital assistants, cellular phones, mobile phones, smart phones, pagers, digital tablets, laptop computers, internet appliances, and other processor-based devices. In general, the computers related to aspects of the technology provided herein may be any type of processor-based platform that operates on any operating system, such as Microsoft Windows, Linux, UNIX, Mac OS X, etc., capable of supporting one or more programs comprising the technology provided herein. Some embodiments comprise a personal computer executing other application programs (e.g., applications). The applications can be contained in memory and can include, for example, a word processing application, a spreadsheet application, an email application, an instant messenger application, a presentation application, an Internet browser application, a calendar/organizer application, and any other application capable of being executed by a client device.

All such components, computers, and systems described herein as associated with the technology may be logical or virtual.

In certain embodiments, the technology provides systems for screening for multiple types of cancer in a sample obtained from a subject are provided by the technology. Exemplary embodiments of systems include, e.g., a system for screening for multiple types of cancer in a sample obtained from a subject (e.g., a stool sample, a tissue sample, an organ secretion sample, a CSF sample, a saliva sample, a blood sample, a plasma sample, or a urine sample), the system comprising:
an analysis component configured to one or both of determining the methylation state of one or more methylated markers in a sample and determining the expression and/or activity level of one or more protein markers in the sample,
a software component configured to compare the methylation state of the one or more methylated markers in the sample and/or expression and/or activity level of the one or more protein markers in the sample with a control sample or a reference sample recorded in a database, and
an alert component configured to alert a user of a cancer associated state.

In some embodiments, an alert is determined by a software component that receives the results from multiple assays (e.g., determining the methylation states of the one or more methylated markers) (e.g., determining the expression and/or activity level of the one or more protein markers) and calculating a value or result to report based on the multiple results.

Some embodiments provide a database of weighted parameters associated with each methylated marker and/or protein marker expression and/or activity level provided herein for use in calculating a value or result and/or an alert to report to a user (e.g., such as a physician, nurse, clinician, etc.). In some embodiments all results from multiple assays are reported. In some embodiments, one or more results are used to provide a score, value, or result based on a composite of one or more results from multiple assays that is indicative of a cancer risk in a subject. Such methods are not limited to particular methylation markers.

In such methods and systems, the one or more methylation markers comprise a base in a DMR selected from a group consisting of DMR 1-38 as provided in Table 1.

In this detailed description of the various embodiments, for purposes of explanation, numerous specific details are set forth to provide a thorough understanding of the embodiments disclosed. One skilled in the art will appreciate, however, that these various embodiments may be practiced with or without these specific details. In other instances, structures and devices are shown in block diagram form. Furthermore, one skilled in the art can readily appreciate that the specific sequences in which methods are presented and performed are illustrative and it is contemplated that the sequences can be varied and still remain within the spirit and scope of the various embodiments disclosed herein.

### EXPERIMENTAL

The following examples are illustrative, but not limiting, of the present invention. Other suitable modifications and adaptations of the variety of conditions and parameters normally encountered in clinical therapy and which are obvious to those skilled in the art are within the spirit and scope of the invention. As used herein, terms such as “our”, “we”, “I”, and similar terms, refers to the inventive entity for the inventions described herein.

### Example I

This example describes experiments conducted to assess the feasibility of targeted assay of a panel of methylated DNA markers (MDMs) and proteins for detection of highly lethal cancers.

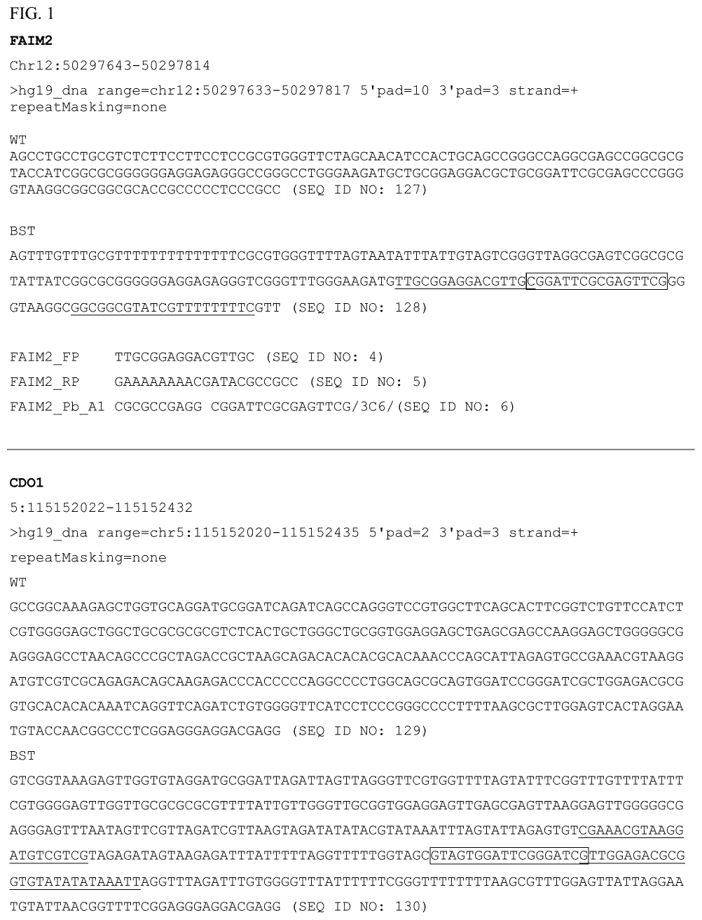

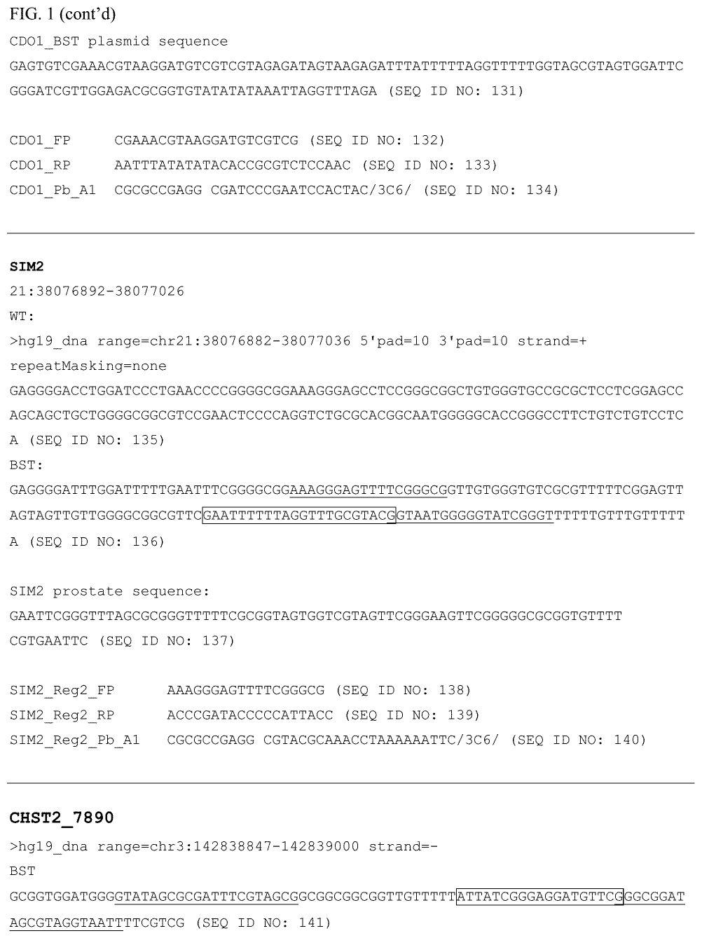

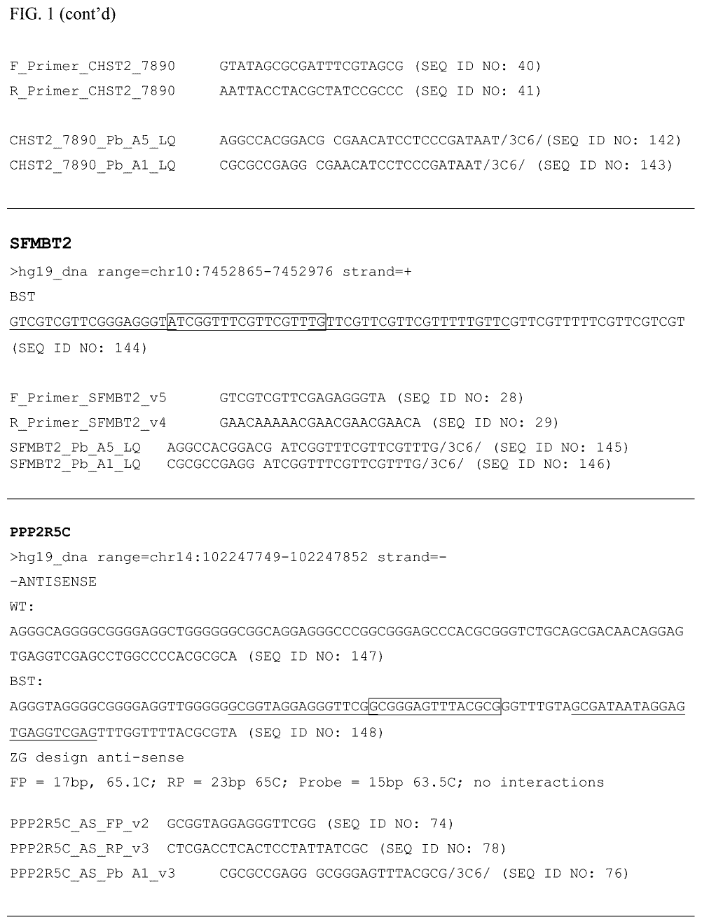

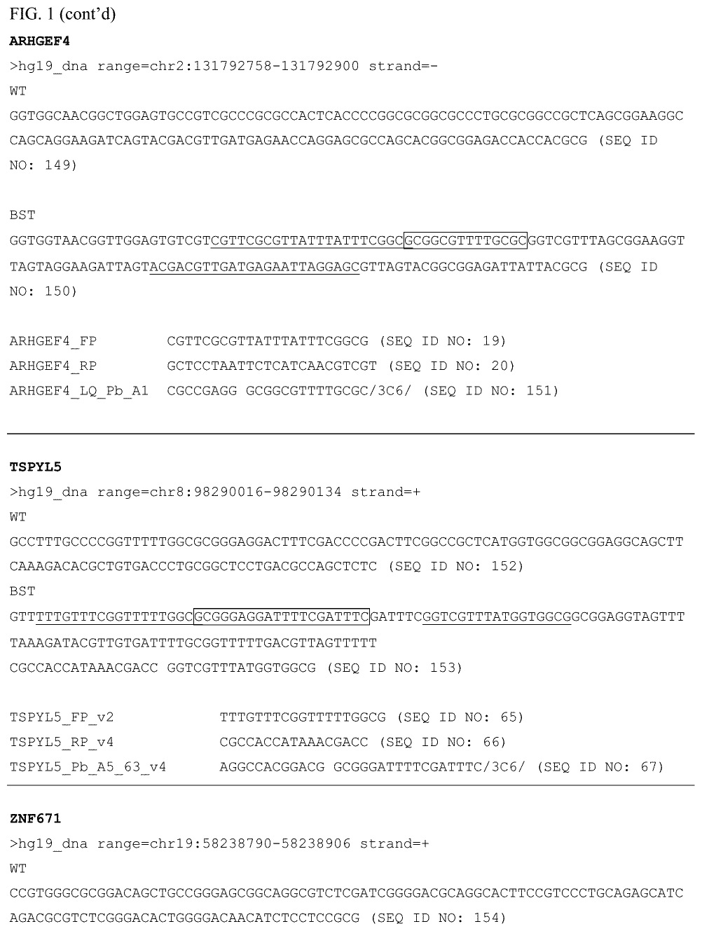

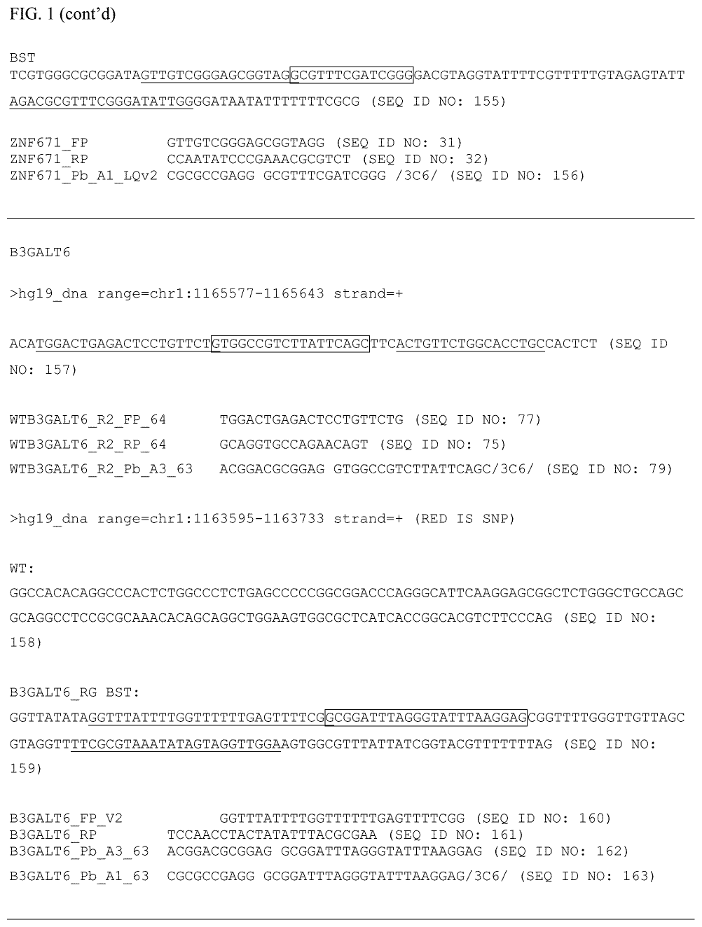

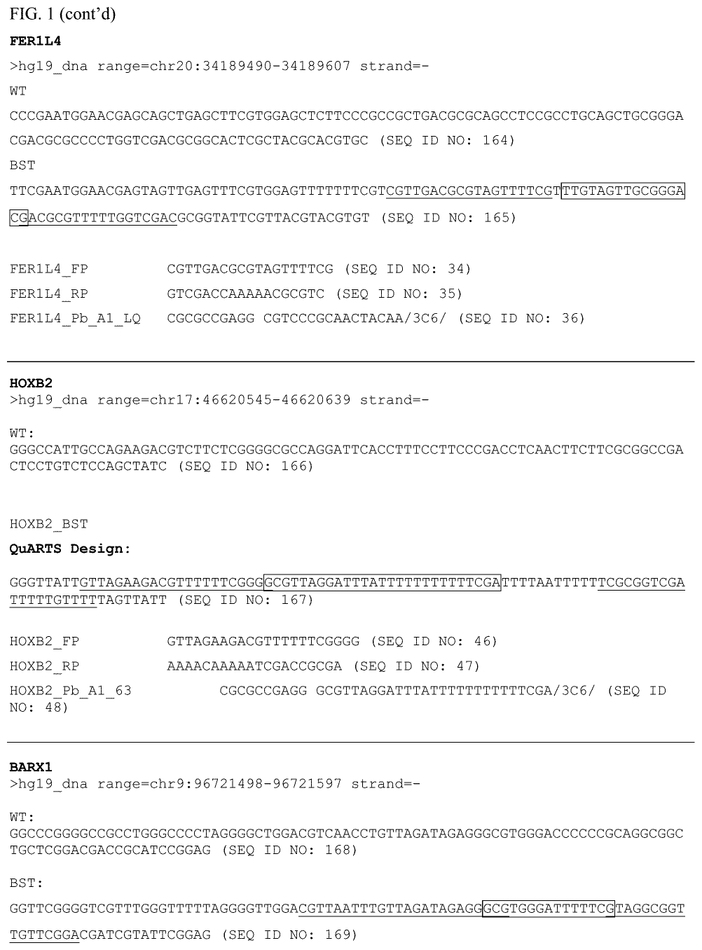

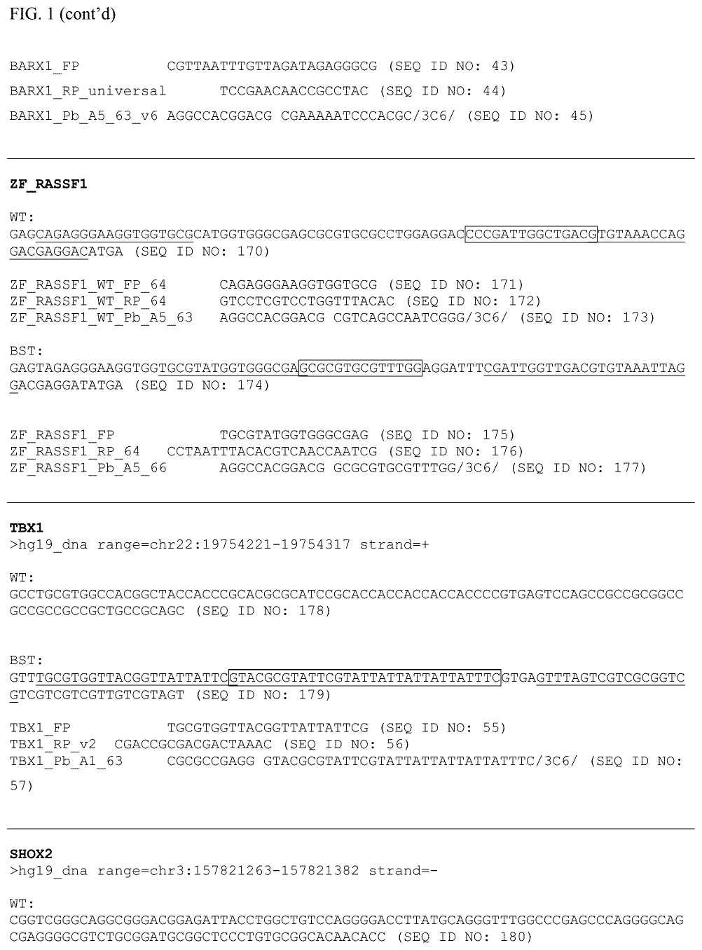

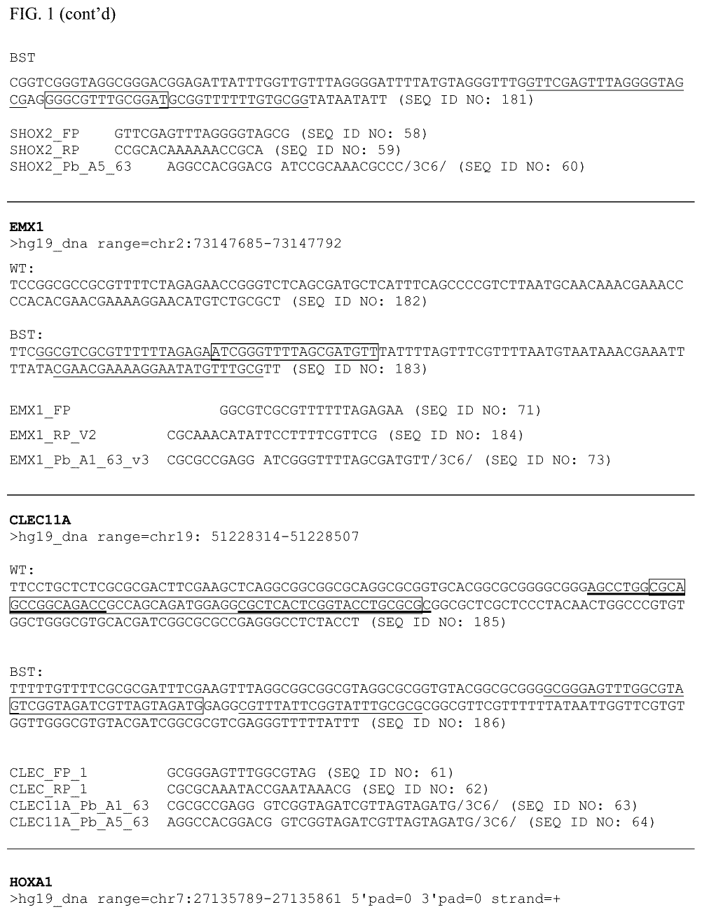

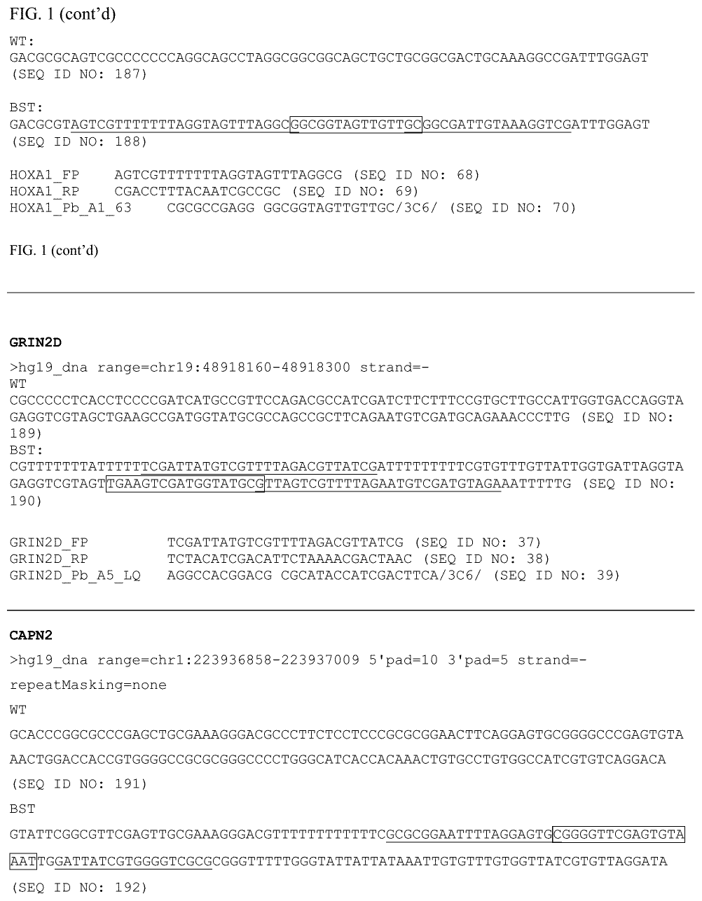

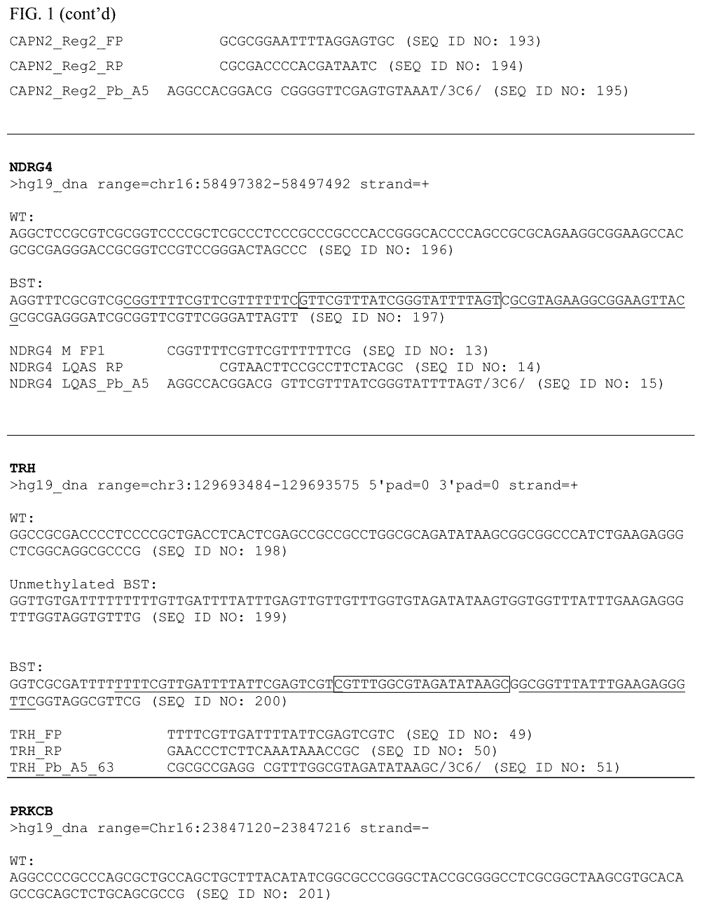

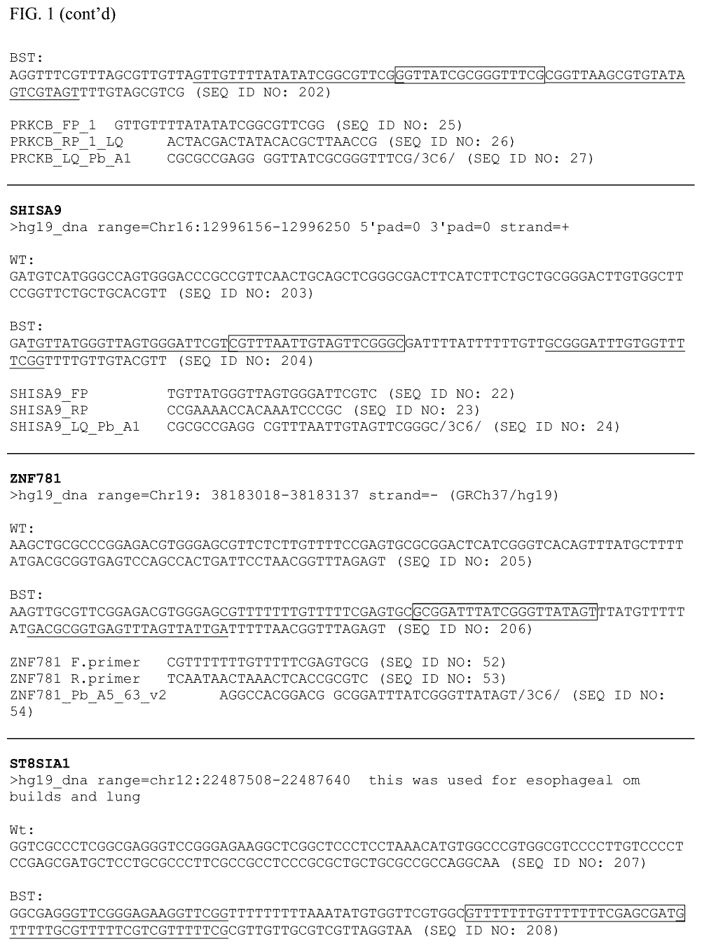

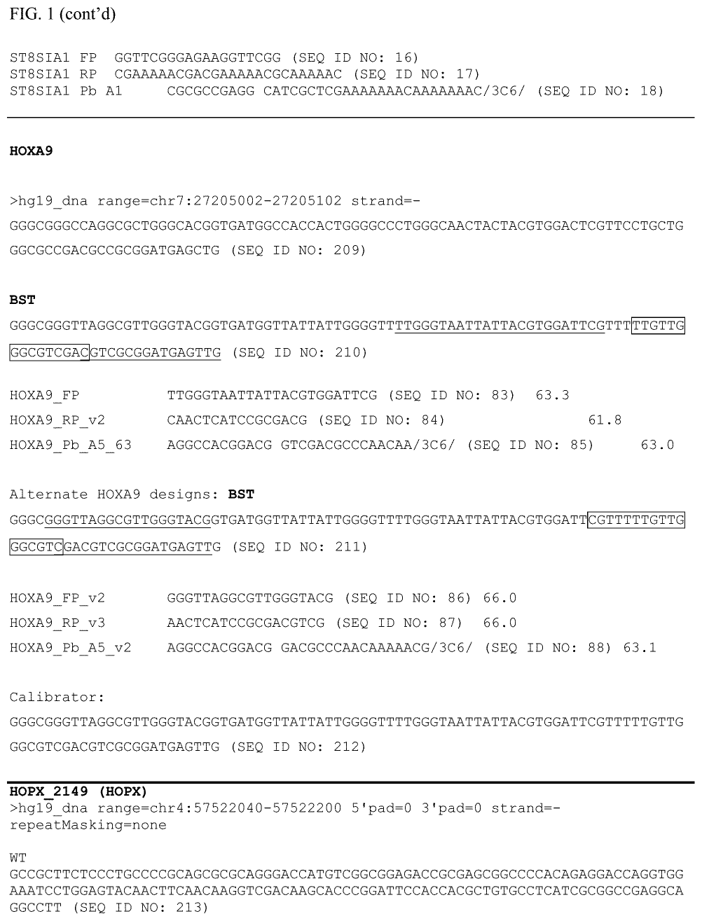

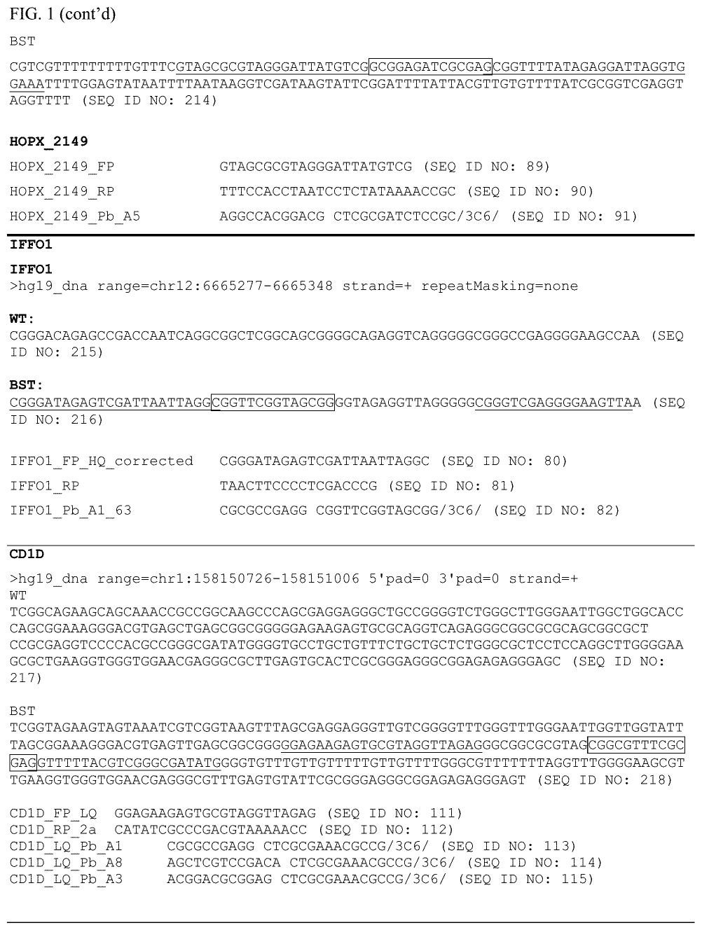

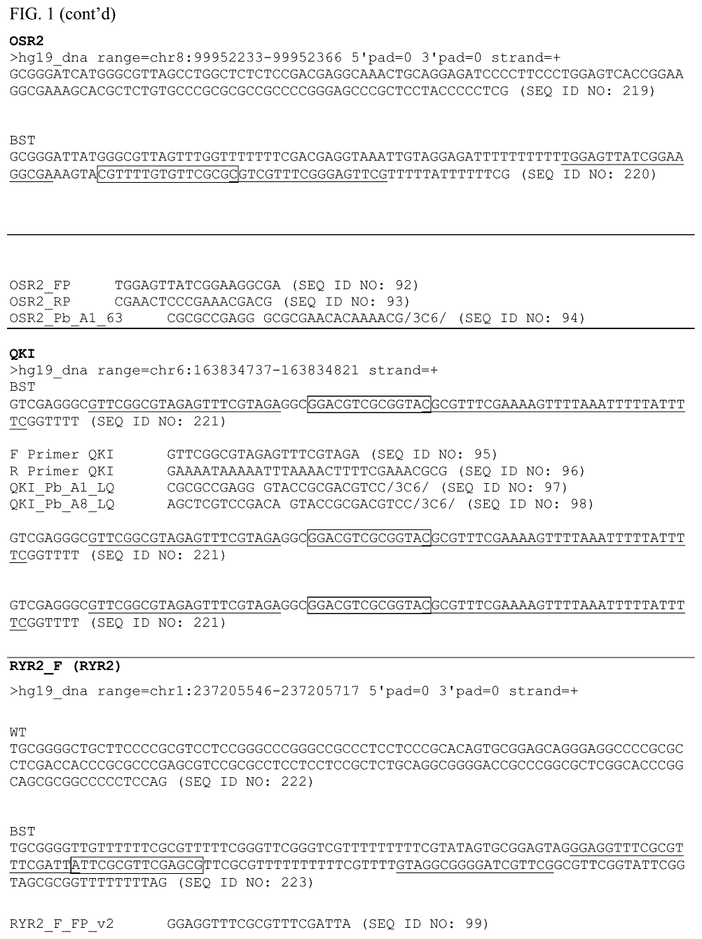

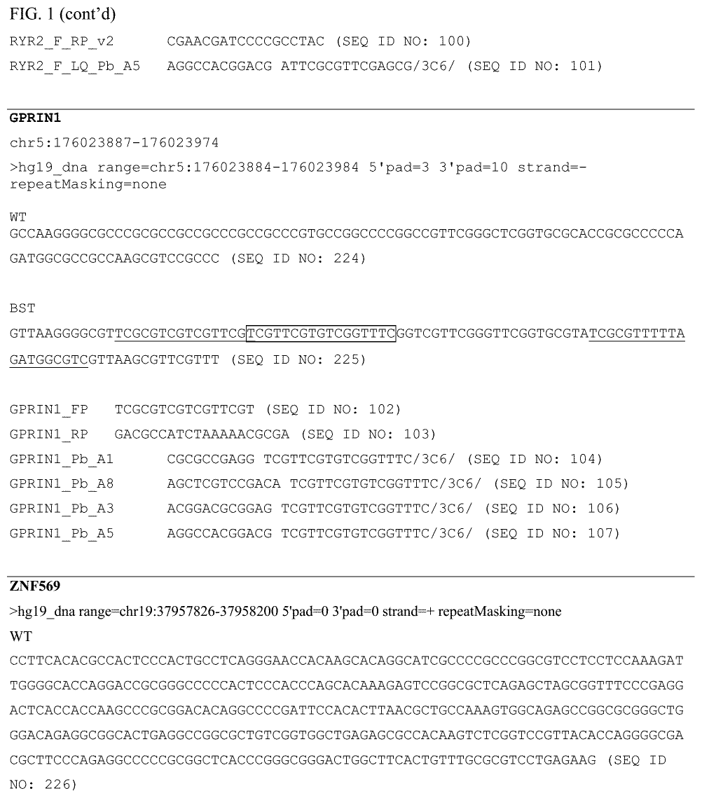

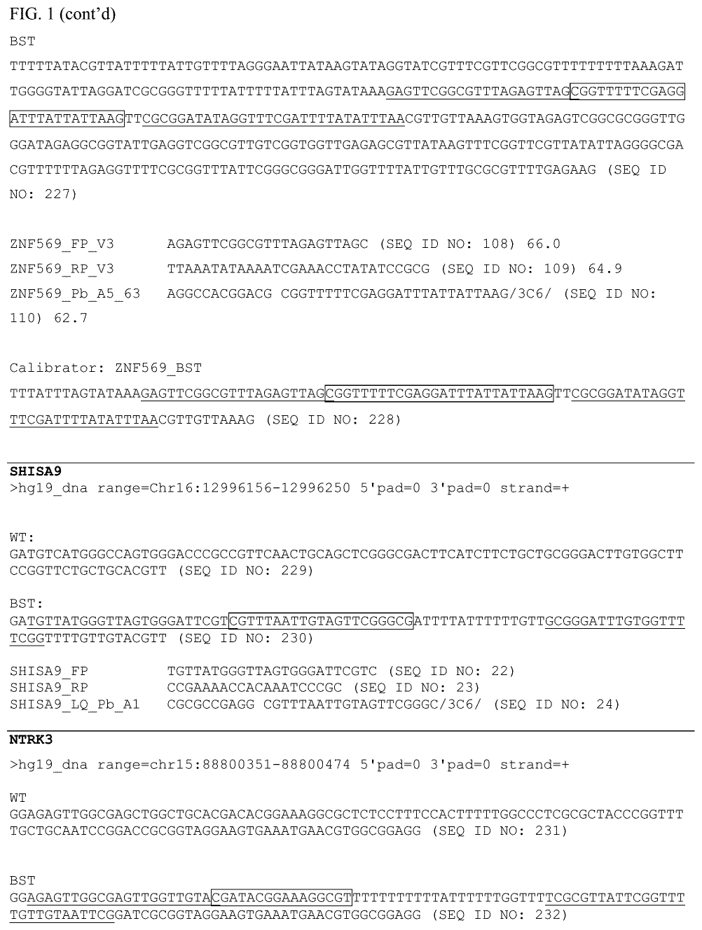

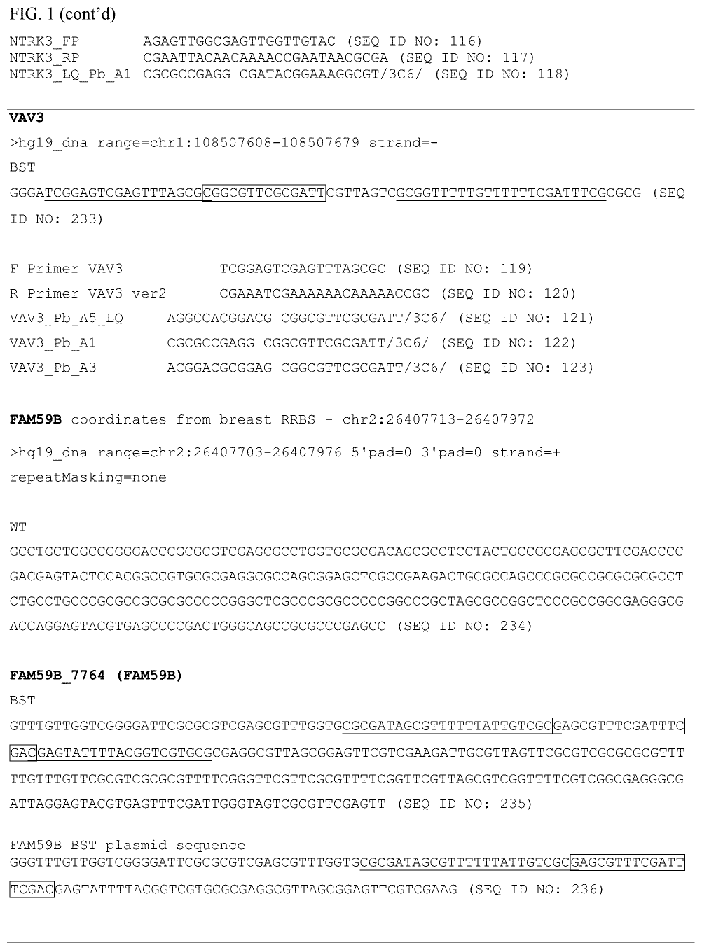

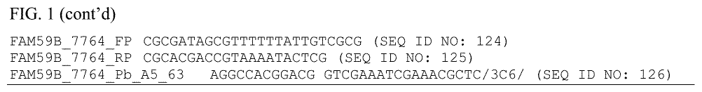

Prospectively collected plasmas from 180 cases of 6 cancer types (stage A-D liver (n=36), and TNM stage II-IV esophageal (n=18), lung (n=36), ovarian (n=30), pancreatic (n=30), and stomach (n=30)) and 257 smoking status, age-, and gender-matched asymptomatic controls were tested in blinded fashion. Using multiplex PCR followed by LQAS (Long probe Quantitative Amplified Signal) assay (see, e.g., WO2017/075061 and U.S. patent application Ser. No. 15/841,006 for general techniques), a post-bisulfate quantification of 38 MDMs, previously found to be common among many cancers, on DNA extracted from 3 mL of plasma was performed (see, Table 1 (the genomic coordinates for the regions shown in Table 1 are based on the Human February 2009 (GRCh37/hg19) Assembly) and FIG. 1; Table 2 and FIG. 1 show the primer and probe sequences for the markers shown in Table 1).

TABLE 1

| | | Region on Chromosome |
|---|---|---|
| DMR No. | Gene Annotation | (starting base-ending base) |
| 1 | FAIM2 | chr12: 50297633-50297817 |
| 2 | CDO1 | chr5: 115152020-115152435 |
| 3 | SIM2 | chr21: 38076882-38077036 |
| 4 | CHST2_7890 | chr3: 142838847-142839000 |
| 5 | SFMBT2 | chr10: 7452865-7452976 |
| 6 | PPP2R5C | chr14: 102247749-102247852 |
| 7 | ARHGEF4 | chr2: 131792758-131792900 |
| 8 | TSPYL5 | chr8: 98290016-98290134 |
| 9 | ZNF671 | chr19: 58238790-58238906 |
| 10 | B3GALT6 | chr1: 1165577-1165643 |
| 11 | FER1L4 | chr20: 34189490-34189607 |
| 12 | HOXB2 | chr17: 46620545-46620639 |
| 13 | BARX1 | chr9: 96721498-96721597 |
| 14 | TBX1 | chr22: 19754221-19754317 |
| 15 | SHOX2 | chr3: 157821263-157821382 |
| 16 | EMX1 | chr2: 73147685-73147792 |
| 17 | CLEC11A | chr19: 51228314-51228507 |
| 18 | HOXA1 | chr7: 27135789-27135861 |
| 19 | GRIN2D | chr19: 48918160-48918300 |
| 20 | CAPN2 | chr1: 223936858-223937009 |
| 21 | NDRG4 | chr16: 58497382-58497492 |
| 22 | TRH | chr3: 129693484-129693575 |
| 23 | PRKCB | chr16: 12996156-12996250 |
| 24 | SHISA9 | Chr16: 12996156-12996250 |
| 25 | ZNF781 | chr19: 38183018-38183137 |
| 26 | ST8SIA1 | chr12: 22487508-22487640 |
| 27 | IFFO1 | chr12: 6665277-6665348 |
| 28 | HOXA9 | chr7: 27205002-27205102 |
| 29 | HOPX | chr4: 57522040-57522200 |
| 30 | OSR2 | chr8: 99952233-99952366 |
| 31 | QKI | chr6: 163834737-163834821 |
| 32 | RYR2 | chr1: 237205546-237205717 |
| 33 | GPRIN1 | chr5: 176023887-176023974 |
| 34 | ZNF569 | chr19: 37957826-37958200 |
| 35 | CD1D | chr1: 158150726-158151006 |
| 36 | NTRK3 | chr15: 88800351-88800474 |
| 37 | VAV3 | chr1: 108507608-108507679 |
| 38 | FAM59B | chr2: 26407703-26407976 |

TABLE 2

| | | | | SEQ |
|---|---|---|---|---|
| DMR | | | | ID |
| No. | Marker | | Sequence | NO: |
| 2 | CDO1 | Forward | CGA AAC GTA AGG ATG TCG TCG | 1 |
| | | Primer | | |
| | | Reverse | AAT TTA TAT ATA CAC CGC GTC | 2 |
| | | Primer | TCC AAC | |
| | | Probe | CGC GCC GAG GCG ATC CCG AAT | 3 |
| | | | CCA CTA C/3C6/ | |
| 1 | FAIM2 | Forward | TTGCGGAGGACGTTGC | 4 |
| | | Primer | | |
| | | Reverse | GAAAAAAAACGATACGCCGCC | 5 |
| | | Primer | | |
| | | Probe | CGCGCCGAGGCGGATTCGCGAGTT | 6 |
| | | | CG/3C6/ | |
| 20 | CAPN2 | Forward | GCG CGG AAT TTT AGG AGT GC | 7 |
| | | Primer | | |
| | | Reverse | CGC GAC CCC ACG ATA ATC | 8 |
| | | Primer | | |
| | | Probe | AGG CCA CGG ACG CGG GGT TCG | 9 |
| | | | AGT GTA AAT/3C6/ | |
| 3 | SIM2 | Forward | AAA GGG AGT TTT CGG GCG | 10 |
| | | Primer | | |
| | | Reverse | ACC CGA TAC CCC CAT TAC C | 11 |
| | | Primer | | |
| | | Probe | CGC GCC GAG GCG TAC GCA AAC | 12 |
| | | | CTA AAA AAT TC/3C6/ | |
| 21 | NDRG4 | Forward | CGGTTTTCGTTCGTTTTTTCG | 13 |
| | | Primer | | |
| | | Reverse | CGTAACTTCCGCCTTCTACGC | 14 |
| | | Primer | | |
| | | Probe | AGGCCACGGACG | 15 |
| | | | GTTCGTTTATCGGGTATTTTAG | |
| | | | T/3C6/ | |
| 26 | STASIA1 | Forward | GGTTCGGGAGAAGGTTCGG | 16 |
| | | Primer | | |
| | | Reverse | CGAAAAACGACGAAAAACGCAAAA | 17 |
| | | Primer | AC | |
| | | Probe | CGCGCCGAGG | 18 |
| | | | CATCGCTCGAAAAAAACAAAAAAA | |
| | | | C/3C6/ | |
| 7 | ARHGEF4 | Forward | CGTTCGCGTTATTTATTTCGGCG | 19 |
| | | Primer | | |
| | | Reverse | GCTCCTAATTCTCATCAACGTCGT | 20 |
| | | Primer | | |
| | | Probe | CGCGCCGAGG | 21 |
| | | | GCGGCGTTTTGCGC/3C6/ | |
| 24 | SHISA9 | Forward | TGTTATGGGTTAGTGGGATTCGTC | 22 |
| | | Primer | | |
| | | Reverse | CCGAAAACCACAAATCCCGC | 23 |
| | | Primer | CGCGCCGAGG | 24 |
| | | Probe | CGTTTAATTGTAGTTCGGGC/3C6/ | |
| 23 | PRKCB | Forward | GTTGTTTTATATATCGGCGTTCGG | 25 |
| | | Primer | | |
| | | Reverse | ACTACGACTATACACGCTTAACCG | 26 |
| | | Primer | CGCGCCGAGG | 27 |
| | | Probe | GGTTATCGCGGGTTTCG/3C6/ | |
| 5 | SFMBT2 | Forward | GTCGTCGTTCGAGAGGGTA | 28 |
| | | Primer | | |
| | | Reverse | GAACAAAAACGAACGAACGAACA | 29 |
| | | Primer | | |
| | | Probe | CGCGCCGAGG | 30 |
| | | | ATCGGTTTCGTTCGTTTG/3C6/ | |
| 9 | ZNF671 | Forward | GTTGTCGGGAGCGGTAGG | 31 |
| | | Primer | | |
| | | Reverse | CCAATATCCCGAAACGCGTCT | 32 |
| | | Primer | | |
| | | Probe | CGCGCCGAGGGCGTTTCGATCGG | 33 |
| | | | G/3C6/ | |
| 11 | FER1L4 | Forward | CGTTGACGCGTAGTTTTCG | 34 |
| | | Primer | | |
| | | Reverse | GTCGACCAAAAACGCGTC | 35 |
| | | Primer | CGCGCCGAGG | 36 |
| | | Probe | CGTCCCGCAACTACAA/3C6/ | |
| 19 | GRIN2D | Forward | TCGATTATGTCGTTTTAGACGTTAT | 37 |
| | | Primer | CG | |
| | | Reverse | TCTACATCGACATTCTAAAACGACT | 38 |
| | | Primer | AAC | |
| | | Probe | AGGCCACGGACG | 39 |
| | | | CGCATACCATCGACTTCA/3C6/ | |
| 4 | CHST2_ | Forward | GTATAGCGCGATTTCGTAGCG | 40 |
| | 7890 | Primer | | |
| | | Reverse | AATTACCTACGCTATCCGCCC | 41 |
| | | Primer | | |
| | | Probe | AGGCCACGGACGCG AAC ATC | 42 |
| | | | CTC CCG ATA AT/3C6/ | |
| 13 | BARX1 | Forward | CGTTAATTTGTTAGATAGAGGGCG | 43 |
| | | Primer | | |
| | | Reverse | TCCGAACAACCGCCTAC | 44 |
| | | Primer | | |
| | | Probe | AGGCCACGGACG | 45 |
| | | | CGAAAAAT000ACGC/3C6/ | |
| 12 | HOXB2 | Forward | GTTAGAAGACGTTTTTTCGGGG | 46 |
| | | Primer | | |
| | | Reverse | AAAACAAAAATCGACCGCGA | 47 |
| | | Primer | | |
| | | Probe | CGCGCCGAGG | 48 |
| | | | GCGTTAGGATTTATTTTTTTTTTTCG | |
| | | | A/3C6/ | |
| 22 | TRH | Forward | TTTTCGTTGATTTTATTCGAGTCGTC | 49 |
| | | Primer | | |
| | | Reverse | GAACCCTCTTCAAATAAACCGC | 50 |
| | | Primer | | |
| | | Probe | CGCGCCGAGG | 51 |
| | | | CGTTTGGCGTAGATATAAGC/3C6/ | |
| 25 | ZNF781 | Forward | CGTTTTTTTGTTTTTCGAGTGCG | 52 |
| | | Primer | | |
| | | Reverse | TCAATAACTAAACTCACCGCGTC | 53 |
| | | Primer | | |
| | | Probe | AGGCCACGGACG | 54 |
| | | | GCGGATTTATCGGGTTATAGT/3C6/ | |
| 14 | TBX1 | Forward | TGCGTGGTTACGGTTATTATTCG | 55 |
| | | Primer | | |
| | | Reverse | CGACCGCGACGACTAAAC | 56 |
| | | Primer | | |
| | | Probe | CGCGCCGAGG | 57 |
| | | | GTACGCGTATTCGTATTATTATTATT | |
| | | | ATTTC/3C6/ | |
| 15 | SHOX2 | Forward | GTTCGAGTTTAGGGGTAGCG | 58 |
| | | Primer | | |
| | | Reverse | CCGCACAAAAAACCGCA | 59 |
| | | Primer | | |
| | | Probe | AGGCCACGGACG | 60 |
| | | | ATCCGCAAACGCCC/3C6/ | |
| 17 | CLEC11A | Forward | GCGGGAGTTTGGCGTAG | 61 |
| | | Primer | | |
| | | Reverse | CGCGCAAATACCGAATAAACG | 62 |
| | | Primer | | |
| | | Probe | CGCGCCGAGG | 63 |
| | | | GTCGGTAGATCGTTAGTAGATG/3C6/ | |
| | | Probe | AGGCCACGGACG | 64 |
| | | | GTCGGTAGATCGTTAGTAGATG/3C6/ | |
| 8 | TSYPL5 | Forward | TTTGTTTCGGTTTTTGGCG | 65 |
| | | Primer | | |
| | | Reverse | CGCCACCATAAACGACC | 66 |
| | | Primer | | |
| | | Probe | AGGCCACGGACG | 67 |
| | | | GCGGGATTTTCGATTTC/3C6/ | |
| 18 | HOXA1 | Forward | AGTCGTTTTTTTAGGTAGTTTAGGC | 68 |
| | | Primer | G | |
| | | Reverse | CGACCTTTACAATCGCCGC | 69 |
| | | Primer | | |
| | | Probe | CGCGCCGAGG | 70 |
| | | | GGCGGTAGTTGTTGC/3C6/ | |
| 16 | EMX1 | Forward | GGCGTCGCGTTTTTTAGAGAA | 71 |
| | | Primer | | |
| | | Reverse | TTCCTTTTCGTTCGTATAAAATTTCG | 72 |
| | | Primer | T | |
| | | Probe | CGCGCCGAGG | 73 |
| | | | ATCGGGTTTTAGCGATGTT/3C6/ | |
| 6 | PPP2R5C | Forward | GCGGTAGGAGGGTTCGG | 74 |
| | | Primer | | |
| | | Reverse | GCAGGTGCCAGAACAGT | 75 |
| | | Primer | | |
| | | | CGCGCCGAGG | 76 |
| | | Probe | GCGGGAGTTTACGCG/3C6/ | |
| 10 | B3GALT6 | Forward | TGGACTGAGACTCCTGTTCTG | 77 |
| | | Primer | | |
| | | Reverse | CTCGACCTCACTCCTATTATCGC | 78 |
| | | Primer | | |
| | | Probe | ACGGACGCGGAGGTGGCCGTCTTA | 79 |
| | | | TTCAGC/3C6/ | |
| 27 | IFFO1 | Forward | CGGGATAGAGTCGATTAATTAGGC | 80 |
| | | Primer | | |
| | | Reverse | TAACTTCCCCTCGACCCG | 81 |
| | | Primer | | |
| | | Probe | CGCGCCGAGG | 82 |
| | | | CGGTTCGGTAGCGG/3C6/ | |
| 28 | HOXA9 | Forward | TTGGGTAATTATTACGTGGATTCG | 83 |
| | | Primer | | |
| | | Reverse | CAACTCATCCGCGACG | 84 |
| | | Primer | | |
| | | Probe | AGGCCACGGACG | 85 |
| | | | GTCGACGCCCAACAA/3C6/ | |
| 28 | HOXA9 | Forward | GGGTTAGGCGTTGGGTACG | 86 |
| | | Primer | | |
| | | Reverse | AACTCATCCGCGACGTCG | 87 |
| | | Primer | | |
| | | Probe | AGGCCACGGACG | 88 |
| | | | GACGCCCAACAAAAACG/3C6/ | |
| 29 | HOPX | Forward | GTAGCGCGTAGGGATTATGTCG | 89 |
| | | Primer | | |
| | | Reverse | TTTCCACCTAATCCTCTATAAAACC | 90 |
| | | Primer | GC | |
| | | Probe | AGGCCACGGACG | 91 |
| | | | CTCGCGATCTCCGC/3C6/ | |
| 30 | OSR2 | Forward | TGGAGTTATCGGAAGGCGA | 92 |
| | | Primer | | |
| | | Reverse | CGAACTCCCGAAACGACG | 93 |
| | | Primer | | |
| | | Probe | CGCGCCGAGG | 94 |
| | | | GCGCGAACACAAAACG/3C6/ | |
| 31 | QKI | Forward | GTTCGGCGTAGAGTTTCGTAGA | 95 |
| | | Primer | | |
| | | Reverse | GAAAATAAAAATTTAAAACTTTTCGA | 96 |
| | | Primer | AACGCG | |
| | | Probe | CGCGCCGAGG | 97, |
| | | | GTACCGCGACGTCC/3C6/ | 98 |
| | | | AGCTCGTCCGACA | |
| | | | GTACCGCGACGTCC/3C6/ | |
| 32 | RYR2 | Forward | GGAGGTTTCGCGTTTCGATTA | 99 |
| | | Primer | | |
| | | Reverse | CGAACGATCCCCGCCTAC | 100 |
| | | Primer | | |
| | | Probe | AGGCCACGGACG | 101 |
| | | | ATTCGCGTTCGAGCG/3C6/ | |
| 33 | GPRIN1 | Forward | TCGCGTCGTCGTTCGT | 102 |
| | | Primer | | |
| | | Reverse | GACGCCATCTAAAAACGCGA | 103 |
| | | Primer | | |
| | | Probe | CGCGCCGAGG | 104, |
| | | | TCGTTCGTGTCGGTTTC/3C6/ | 105, |
| | | | AGCTCGTCCGACA | 106, |
| | | | TCGTTCGTGTCGGTTTC/3C6/ | 107 |
| | | | ACGGACGCGGAG | |
| | | | TCGTTCGTGTCGGTTTC/3C6/ | |
| | | | AGGCCACGGACG | |
| | | | TCGTTCGTGTCGGTTTC/3C6/ | |
| 34 | ZN F569 | Forward | AGAGTTCGGCGTTTAGAGTTAGC | 108 |
| | | Primer | | |
| | | Reverse | TTAAATATAAAATCGAAACCTATATC | 109 |
| | | Primer | CGCG | |
| | | Probe | AGGCCACGGACG | 110 |
| | | | CGGTTTTTCGAGGATTTATTATTAA | |
| | | | G/3C6/ | |
| 35 | CD1D | Forward | GGAGAAGAGTGCGTAGGTTAGAG | 111 |
| | | Primer | | |
| | | Reverse | CATATCGCCCGACGTAAAAACC | 112 |
| | | Primer | | |
| | | Probe | CGCGCCGAGG | 113, |
| | | | CTCGCGAAACGCCG/3C6/ | 114, |
| | | | AGCTCGTCCGACA | 115 |
| | | | CTCGCGAAACGCCG/3C6/ | |
| | | | ACGGACGCGGAG | |
| | | | CTCGCGAAACGCCG/3C6/ | |
| 36 | NTRK3 | Forward | AGAGTTGGCGAGTTGGTTGTAC | 116 |
| | | Primer | | |
| | | Reverse | CGAATTACAACAAAACCGAATAACG | 117 |
| | | Primer | CGA | |
| | | Probe | CGCGCCGAGG | 118 |
| | | | CGATACGGAAAGGCGT/3C6/ | |
| 37 | VAV3 | Forward | TCGGAGTCGAGTTTAGCGC | 119 |
| | | Primer | | |
| | | Reverse | CGAAATCGAAAAAACAAAAACCGC | 120 |
| | | Primer | | |
| | | Probe | AGGCCACGGACG | 121, |
| | | | CGGCGTTCGCGATT/3C6/ | 122, |
| | | | CGCGCCGAGG | 123 |
| | | | CGGCGTTCGCGATT/3C6/ | |
| | | | ACGGACGCGGAG | |
| | | | CGGCGTTCGCGATT/3C6/ | |
| 38 | FAM59B | Forward | CGCGATAGCGTTTTTTATTGTCGCG | 124 |
| | | Primer | | |
| | | Reverse | CGCACGACCGTAAAATACTCG | 125 |
| | | Primer | AGGCCACGGACG | 126 |
| | | Probe | GTCGAAATCGAAACGCTC/3C6/ | |

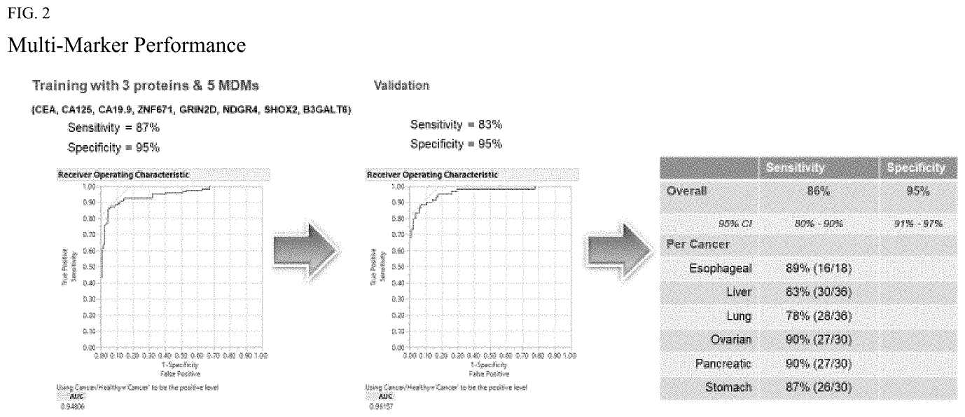

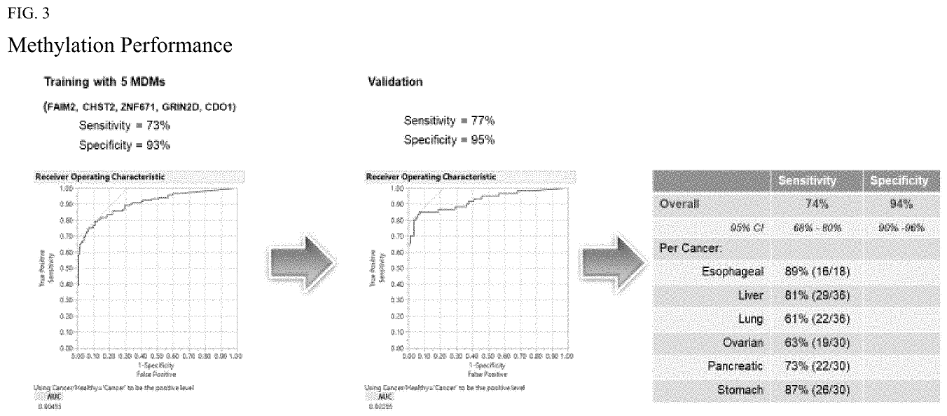

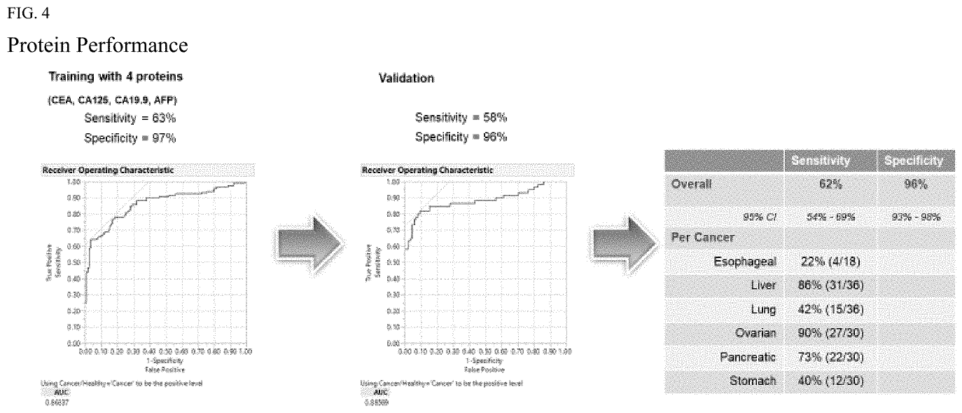

Additionally, 5 protein markers (CEA, CA125, CA19-9, AFP, CA-15-3) were tested from paired serum aliquots and combined with MDMs for a multi-analyte analysis. Two-thirds of the cases and controls were used to develop prediction algorithms, where logistic regression analysis was performed for 1) methylation markers only (FIG. 3); 2) protein markers only (FIG. 4); and 3) methylation markers and protein markers (FIG. 2), and random forest analysis was also performed. The remaining ⅓ were used to validate the models.

As shown in Table 3 and FIG. 2, for DMRs 1-26 (Table 1), using stepwise logistic regression, a combination of 3 proteins (CEA, CA125, CA19-9) and 5 MDMs (ZNF671, GRIN2D, NDGR4, SHOX2, B3GALT6) resulted in an area under the receiver operating characteristics curve (AUC) of 0.95 and an overall sensitivity of 87% for all cancers at 95% specificity. The logistic model on the validation set of the samples resulted in an AUC of 0.96 and a sensitivity of 83% with an observed specificity of 94%. The cancer-specific sensitivities ranged from 78% for lung cancer to 90% for ovarian and pancreatic cancer. Random forest and logistic analyses were highly concordant. FIG. 3 shows that a combination of 5 MDMs (FAIM2, CHST2, ZNF671, GRIN2D, CDO1) resulted in an overall sensitivity of 74% for all cancers at 94% specificity. FIG. 4 shows that a combination of 4 proteins (CEA, CA125, CA19.9, AFP) resulted in an overall sensitivity of 62% for all cancers at 96% specificity.

TABLE 3
Performance with 95% confidence intervals of analytic models of multi-
target assay of selected MDM and proteins for multi-cancer detection.

| | Sensitivity | | | | | | | |
|---|---|---|---|---|---|---|---|---|
| | Model | | | | | | | |
| | | By cancer type | | | | | | |
| Logistic | Overall | Lung | Esophageal | Gastric | Pancreatic | Liver | Ovarian | Specificity |
| Training | 87% | 79% | 83% | 90% | 95% | 88% | 85% | 95% |
| set | (79-92%) | | | | | | | (90-97%) |
| Test set | 83% | 75% | 100% | 80% | 80% | 75% | 100% | 94% |
| | (72-91%) | | | | | | | (87%-98%) |
| Training + | 86% | 78% | 89% | 87% | 90% | 83% | 90% | 95% |
| Test¹ | (80-90%) | | | | | | | (91-97%) |
| Training + | 75% | 50% | 89% | 83% | 76% | 81% | 80% | 98% |
| Test² | (68-81%) | | | | | | | (96-99%) |
| Random | 77% | 58% | 78% | 83% | 77% | 81% | 87% | 95% |
| forest³ | (70-83%) | | | | | | | (92-97%) |

¹95% specificity goal, 5 MDMs and 3 proteins
²98% specificity goal, 5 MDMs and 3 proteins
³With 500-fold in silico cross-validation on all samples and all markers (26 MDMs + 5 proteins)

Similar experiments were conducted with collected plasmas from 160 cases of six cancer types (esophageal (n=15), hepatocellular carcinoma (HCC)/liver (n=29), lung (n=29), ovarian (n=29), pancreatic (n=30), and stomach (n=28)), and 317 age and gender matched asymptomatic controls were tested in blinded fashion (see, Table 4 for overall stage of type of cancer for each subject). Testing was performed in blinded fashion using multiplex PCR followed by LQAS (Long probe Quantitative Amplified Signal) assay on bisulfite converted DNA extracted from 3 mL of plasma collected in LBgard blood tubes. Protein testing was performed on paired serum aliquots and combined with MDMs for a multi-analyte analysis. The subjects were divided into training and a validation set with equal representation of cancer type, staging, gender, and age between them. Two-thirds of the cases and controls were used to train with a logistic prediction algorithm, and the remaining ⅓ were used to validate the model.

As shown in Table 5, using stepwise logistic regression, a combination of 16 MDMs (GRIN2D, SHOX2, ZNF671, SIM2, TRH, CAPN2, CHST2_7890, FER1L4, FAIM2, PPP2R5C, TSPYL5, NDRG4, ZNF781, IFFO1, HOXA9, HOPX) resulted in an overall sensitivity of 87% for all cancers at 97.5% specificity. The cancer-specific sensitivities ranged from 72% for ovarian cancer to 93% for lung cancer (see, Table 5). As shown in Table 6, using stepwise logistic regression, a combination of 5 proteins (CEA, CA125, CA19-9, AFP, CA-15-3) resulted in an overall sensitivity of 84% for all cancers at 98% specificity. The cancer-specific sensitivities ranged from 80% for esophageal cancer to 86% for liver cancer, ovarian cancer, pancreatic cancer, and stomach cancer (see, Table 6). As shown in Table 7, a combination of 16 MDMs (GRIN2D, SHOX2, ZNF671, SIM2, TRH, CAPN2, CHST2_7890, FER1L4, FAIM2, PPP2R5C, TSPYL5, NDRG4, ZNF781, IFFO1, HOXA9, HOPX) and 5 proteins (CEA, CA125, CA19-9, AFP, CA-15-3) resulted in an overall sensitivity of 85% for all cancers at 98% specificity. The cancer-specific sensitivities ranged from 80% for esophageal cancer to 86% for liver cancer, lung cancer, ovarian cancer, and stomach cancer (see, Table 7).

TABLE 4

| | Overall Stage | | | | |
|---|---|---|---|---|---|
| Cancer Type | I | II | III | IV | NA |
| Esophageal | 0 | 1 | 6 | 8 | 0 |
| HCC | 8 | 6 | 10 | 5 | 0 |
| Lung | 2 | 3 | 12 | 4 | 8 |
| Ovarian | 2 | 1 | 9 | 6 | 11 |
| Pancreatic | 0 | 12 | 5 | 13 | 0 |
| Stomach | 4 | 6 | 5 | 13 | 0 |

TABLE 5

| | Sensitivity | Specificity |
|---|---|---|
| Overall | 79% | 97.5% |
| Per Cancer | | |
| Esophageal | 73% (11/15) | |
| Liver | 79% (23/29) | |
| Lung | 93% (27/29) | |
| Ovarian | 72% (21/29) | |
| Pancreatic | 67% (20/30) | |
| Stomach | 86% (24/28) | |

TABLE 6

| | Sensitivity | Specificity |
|---|---|---|
| Overall | 84% | 98% |
| Per Cancer | | |
| Esophageal | 80% (12/15) | |
| Liver | 86% (25/29) | |
| Lung | 79% (15/19) | |
| Ovarian | 86% (24/28) | |
| Pancreatic | 86% (25/29) | |
| Stomach | 86% (24/28) | |

TABLE 7

| | Sensitivity | Specificity |
|---|---|---|
| Overall | 85% | 98% |
| Per Cancer | | |
| Esophageal | 80% (12/15) | |
| Liver | 86% (25/29) | |
| Lung | 86% (25/29) | |
| Ovarian | 86% (25/29) | |
| Pancreatic | 83% (25/30) | |
| Stomach | 86% (24/28) | |

Similar additional experiments were conducted with collected plasmas from 236 cases of 13 cancer types (esophageal (n=1), bladder (n=20), breast cancer (n=14), cervical cancer (n=10), colorectal cancer (n=38), hepatocellular carcinoma (HCC)/liver (n=13), lung (n=40), ovarian (n=8), pancreatic (n=30), prostate cancer (n=10), renal cancer (n=20), stomach cancer (n=22), and uterine cancer (n=10)), and 146 age and gender matched asymptomatic controls were tested in blinded fashion (see, Table 8 for overall stage of type of cancer for each subject). Table 9 shows the percentage methylation cutoff and percentage sensitivity for all 13 cancer types for the following MDMs: GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B. Tables 10, 11 and 12 shows the overall sensitivity for the 13 cancer types for the following DMRs: TRH, EMX1, and FAIM2, respectively.

As shown in Table 13, a combination of the thirty-five MDMs (GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B) within a triplex QuARTS assay resulted in overall sensitivity of 74% for all cancers (bladder, breast, cervical, CRC, esophageal, HCC, lung, ovarian, pancreatic, prostate, renal, stomach, uterine) at 97% specificity. The cancer-specific sensitivities ranged from 20% for pancreatic to 100% for cervical, esophageal, ovarian, and uterine.

As further shown in Table 13, a combination of the thirty-five MDMs (GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B) and a combination of 5 proteins (CEA, CA125, CA19-9, AFP, CA-15-3) within a triplex QuARTS assay resulted in overall sensitivity of 78% for all cancers (bladder, breast, cervical, CRC, esophageal, HCC, lung, ovarian, pancreatic, prostate, renal, stomach, uterine) at 97% specificity. The cancer-specific sensitivities ranged from 20% for pancreatic to 100% for cervical, esophageal, ovarian, and uterine.

As further shown in Table 13, a combination of the thirty-five MDMs (GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B) within a multi-analyte to three dyes reaction (MAD) LQAS assay resulted in overall sensitivity of 72% for all cancers (bladder, breast, cervical, CRC, esophageal, HCC, lung, ovarian, pancreatic, prostate, renal, stomach, uterine) at 97% specificity. The cancer-specific sensitivities ranged from 20% for pancreatic to 100% for cervical and esophageal.

As further shown in Table 13, a combination of the thirty-five MDMs (GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B) and a combination of 5 proteins (CEA, CA125, CA19-9, AFP, CA-15-3) within a MAD-LQAS assay resulted in overall sensitivity of 75% for all cancers (bladder, breast, cervical, CRC, esophageal, HCC, lung, ovarian, pancreatic, prostate, renal, stomach, uterine) at 97% specificity. The cancer-specific sensitivities ranged from 20% for pancreatic to 100% for cervical, esophageal, ovarian, and uterine.

Additional experiments were conducted performance for bladder cancer, breast cancer, cervical cancer, CRC, esophageal cancer, HCC, lung cancer, ovarian cancer, pancreatic cancer, renal cancer, stomach cancer, and uterine cancer, but excluding prostate cancer (see, Table 14).

As shown in Table 14, a combination of the thirty-five MDMs (GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B) within a triplex QuARTS assay resulted in overall sensitivity of 77% for all cancers (bladder, breast, cervical, CRC, esophageal, HCC, lung, ovarian, pancreatic, renal, stomach, uterine) (not including prostate cancer) at 97% specificity. The cancer-specific sensitivities ranged from 50% for renal to 100% for cervical, esophageal, ovarian, and uterine.

As further shown in Table 14, a combination of the thirty-five MDMs (GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B) and a combination of 5 proteins (CEA, CA125, CA19-9, AFP, CA-15-3) within a triplex QuARTS assay resulted in overall sensitivity of 81% for all cancers (bladder, breast, cervical, CRC, esophageal, HCC, lung, ovarian, pancreatic, renal, stomach, uterine) (not including prostate cancer) at 97% specificity. The cancer-specific sensitivities ranged from 50% for renal to 100% for cervical, esophageal, ovarian, and uterine.

As further shown in Table 14, a combination of the thirty-five MDMs (GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B) within a MAD-LQAS assay resulted in overall sensitivity of 74% for all cancers (bladder, breast, cervical, CRC, esophageal, HCC, lung, ovarian, pancreatic, renal, stomach, uterine) (not including prostate cancer) at 97% specificity. The cancer-specific sensitivities ranged from 50% for renal to 100% for cervical and esophageal.

As further shown in Table 14, a combination of the thirty-five MDMs (GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B) and a combination of 5 proteins (CEA, CA125, CA19-9, AFP, CA-15-3) within a MAD-LQAS assay resulted in overall sensitivity of 78% for all cancers (bladder, breast, cervical, CRC, esophageal, HCC, lung, ovarian, pancreatic, renal, stomach, uterine) (not including prostate cancer) at 97% specificity. The cancer-specific sensitivities ranged from 50% for renal to 100% for cervical, esophageal, and ovarian.

The experiments additionally resulted in identification of the following panel of markers for identifying multiple types of cancer from a biological sample: CDO1, GRIN2D, SHOX2, OSR2, QKI, SIM2, TRH, CAPN2, SFMBT2, CHST2, ST8SIA1, HOXA1, FER1L4, FAIM2, IFFO1, EMX1, ZNF671, PRKCB, HOXB2, BARX1, PPP2R5C, and TSPYL5.

TABLE 8

| | | Stage | | | | |
|---|---|---|---|---|---|---|
| Cancer Type | Total | I | II | III | IV | N/A |
| Bladder | 20 | | 5 | 8 | 5 | 2 |
| Breast | 14 | | 6 | 4 | 4 | |
| Cervical | 10 | | 4 | 3 | 3 | |
| CRC | 38 | | 14 | 11 | 6 | 7 |
| Esophageal | 1 | | | | | 1 |
| HCC | 13 | 3 | 1 | 3 | 5 | 1 |
| Lung | 40 | | 7 | 6 | 13 | 14 |
| Ovarian | 8 | | | | | 8 |
| Pancreatic | 30 | | 12 | | 13 | 5 |
| Prostate | 10 | | 3 | 4 | 3 | |
| Renal | 20 | | 1 | 8 | 8 | 3 |
| Stomach | 22 | | 6 | 9 | 7 | |
| Uterine | 10 | | 4 | 3 | 3 | |

TABLE 9

| | % Methylation | % Sensitivity |
|---|---|---|
| MDM | Cutoff | All Cancers |
| EMX1 | 0.3% | 43% |
| CDO1 | 0.7% | 43% |
| FAIM2 | 0.2% | 41% |
| PRKCB | 0.1% | 38% |
| TRH | 0.9% | 37% |
| SIM2 | 0.6% | 37% |
| GRIN2D | 0.3% | 36% |
| SHOX2 | 2.8% | 34% |
| RYR2 | 0.3% | 33% |
| OSR2 | 0.6% | 32% |
| HOXA9 | 0.9% | 32% |
| IFFO1 | 0.3% | 31% |
| ZNF671 | 0.4% | 31% |
| BARX1 | 0.6% | 31% |
| ZNF781 | 1.1% | 31% |
| CAPN2 | 0.2% | 30% |
| SHISA9 | 0.4% | 30% |
| FER1L4 | 0.3% | 26% |
| PPP2R5C | 0.5% | 26% |
| ST8SIA1 | 0.1% | 25% |
| HOXA1 | 0.3% | 25% |
| NDRG4 | 0.1% | 25% |
| CHST2 | 0.0% | 25% |
| VAV3 | 0.3% | 25% |
| SFMBT2 | 0.0% | 25% |
| HOXB2 | 0.2% | 25% |
| NTRK3 | 0.6% | 24% |
| CLEC11A | 0.3% | 22% |
| GPRIN1 | 2.6% | 21% |
| TSPYL5 | 4.7% | 19% |
| FAM59B | 0.7% | 18% |
| ZNF569 | 0.3% | 18% |
| ARHGEF4 | 1.5% | 14% |
| QKI | 0.2% | 14% |
| CD1D | 5.1% | 14% |

TABLE 10
TRH

| | |
|---|---|
| Overall | 37% |
| Sensitivity | |
| Bladder | 30% |
| Breast | 50% |
| Cervical | 60% |
| CRC | 32% |
| Esophageal | 0% |
| HCC | 31% |
| Lung | 45% |
| Ovarian | 50% |
| Pancreatic | 30% |
| Prostate | 10% |
| Renal | 15% |
| Stomach | 55% |
| Uterine | 60% |
| Overall | 100% |
| Specificity | |

TABLE 11
EMX1

| | |
|---|---|
| Overall | 44% |
| Sensitivity | |
| Bladder | 35% |
| Breast | 50% |
| Cervical | 80% |
| CRC | 37% |
| Esophageal | 100% |
| HCC | 46% |
| Lung | 48% |
| Ovarian | 25% |
| Pancreatic | 47% |
| Prostate | 10% |
| Renal | 30% |
| Stomach | 64% |
| Uterine | 40% |
| Overall | 100% |
| Specificity | |

TABLE 12
FAIM2

| | |
|---|---|
| Overall | 42% |
| Sensitivity | |
| Bladder | 20% |
| Breast | 43% |
| Cervical | 60% |
| CRC | 45% |
| Esophageal | 100% |
| HCC | 31% |
| Lung | 35% |
| Ovarian | 50% |
| Pancreatic | 40% |
| Prostate | 10% |
| Renal | 20% |
| Stomach | 77% |
| Uterine | 90% |
| Overall | 100% |
| Specificity | |

TABLE 13

| MDM | | MDM + Protein | | MAD | | MAD + Protein | |
|---|---|---|---|---|---|---|---|
| Overall | 74% | Overall | 78% | Overall | 72% | Overall | 75% |
| Sensitivity | | Sensitivity | | Sensitivity | | Sensitivity | |
| Overall | 97% | Overall | 97% | Overall | 97% | Overall | 97% |
| Specificity | | Specificity | | Specificity | | Specificity | |
| Bladder | 55%(11/20) | Bladder | 55%(11/20) | Bladder | 55%(11/20) | Bladder | 55%(11/20) |
| Breast | 71%(10/14) | Breast | 71%(10/14) | Breast | 64%(9/14) −1 | Breast | 64%(9/14) |
| Cervical | 100%(10/10) | Cervical | 100%(10/10) | Cervical | 100%(10/10) | Cervical | 100%(10/10) |
| CRC | 74%(28/38) | CRC | 79%(30/38) | CRC | 71%(27/38) −1 | CRC | 74%(28/38) |
| Esophageal | 100%(1/1) | Esophageal | 100%(1/1) | Esophageal | 100%(1/1) | Esophageal | 100%(1/1) |
| HCC | 62%(8/13) | HCC | 77%(10/13) | HCC | 69%(9/13) +1 | HCC | 85%(11/13) |
| Lung | 88%(35/40) | Lung | 93%(37/40) | Lung | 85%(34/40) −1 | Lung | 90%(36/40) |
| Ovarian | 100%(8/8) | Ovarian | 100%(8/8) | Ovarian | 88%(7/8) −1 | Ovarian | 100%(8/8) |
| Pancreatic | 73%(22/30) | Pancreatic | 83%(25/30) | Pancreatic | 73%(22/30) | Pancreatic | 80%(24/30) |
| Prostate | 20%(2/10) | Prostate | 20%(2/10) | Prostate | 20%(2/10) | Prostate | 20%(2/10) |
| Renal | 50%(10/20) | Renal | 50%(10/20) | Renal | 50%(10/20) | Renal | 50%(10/20) |
| Stomach | 91%(20/22) | Stomach | 91%(20/22) | Stomach | 88%(19/22) −1 | Stomach | 86%(19/22) |
| Uterine | 100%(10/10) | Uterine | 100%(10/10) | Uterine | 90%(9/10) −1 | Uterine | 90%(9/10) |
| All Cancers | 74%(175/236) | All Cancers | 78%(184/236) | All Cancers | 72%(170/236) −5 | All Cancers | 75%(178/236) |

*Proteins used: AFP, CEA, CA125. CA15-3, CYFRA-1, CA-19.9
*Individal and ratios of free and total PSA did not add sensitivity

TABLE 14

| MDM | | MDM + Protein | | MAD | | MAD + Protein | |
|---|---|---|---|---|---|---|---|
| Overall | 77% | Overall | 81% | Overall | 74% | Overall | 78% |
| Sensitivity | | Sensitivity | | Sensitivity | | Sensitivity | |
| Overall | 97% | Overall | 97% | Overall | 97% | Overall | 97% |
| Specificity | | Specificity | | Specificity | | Specificity | |
| Bladder | 55%(11/20) | Bladder | 55%(11/20) | Bladder | 55%(11/20) | Bladder | 55%(11/20) |
| Breast | 71%(10/14) | Breast | 71%(10/14) | Breast | 64%(9/14) −1 | Breast | 64%(9/14) |
| Cervical | 100%(10/10) | Cervical | 100%(10/10) | Cervical | 100%(10/10) | Cervical | 100%(10/10) |
| CRC | 74%(28/38) | CRC | 79%(30/38) | CRC | 71%(27/38) −1 | CRC | 74%(28/38) |
| Esophageal | 100%(1/1) | Esophageal | 100%(1/1) | Esophageal | 100%(1/1) | Esophageal | 100%(1/1) |
| HCC | 62%(8/13) | HCC | 77%(10/13) | HCC | 69%(9/13) +1 | HCC | 85%(11/13) |
| Lung | 88%(35/40) | Lung | 93%(37/40) | Lung | 85%(34/40) −1 | Lung | 90%(36/40) |
| Ovarian | 100%(8/8) | Ovarian | 100%(8/8) | Ovarian | 88%(7/8) −1 | Ovarian | 100%(8/8) |
| Pancreatic | 73%(22/30) | Pancreatic | 83%(25/30) | Pancreatic | 73%(22/30) | Pancreatic | 80%(24/30) |
| Renal | 50%(10/20) | Renal | 50%(10/20) | Renal | 50%(10/20) | Renal | 50%(10/20) |
| Stomach | 91%(20/22) | Stomach | 91%(20/22) | Stomach | 86%(19/22) −1 | Stomach | 86%(19/22) |
| Uterine | 100%(10/10) | Uterine | 100%(10/10) | Uterine | 90%(9/10) −1 | Uterine | 90%(9/10) |
| All Cancers | 77%(173/226) | All Cancers | 81%(181/226) | All Cancers | 74%(168/226) −5 | All Cancers | 78%(176/226) |

All publications and patents mentioned in the above specification are herein incorporated by reference in their entirety for all purposes. Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

1. A method for characterizing a biological sample comprising one or both of:
a) measuring a methylation level of one or more methylated markers selected from FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, ST8SIA1, IFFO1, HOXA9, HOPX, OSR2, QKI, RYR2, GPRIN1, ZNF569, CD1D, NTRK3, VAV3, and FAM59B in the biological sample, wherein measuring a methylation level of one or more methylated markers comprises treating DNA from the biological sample with a reagent that modifies DNA in a methylation-specific manner; and
b) measuring an expression and/or activity level of one or more protein markers selected from CEA, CA125, CA19.9, AFP, and CA-15-3 in the biological sample.

2. The method of claim 1,
wherein if a methylation level of one or more methylated markers is measured, then the measured methylation level of the one or more methylation markers is compared to a methylation level of a corresponding one or more methylation markers in control samples without a specific type of cancer; and/or
wherein if an expression and/or activity level of one or more protein markers is measured, then the measured expression and/or activity level of the one or more protein markers is compared to an expression and/or activity level of a corresponding one or more protein markers in control samples without a specific type of cancer.

3. The method of claim 2, further comprising determining that the individual has one or more types of cancer when one or both of:
i) the methylation level measured in the one or more methylation markers is higher than the methylation level measured in the respective control samples; and
ii) the expression and/or activity level of one or more protein markers is higher than the expression and/or activity level measured in the respective control samples.

4. (canceled)

5. The method of claim 3, wherein more than one type of cancer is selected from liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer.

6. The method of claim 1, wherein the reagent that modifies DNA in a methylation-specific manner is selected from one or more of a borane reducing agent, a methylation-sensitive restriction enzyme, a methylation-dependent restriction enzyme, and a bisulfite reagent.

7. (canceled)

8. (canceled)

9. (canceled)

10. The method of claim 1, wherein the treated DNA is amplified with a set of primers specific for the one or more methylated markers.

11. The method of claim 10,
wherein the set of primers specific for the one or more methylated markers is selected from the group recited in Table 2, or
wherein the set of primers specific for the one or more methylated markers is capable of binding an amplicon bound by a primer sequence for the specific methylated marker gene recited in Table 2, wherein the amplicon bound by the primer sequence for the methylated marker gene recited in Table 2 is at least a portion of a genetic region for the methylated marker recited in Table 1, or
wherein the set of primers specific for the one or more methylated markers is a set of primers that specifically binds at least a portion of a genetic region comprising chromosomal coordinates for a methylated marker recited in Table 1.

12. (canceled)

13. (canceled)

14. (canceled)

15. The method of claim 1, wherein measuring a methylation level of one or more methylated markers comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

16. The method of claim 1, wherein measuring a methylation level of one or more methylated markers comprises measuring methylation of a CpG site for the one or more methylation markers.

17. (canceled)

18. The method of claim 1, wherein the one or more methylated markers is described by the genomic coordinates shown in Table 1.

19. The method of claim 1, wherein the biological sample is a stool sample, a tissue sample, an organ secretion sample, a CSF sample, a saliva sample, a blood sample, a plasma sample, or a urine sample,
wherein the biological sample is from a human subject having or suspected of having cancer.

20. (canceled)

21. (canceled)

22. The method of claim 1, wherein the one or more methylated markers are selected from one of the following groups:
FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, and ST8SIA1;
GRIN2D, SHOX2, ZNF671, SIM2, TRH, CAPN2, CHST2_7890, FER1L4, FAIM2, PPP2R5C, TSPYL5, NDRG4, ZNF781, IFFO1, HOXA9, and HOPX;
GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B;
CDO1, GRIN2D, SHOX2, OSR2, QKI, SIM2, TRH, CAPN2, SFMBT2, CHST2, ST8SIA1, HOXA1, FER1L4, FAIM2, IFFO1, EMX1, ZNF671, PRKCB, HOXB2, BARX1, PPP2R5C, and TSPYL5;
ZNF671, GRIN2D, NDGR4, SHOX2, B3GALT6; and
FAIM2, CHST2, ZNF671, GRIN2D, CDO1.

23. A method for preparing a deoxyribonucleic acid (DNA) fraction from a biological sample useful for analyzing one or more genetic loci involved in one or more chromosomal aberrations, comprising:
(a) extracting genomic DNA from a biological sample;
(b) producing a fraction of the extracted genomic DNA by:
(i) treating the extracted genomic DNA with a reagent that modifies DNA in a methylation-specific manner;
(ii) amplifying the treated genomic DNA using separate primers specific for one or more of the following methylation markers: FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, ST8SIA1, IFFO1, HOXA9, HOPX, OSR2, QKI, RYR2, GPRIN1, ZNF569, CD1D, NTRK3, VAV3, and FAM59B;
(c) analyzing one or more genetic loci in the produced fraction of the extracted genomic DNA by measuring a methylation level for each of the one or more methylation markers.

24. The method of claim 23, wherein the reagent that modifies DNA in a methylation-specific manner is selected from one or more of a borane reducing agent, a methylation-sensitive restriction enzyme, a methylation-dependent restriction enzyme, and a bisulfite reagent.

25. (canceled)

26. (canceled)

27. (canceled)

28. The method of claim 23,
wherein the set of primers specific for the one or more methylated markers is selected from the group recited in Table 2, or
wherein the set of primers specific for the one or more methylated markers is capable of binding an amplicon bound by a primer sequence for the specific methylated marker gene recited in Table 2, wherein the amplicon bound by the primer sequence for the methylated marker gene recited in Table 2 is at least a portion of a genetic region for the methylated marker recited in Table 1, or
wherein the set of primers specific for the one or more methylated markers is a set of primers that specifically binds at least a portion of a genetic region comprising chromosomal coordinates for a methylated marker recited in Table 1.

29. (canceled)

30. (canceled)

31. (canceled)

32. The method of claim 23, wherein measuring a methylation level of one or more methylated markers comprises using one or more methods selected from the group consisting of methylation-specific PCR, quantitative methylation-specific PCR, methylation-specific DNA restriction enzyme analysis, quantitative bisulfite pyrosequencing, flap endonuclease assay, PCR-flap assay, and bisulfite genomic sequencing PCR.

33. The method of claim 23, wherein measuring a methylation level of one or more methylated markers comprises measuring methylation of a CpG site for the one or more methylation markers.

34. (canceled)

35. The method of claim 23, wherein the one or more methylated markers is described by the genomic coordinates shown in Table 1.

36. The method of claim 23, wherein the biological sample is a stool sample, a tissue sample, an organ secretion sample, a CSF sample, a saliva sample, a blood sample, a plasma sample, or a urine sample,
wherein the biological sample is from a human subject having or suspected of having cancer.

37. (canceled)

38. (canceled)

39. The method of claim 23, wherein the one or more methylated markers are selected from one of the following groups:
FAIM2, CDO1, SIM2, CHST_7890, SFMBT2, PPP2R5C, ARHGEF4, TSPYL5, ZNF671, B3GALT6, FER1L4, HOXB2, BARX1, TBX1, SHOX2, EMX1, CLEC11A, HOXA1, GRIN2D, CAPN2, NDRG4, TRH, PRKCB, SHISA9, ZNF781, and ST8SIA1;
GRIN2D, SHOX2, ZNF671, SIM2, TRH, CAPN2, CHST2_7890, FER1L4, FAIM2, PPP2R5C, TSPYL5, NDRG4, ZNF781, IFFO1, HOXA9, and HOPX;
GRIN2D, SHOX2, ZNF671, SIM2, TRH, CAPN2, CHST2_7890, FER1L4, FAIM2, PPP2R5C, TSPYL5, NDRG4, ZNF781, CDO1, EMX1, PRKCB, SFMBT2, ST8SIA1, HOXA1, HOXB2, BARX1, CLEC11A, ARHGEF4, IFFO1, HOXA9, OSR2, QKI, RYR2, GPRIN1, ZNF569, SHISA9, CD1D, NTRK3, VAV3, and FAM59B;
CDO1, GRIN2D, SHOX2, OSR2, QKI, SIM2, TRH, CAPN2, SFMBT2, CHST2, ST8SIA1, HOXA1, FER1L4, FAIM2, IFFO1, EMX1, ZNF671, PRKCB, HOXB2, BARX1, PPP2R5C, and TSPYL5;
ZNF671, GRIN2D, NDGR4, SHOX2, B3GALT6; and
FAIM2, CHST2, ZNF671, GRIN2D, CDO1.

40. (canceled)

41. (canceled)

42. The method of claim 23, wherein each of the analyzed one or more genetic loci is associated with one or more of liver cancer, esophageal cancer, lung cancer, ovarian cancer, pancreatic cancer, gastric cancer, bladder cancer, breast cancer, cervical cancer, colorectal cancer, prostate cancer, renal cancer, and uterine cancer.


SEQUENCE LISTING (236 sequences)

SEQ ID NO: 1 - DNA, 21 bp, Artificial Sequence
  synthetic
cgaaacgtaaggatgtcgtcg

SEQ ID NO: 2 - DNA, 27 bp, Artificial Sequence
  synthetic
aatttatatatacaccgcgtctccaac

SEQ ID NO: 3 - DNA, 28 bp, Artificial Sequence
  synthetic
cgcgccgaggcgatcccgaatccactac

SEQ ID NO: 4 - DNA, 16 bp, Artificial Sequence
  synthetic
ttgcggaggacgttgc

SEQ ID NO: 5 - DNA, 21 bp, Artificial Sequence
  synthetic
gaaaaaaaacgatacgccgcc

SEQ ID NO: 6 - DNA, 26 bp, Artificial Sequence
  synthetic
cgcgccgaggcggattcgcgagttcg

SEQ ID NO: 7 - DNA, 20 bp, Artificial Sequence
  synthetic
gcgcggaattttaggagtgc

SEQ ID NO: 8 - DNA, 18 bp, Artificial Sequence
  synthetic
cgcgaccccacgataatc

SEQ ID NO: 9 - DNA, 30 bp, Artificial Sequence
  synthetic
aggccacggacgcggggttcgagtgtaaat

SEQ ID NO: 10 - DNA, 18 bp, Artificial Sequence
  synthetic
aaagggagttttcgggcg

SEQ ID NO: 11 - DNA, 19 bp, Artificial Sequence
  synthetic
acccgatacccccattacc

SEQ ID NO: 12 - DNA, 32 bp, Artificial Sequence
  synthetic
cgcgccgaggcgtacgcaaacctaaaaaattc

SEQ ID NO: 13 - DNA, 21 bp, Artificial Sequence
  synthetic
cggttttcgttcgttttttcg

SEQ ID NO: 14 - DNA, 21 bp, Artificial Sequence
  synthetic
cgtaacttccgccttctacgc

SEQ ID NO: 15 - DNA, 35 bp, Artificial Sequence
  synthetic
aggccacggacggttcgtttatcgggtattttagt

SEQ ID NO: 16 - DNA, 19 bp, Artificial Sequence
  synthetic
ggttcgggagaaggttcgg

SEQ ID NO: 17 - DNA, 26 bp, Artificial Sequence
  synthetic
cgaaaaacgacgaaaaacgcaaaaac

SEQ ID NO: 18 - DNA, 35 bp, Artificial Sequence
  synthetic
cgcgccgaggcatcgctcgaaaaaaacaaaaaaac

SEQ ID NO: 19 - DNA, 23 bp, Artificial Sequence
  synthetic
cgttcgcgttatttatttcggcg

SEQ ID NO: 20 - DNA, 24 bp, Artificial Sequence
  synthetic
gctcctaattctcatcaacgtcgt

SEQ ID NO: 21 - DNA, 24 bp, Artificial Sequence
  synthetic
cgcgccgagggcggcgttttgcgc

SEQ ID NO: 22 - DNA, 24 bp, Artificial Sequence
  synthetic
tgttatgggttagtgggattcgtc

SEQ ID NO: 23 - DNA, 20 bp, Artificial Sequence
  synthetic
ccgaaaaccacaaatcccgc

SEQ ID NO: 24 - DNA, 30 bp, Artificial Sequence
  synthetic
cgcgccgaggcgtttaattgtagttcgggc

SEQ ID NO: 25 - DNA, 24 bp, Artificial Sequence
  synthetic
gttgttttatatatcggcgttcgg

SEQ ID NO: 26 - DNA, 24 bp, Artificial Sequence
  synthetic
actacgactatacacgcttaaccg

SEQ ID NO: 27 - DNA, 27 bp, Artificial Sequence
  synthetic
cgcgccgaggggttatcgcgggtttcg

SEQ ID NO: 28 - DNA, 19 bp, Artificial Sequence
  synthetic
gtcgtcgttcgagagggta

SEQ ID NO: 29 - DNA, 23 bp, Artificial Sequence
  synthetic
gaacaaaaacgaacgaacgaaca

SEQ ID NO: 30 - DNA, 28 bp, Artificial Sequence
  synthetic
cgcgccgaggatcggtttcgttcgtttg

SEQ ID NO: 31 - DNA, 18 bp, Artificial Sequence
  synthetic
gttgtcgggagcggtagg

SEQ ID NO: 32 - DNA, 21 bp, Artificial Sequence
  synthetic
ccaatatcccgaaacgcgtct

SEQ ID NO: 33 - DNA, 24 bp, Artificial Sequence
  synthetic
cgcgccgagggcgtttcgatcggg

SEQ ID NO: 34 - DNA, 19 bp, Artificial Sequence
  synthetic
cgttgacgcgtagttttcg

SEQ ID NO: 35 - DNA, 18 bp, Artificial Sequence
  synthetic
gtcgaccaaaaacgcgtc

SEQ ID NO: 36 - DNA, 26 bp, Artificial Sequence
  synthetic
cgcgccgaggcgtcccgcaactacaa

SEQ ID NO: 37 - DNA, 27 bp, Artificial Sequence
  synthetic
tcgattatgtcgttttagacgttatcg

SEQ ID NO: 38 - DNA, 28 bp, Artificial Sequence
  synthetic
tctacatcgacattctaaaacgactaac

SEQ ID NO: 39 - DNA, 30 bp, Artificial Sequence
  synthetic
aggccacggacgcgcataccatcgacttca

SEQ ID NO: 40 - DNA, 21 bp, Artificial Sequence
  synthetic
gtatagcgcgatttcgtagcg

SEQ ID NO: 41 - DNA, 21 bp, Artificial Sequence
  synthetic
aattacctacgctatccgccc

SEQ ID NO: 42 - DNA, 31 bp, Artificial Sequence
  synthetic
aggccacggacgcgaacatcctcccgataat

SEQ ID NO: 43 - DNA, 24 bp, Artificial Sequence
  synthetic
cgttaatttgttagatagagggcg

SEQ ID NO: 44 - DNA, 17 bp, Artificial Sequence
  synthetic
tccgaacaaccgcctac

SEQ ID NO: 45 - DNA, 27 bp, Artificial Sequence
  synthetic
aggccacggacgcgaaaaatcccacgc

SEQ ID NO: 46 - DNA, 22 bp, Artificial Sequence
  synthetic
gttagaagacgttttttcgggg

SEQ ID NO: 47 - DNA, 20 bp, Artificial Sequence
  synthetic
aaaacaaaaatcgaccgcga

SEQ ID NO: 48 - DNA, 37 bp, Artificial Sequence
  synthetic
cgcgccgagggcgttaggatttatttttttttttcga

SEQ ID NO: 49 - DNA, 26 bp, Artificial Sequence
  synthetic
ttttcgttgattttattcgagtcgtc

SEQ ID NO: 50 - DNA, 22 bp, Artificial Sequence
  synthetic
gaaccctcttcaaataaaccgc

SEQ ID NO: 51 - DNA, 30 bp, Artificial Sequence
  synthetic
cgcgccgaggcgtttggcgtagatataagc

SEQ ID NO: 52 - DNA, 23 bp, Artificial Sequence
  synthetic
cgtttttttgtttttcgagtgcg

SEQ ID NO: 53 - DNA, 23 bp, Artificial Sequence
  synthetic
tcaataactaaactcaccgcgtc

SEQ ID NO: 54 - DNA, 33 bp, Artificial Sequence
  synthetic
aggccacggacggcggatttatcgggttatagt

SEQ ID NO: 55 - DNA, 23 bp, Artificial Sequence
  synthetic
tgcgtggttacggttattattcg

SEQ ID NO: 56 - DNA, 18 bp, Artificial Sequence
  synthetic
cgaccgcgacgactaaac

SEQ ID NO: 57 - DNA, 41 bp, Artificial Sequence
  synthetic
cgcgccgagggtacgcgtattcgtattattattattatttc

SEQ ID NO: 58 - DNA, 20 bp, Artificial Sequence
  synthetic
gttcgagtttaggggtagcg

SEQ ID NO: 59 - DNA, 17 bp, Artificial Sequence
  synthetic
ccgcacaaaaaaccgca

SEQ ID NO: 60 - DNA, 26 bp, Artificial Sequence
  synthetic
aggccacggacgatccgcaaacgccc

SEQ ID NO: 61 - DNA, 17 bp, Artificial Sequence
  synthetic
gcgggagtttggcgtag

SEQ ID NO: 62 - DNA, 21 bp, Artificial Sequence
  synthetic
cgcgcaaataccgaataaacg

SEQ ID NO: 63 - DNA, 32 bp, Artificial Sequence
  synthetic
cgcgccgagggtcggtagatcgttagtagatg

SEQ ID NO: 64 - DNA, 34 bp, Artificial Sequence
  synthetic
aggccacggacggtcggtagatcgttagtagatg

SEQ ID NO: 65 - DNA, 19 bp, Artificial Sequence
  synthetic
tttgtttcggtttttggcg

SEQ ID NO: 66 - DNA, 17 bp, Artificial Sequence
  synthetic
cgccaccataaacgacc

SEQ ID NO: 67 - DNA, 29 bp, Artificial Sequence
  synthetic
aggccacggacggcgggattttcgatttc

SEQ ID NO: 68 - DNA, 26 bp, Artificial Sequence
  synthetic
agtcgtttttttaggtagtttaggcg

SEQ ID NO: 69 - DNA, 19 bp, Artificial Sequence
  synthetic
cgacctttacaatcgccgc

SEQ ID NO: 70 - DNA, 25 bp, Artificial Sequence
  synthetic
cgcgccgaggggcggtagttgttgc

SEQ ID NO: 71 - DNA, 21 bp, Artificial Sequence
  synthetic
ggcgtcgcgttttttagagaa

SEQ ID NO: 72 - DNA, 27 bp, Artificial Sequence
  synthetic
ttccttttcgttcgtataaaatttcgt

SEQ ID NO: 73 - DNA, 29 bp, Artificial Sequence
  synthetic
cgcgccgaggatcgggttttagcgatgtt

SEQ ID NO: 74 - DNA, 17 bp, Artificial Sequence
  synthetic
gcggtaggagggttcgg

SEQ ID NO: 75 - DNA, 17 bp, Artificial Sequence
  synthetic
gcaggtgccagaacagt

SEQ ID NO: 76 - DNA, 25 bp, Artificial Sequence
  synthetic
cgcgccgagggcgggagtttacgcg

SEQ ID NO: 77 - DNA, 21 bp, Artificial Sequence
  synthetic
tggactgagactcctgttctg

SEQ ID NO: 78 - DNA, 23 bp, Artificial Sequence
  synthetic
ctcgacctcactcctattatcgc

SEQ ID NO: 79 - DNA, 30 bp, Artificial Sequence
  synthetic
acggacgcggaggtggccgtcttattcagc

SEQ ID NO: 80 - DNA, 24 bp, Artificial Sequence
  synthetic
cgggatagagtcgattaattaggc

SEQ ID NO: 81 - DNA, 18 bp, Artificial Sequence
  synthetic
taacttcccctcgacccg

SEQ ID NO: 82 - DNA, 24 bp, Artificial Sequence
  synthetic
cgcgccgaggcggttcggtagcgg

SEQ ID NO: 83 - DNA, 24 bp, Artificial Sequence
  synthetic
ttgggtaattattacgtggattcg

SEQ ID NO: 84 - DNA, 16 bp, Artificial Sequence
  synthetic
caactcatccgcgacg

SEQ ID NO: 85 - DNA, 27 bp, Artificial Sequence
  synthetic
aggccacggacggtcgacgcccaacaa

SEQ ID NO: 86 - DNA, 19 bp, Artificial Sequence
  synthetic
gggttaggcgttgggtacg

SEQ ID NO: 87 - DNA, 18 bp, Artificial Sequence
  synthetic
aactcatccgcgacgtcg

SEQ ID NO: 88 - DNA, 29 bp, Artificial Sequence
  synthetic
aggccacggacggacgcccaacaaaaacg

SEQ ID NO: 89 - DNA, 22 bp, Artificial Sequence
  synthetic
gtagcgcgtagggattatgtcg

SEQ ID NO: 90 - DNA, 27 bp, Artificial Sequence
  synthetic
tttccacctaatcctctataaaaccgc

SEQ ID NO: 91 - DNA, 26 bp, Artificial Sequence
  synthetic
aggccacggacgctcgcgatctccgc

SEQ ID NO: 92 - DNA, 19 bp, Artificial Sequence
  synthetic
tggagttatcggaaggcga

SEQ ID NO: 93 - DNA, 18 bp, Artificial Sequence
  synthetic
cgaactcccgaaacgacg

SEQ ID NO: 94 - DNA, 26 bp, Artificial Sequence
  synthetic
cgcgccgagggcgcgaacacaaaacg

SEQ ID NO: 95 - DNA, 22 bp, Artificial Sequence
  synthetic
gttcggcgtagagtttcgtaga

SEQ ID NO: 96 - DNA, 32 bp, Artificial Sequence
  synthetic
gaaaataaaaatttaaaacttttcgaaacgcg

SEQ ID NO: 97 - DNA, 24 bp, Artificial Sequence
  synthetic
cgcgccgagggtaccgcgacgtcc

SEQ ID NO: 98 - DNA, 27 bp, Artificial Sequence
  synthetic
agctcgtccgacagtaccgcgacgtcc

SEQ ID NO: 99 - DNA, 21 bp, Artificial Sequence
  synthetic
ggaggtttcgcgtttcgatta

SEQ ID NO: 100 - DNA, 18 bp, Artificial Sequence
  synthetic
cgaacgatccccgcctac

SEQ ID NO: 101 - DNA, 27 bp, Artificial Sequence
  synthetic
aggccacggacgattcgcgttcgagcg

SEQ ID NO: 102 - DNA, 16 bp, Artificial Sequence
  synthetic
tcgcgtcgtcgttcgt

SEQ ID NO: 103 - DNA, 20 bp, Artificial Sequence
  synthetic
gacgccatctaaaaacgcga

SEQ ID NO: 104 - DNA, 27 bp, Artificial Sequence
  synthetic
cgcgccgaggtcgttcgtgtcggtttc

SEQ ID NO: 105 - DNA, 30 bp, Artificial Sequence
  synthetic
agctcgtccgacatcgttcgtgtcggtttc

SEQ ID NO: 106 - DNA, 29 bp, Artificial Sequence
  synthetic
acggacgcggagtcgttcgtgtcggtttc

SEQ ID NO: 107 - DNA, 29 bp, Artificial Sequence
  synthetic
aggccacggacgtcgttcgtgtcggtttc

SEQ ID NO: 108 - DNA, 23 bp, Artificial Sequence
  synthetic
agagttcggcgtttagagttagc

SEQ ID NO: 109 - DNA, 30 bp, Artificial Sequence
  synthetic
ttaaatataaaatcgaaacctatatccgcg

SEQ ID NO: 110 - DNA, 38 bp, Artificial Sequence
  synthetic
aggccacggacgcggtttttcgaggatttattattaag

SEQ ID NO: 111 - DNA, 23 bp, Artificial Sequence
  synthetic
ggagaagagtgcgtaggttagag

SEQ ID NO: 112 - DNA, 22 bp, Artificial Sequence
  synthetic
catatcgcccgacgtaaaaacc

SEQ ID NO: 113 - DNA, 24 bp, Artificial Sequence
  synthetic
cgcgccgaggctcgcgaaacgccg

SEQ ID NO: 114 - DNA, 27 bp, Artificial Sequence
  synthetic
agctcgtccgacactcgcgaaacgccg

SEQ ID NO: 115 - DNA, 26 bp, Artificial Sequence
  synthetic
acggacgcggagctcgcgaaacgccg

SEQ ID NO: 116 - DNA, 22 bp, Artificial Sequence
  synthetic
agagttggcgagttggttgtac

SEQ ID NO: 117 - DNA, 28 bp, Artificial Sequence
  synthetic
cgaattacaacaaaaccgaataacgcga

SEQ ID NO: 118 - DNA, 26 bp, Artificial Sequence
  synthetic
cgcgccgaggcgatacggaaaggcgt

SEQ ID NO: 119 - DNA, 19 bp, Artificial Sequence
  synthetic
tcggagtcgagtttagcgc

SEQ ID NO: 120 - DNA, 24 bp, Artificial Sequence
  synthetic
cgaaatcgaaaaaacaaaaaccgc

SEQ ID NO: 121 - DNA, 26 bp, Artificial Sequence
  synthetic
aggccacggacgcggcgttcgcgatt

SEQ ID NO: 122 - DNA, 24 bp, Artificial Sequence
  synthetic
cgcgccgaggcggcgttcgcgatt

SEQ ID NO: 123 - DNA, 26 bp, Artificial Sequence
  synthetic
acggacgcggagcggcgttcgcgatt

SEQ ID NO: 124 - DNA, 25 bp, Artificial Sequence
  synthetic
cgcgatagcgttttttattgtcgcg

SEQ ID NO: 125 - DNA, 21 bp, Artificial Sequence
  synthetic
cgcacgaccgtaaaatactcg

SEQ ID NO: 126 - DNA, 30 bp, Artificial Sequence
  synthetic
aggccacggacggtcgaaatcgaaacgctc

SEQ ID NO: 127 - DNA, 185 bp, Homo sapiens
agcctgcctgcgtctcttccttcctccgcgtgggttctagcaacatccactgcagccgggccaggcgagccggcgcgtaccatcggcgcggggggaggagagggccgggcctgggaagatgctgcggaggacgctgcggattcgcgagcccggggtaaggcggcggcgcaccgccccctcccgcc

SEQ ID NO: 128 - DNA, 185 bp, Artificial Sequence
  synthetic
agtttgtttgcgttttttttttttttcgcgtgggttttagtaatatttattgtagtcgggttaggcgagtcggcgcgtattatcggcgcggggggaggagagggtcgggtttgggaagatgttgcggaggacgttgcggattcgcgagttcggggtaaggcggcggcgtatcgttttttttcgtt

SEQ ID NO: 129 - DNA, 416 bp, Homo sapiens
gccggcaaagagctggtgcaggatgcggatcagatcagccagggtccgtggcttcagcacttcggtctgttccatctcgtggggagctggctgcgcgcgcgtctcactgctgggctgcggtggaggagctgagcgagccaaggagctgggggcgagggagcctaacagcccgctagaccgctaagcagacacacacgcacaaacccagcattagagtgccgaaacgtaaggatgtcgtcgcagagacagcaagagacccacccccaggcccctggcagcgcagtggatccgggatcgctggagacgcggtgcacacacaaatcaggttcagatctgtggggttcatcctcccgggccccttttaagcgcttggagtcactaggaatgtaccaacggccctcggagggaggacgagg

SEQ ID NO: 130 - DNA, 416 bp, Artificial Sequence
  synthetic
gtcggtaaagagttggtgtaggatgcggattagattagttagggttcgtggttttagtatttcggtttgttttatttcgtggggagttggttgcgcgcgcgttttattgttgggttgcggtggaggagttgagcgagttaaggagttgggggcgagggagtttaatagttcgttagatcgttaagtagatatatacgtataaatttagtattagagtgtcgaaacgtaaggatgtcgtcgtagagatagtaagagatttatttttaggtttttggtagcgtagtggattcgggatcgttggagacgcggtgtatatataaattaggtttagatttgtggggtttattttttcgggttttttttaagcgtttggagttattaggaatgtattaacggttttcggagggaggacgagg

SEQ ID NO: 131 - DNA, 119 bp, Artificial Sequence
  synthetic
gagtgtcgaaacgtaaggatgtcgtcgtagagatagtaagagatttatttttaggtttttggtagcgtagtggattcgggatcgttggagacgcggtgtatatataaattaggtttaga

SEQ ID NO: 132 - DNA, 21 bp, Artificial Sequence
  synthetic
cgaaacgtaaggatgtcgtcg

SEQ ID NO: 133 - DNA, 27 bp, Artificial Sequence
  synthetic
aatttatatatacaccgcgtctccaac

SEQ ID NO: 134 - DNA, 28 bp, Artificial Sequence
  synthetic
cgcgccgaggcgatcccgaatccactac

SEQ ID NO: 135 - DNA, 155 bp, Homo sapiens
gaggggacctggatccctgaaccccggggcggaaagggagcctccgggcggctgtgggtgccgcgctcctcggagccagcagctgctggggcggcgtccgaactccccaggtctgcgcacggcaatgggggcaccgggccttctgtctgtcctca

SEQ ID NO: 136 - DNA, 155 bp, Artificial Sequence
  synthetic
gaggggatttggatttttgaatttcggggcggaaagggagttttcgggcggttgtgggtgtcgcgtttttcggagttagtagttgttggggcggcgttcgaattttttaggtttgcgtacggtaatgggggtatcgggttttttgtttgttttta

SEQ ID NO: 137 - DNA, 79 bp, Artificial Sequence
  synthetic
gaattcgggtttagcgcgggtttttcgcggtagtggtcgtagttcgggaagttcgggggcgcggtgttttcgtgaattc

SEQ ID NO: 138 - DNA, 18 bp, Artificial Sequence
  synthetic
aaagggagttttcgggcg

SEQ ID NO: 139 - DNA, 19 bp, Artificial Sequence
  synthetic
acccgatacccccattacc

SEQ ID NO: 140 - DNA, 32 bp, Artificial Sequence; identical to SEQ ID NO: 12
  synthetic

SEQ ID NO: 141 - DNA, 97 bp, Artificial Sequence
  synthetic
gcggtggatggggtatagcgcgatttcgtagcggcggcggcggttgtttttattatcgggaggatgttcgggcggatagcgtaggtaattttcgtcg

SEQ ID NO: 142 - DNA, 31 bp, Artificial Sequence; identical to SEQ ID NO: 42
  synthetic

SEQ ID NO: 143 - DNA, 29 bp, Artificial Sequence
  synthetic
cgcgccgaggcgaacatcctcccgataat

SEQ ID NO: 144 - DNA, 77 bp, Artificial Sequence
  synthetic
gtcgtcgttcgggagggtatcggtttcgttcgtttgttcgttcgttcgtttttgttcgttcgtttttcgttcgtcgt

SEQ ID NO: 145 - DNA, 30 bp, Artificial Sequence
  synthetic
aggccacggacgatcggtttcgttcgtttg

SEQ ID NO: 146 - DNA, 28 bp, Artificial Sequence
  synthetic
cgcgccgaggatcggtttcgttcgtttg

SEQ ID NO: 147 - DNA, 103 bp, Homo sapiens
agggcaggggcggggaggctggggggcggcaggagggcccggcgggagcccacgcgggtctgcagcgacaacaggagtgaggtcgagcctggccccacgcgca

SEQ ID NO: 148 - DNA, 103 bp, Artificial Sequence
  synthetic
agggtaggggcggggaggttggggggcggtaggagggttcggcgggagtttacgcgggtttgtagcgataataggagtgaggtcgagtttggttttacgcgta

SEQ ID NO: 149 - DNA, 143 bp, Homo sapiens
ggtggcaacggctggagtgccgtcgcccgcgccactcaccccggcgcggcgccctgcgcggccgctcagcggaaggccagcaggaagatcagtacgacgttgatgagaaccaggagcgccagcacggcggagaccaccacgcg

SEQ ID NO: 150 - DNA, 143 bp, Artificial Sequence
  synthetic
ggtggtaacggttggagtgtcgtcgttcgcgttatttatttcggcgcggcgttttgcgcggtcgtttagcggaaggttagtaggaagattagtacgacgttgatgagaattaggagcgttagtacggcggagattattacgcg

SEQ ID NO: 151 - DNA, 22 bp, Artificial Sequence
  synthetic
cgccgagggcggcgttttgcgc

SEQ ID NO: 152 - DNA, 119 bp, Homo sapiens
gcctttgccccggtttttggcgcgggaggactttcgaccccgacttcggccgctcatggtggcggcggaggcagcttcaaagacacgctgtgaccctgcggctcctgacgccagctctc

SEQ ID NO: 153 - DNA, 153 bp, Artificial Sequence
  synthetic
gtttttgtttcggtttttggcgcgggaggattttcgatttcgatttcggtcgtttatggtggcggcggaggtagttttaaagatacgttgtgattttgcggtttttgacgttagtttttcgccaccataaacgaccggtcgtttatggtggcg

SEQ ID NO: 154 - DNA, 117 bp, Homo sapiens
ccgtgggcgcggacagctgccgggagcggcaggcgtctcgatcggggacgcaggcacttccgtccctgcagagcatcagacgcgtctcgggacactggggacaacatctcctccgcg

SEQ ID NO: 155 - DNA, 117 bp, Artificial Sequence
  synthetic
tcgtgggcgcggatagttgtcgggagcggtaggcgtttcgatcggggacgtaggtattttcgtttttgtagagtattagacgcgtttcgggatattggggataatatttttttcgcg

SEQ ID NO: 156 - DNA, 24 bp, Artificial Sequence
  synthetic
cgcgccgagggcgtttcgatcggg

SEQ ID NO: 157 - DNA, 67 bp, Homo sapiens
acatggactgagactcctgttctgtggccgtcttattcagcttcactgttctggcacctgccactct

SEQ ID NO: 158 - DNA, 139 bp, Homo sapiens
ggccacacaggcccactctggccctctgagcccccggcggacccagggcattcaaggagcggctctgggctgccagcgcaggcctccgcgcaaacacagcaggctggaagtggcgctcatcaccggcacgtcttcccag

SEQ ID NO: 159 - DNA, 139 bp, Artificial Sequence
  synthetic
ggttatataggtttattttggttttttgagttttcggcggatttagggtatttaaggagcggttttgggttgttagcgtaggttttcgcgtaaatatagtaggttggaagtggcgtttattatcggtacgtttttttag

SEQ ID NO: 160 - DNA, 28 bp, Artificial Sequence
  synthetic
ggtttattttggttttttgagttttcgg

SEQ ID NO: 161 - DNA, 24 bp, Artificial Sequence
  synthetic
tccaacctactatatttacgcgaa

SEQ ID NO: 162 - DNA, 35 bp, Artificial Sequence
  synthetic
acggacgcggaggcggatttagggtatttaaggag

SEQ ID NO: 163 - DNA, 33 bp, Artificial Sequence
  synthetic
cgcgccgagggcggatttagggtatttaaggag

SEQ ID NO: 164 - DNA, 118 bp, Homo sapiens
cccgaatggaacgagcagctgagcttcgtggagctcttcccgccgctgacgcgcagcctccgcctgcagctgcgggacgacgcgcccctggtcgacgcggcactcgctacgcacgtgc

SEQ ID NO: 165 - DNA, 118 bp, Artificial Sequence
  synthetic
ttcgaatggaacgagtagttgagtttcgtggagtttttttcgtcgttgacgcgtagttttcgtttgtagttgcgggacgacgcgtttttggtcgacgcggtattcgttacgtacgtgt

SEQ ID NO: 166 - DNA, 95 bp, Homo sapiens
gggccattgccagaagacgtcttctcggggcgccaggattcacctttccttcccgacctcaacttcttcgcggccgactcctgtctccagctatc

SEQ ID NO: 167 - DNA, 95 bp, Artificial Sequence
  synthetic
gggttattgttagaagacgttttttcggggcgttaggatttatttttttttttcgattttaattttttcgcggtcgatttttgtttttagttatt

SEQ ID NO: 168 - DNA, 100 bp, Homo sapiens
ggcccggggccgcctgggcccctaggggctggacgtcaacctgttagatagagggcgtgggaccccccgcaggcggctgctcggacgaccgcatccggag

SEQ ID NO: 169 - DNA, 100 bp, Artificial Sequence
  synthetic
ggttcggggtcgtttgggtttttaggggttggacgttaatttgttagatagagggcgtgggatttttcgtaggcggttgttcggacgatcgtattcggag

SEQ ID NO: 170 - DNA, 90 bp, Homo sapiens
gagcagagggaaggtggtgcgcatggtgggcgagcgcgtgcgcctggaggaccccgattggctgacgtgtaaaccaggacgaggacatga

SEQ ID NO: 171 - DNA, 18 bp, Artificial Sequence
  synthetic
cagagggaaggtggtgcg

SEQ ID NO: 172 - DNA, 20 bp, Artificial Sequence
  synthetic
gtcctcgtcctggtttacac

SEQ ID NO: 173 - DNA, 27 bp, Artificial Sequence
  synthetic
aggccacggacgcgtcagccaatcggg

SEQ ID NO: 174 - DNA, 90 bp, Artificial Sequence
  synthetic
gagtagagggaaggtggtgcgtatggtgggcgagcgcgtgcgtttggaggatttcgattggttgacgtgtaaattaggacgaggatatga

SEQ ID NO: 175 - DNA, 17 bp, Artificial Sequence
  synthetic
tgcgtatggtgggcgag

SEQ ID NO: 176 - DNA, 24 bp, Artificial Sequence
  synthetic
cctaatttacacgtcaaccaatcg

SEQ ID NO: 177 - DNA, 26 bp, Artificial Sequence
  synthetic
aggccacggacggcgcgtgcgtttgg

SEQ ID NO: 178 - DNA, 97 bp, Homo sapiens
gcctgcgtggccacggctaccacccgcacgcgcatccgcaccaccaccaccaccccgtgagtccagccgccgcggccgccgccgccgctgccgcagc

SEQ ID NO: 179 - DNA, 97 bp, Artificial Sequence
  synthetic
gtttgcgtggttacggttattattcgtacgcgtattcgtattattattattatttcgtgagtttagtcgtcgcggtcgtcgtcgtcgttgtcgtagt

SEQ ID NO: 180 - DNA, 120 bp, Homo sapiens
cggtcgggcaggcgggacggagattacctggctgtccaggggaccttatgcagggtttggcccgagcccaggggcagcgaggggcgtctgcggatgcggctccctgtgcggcacaacacc

SEQ ID NO: 181 - DNA, 120 bp, Artificial Sequence
  synthetic
cggtcgggtaggcgggacggagattatttggttgtttaggggattttatgtagggtttggttcgagtttaggggtagcgaggggcgtttgcggatgcggttttttgtgcggtataatatt

SEQ ID NO: 182 - DNA, 108 bp, Homo sapiens
tccggcgccgcgttttctagagaaccgggtctcagcgatgctcatttcagccccgtcttaatgcaacaaacgaaaccccacacgaacgaaaaggaacatgtctgcgct

SEQ ID NO: 183 - DNA, 108 bp, Artificial Sequence
  synthetic
ttcggcgtcgcgttttttagagaatcgggttttagcgatgtttattttagtttcgttttaatgtaataaacgaaattttatacgaacgaaaaggaatatgtttgcgtt

SEQ ID NO: 184 - DNA, 24 bp, Artificial Sequence
  synthetic
cgcaaacatattccttttcgttcg

SEQ ID NO: 185 - DNA, 194 bp, Homo sapiens
ttcctgctctcgcgcgacttcgaagctcaggcggcggcgcaggcgcggtgcacggcgcggggcgggagcctggcgcagccggcagaccgccagcagatggaggcgctcactcggtacctgcgcgcggcgctcgctccctacaactggcccgtgtggctgggcgtgcacgatcggcgcgccgagggcctctacct

SEQ ID NO: 186 - DNA, 194 bp, Artificial Sequence
  synthetic
tttttgttttcgcgcgatttcgaagtttaggcggcggcgtaggcgcggtgtacggcgcggggcgggagtttggcgtagtcggtagatcgttagtagatggaggcgtttattcggtatttgcgcgcggcgttcgttttttataattggttcgtgtggttgggcgtgtacgatcggcgcgtcgagggtttttattt

SEQ ID NO: 187 - DNA, 73 bp, Homo sapiens
gacgcgcagtcgcccccccaggcagcctaggcggcggcagctgctgcggcgactgcaaaggccgatttggagt

SEQ ID NO: 188 - DNA, 73 bp, Artificial Sequence
  synthetic
gacgcgtagtcgtttttttaggtagtttaggcggcggtagttgttgcggcgattgtaaaggtcgatttggagt

SEQ ID NO: 189 - DNA, 141 bp, Homo sapiens
cgccccctcacctccccgatcatgccgttccagacgccatcgatcttctttccgtgcttgccattggtgaccaggtagaggtcgtagctgaagccgatggtatgcgccagccgcttcagaatgtcgatgcagaaacccttg

SEQ ID NO: 190 - DNA, 141 bp, Artificial Sequence
  synthetic
cgtttttttattttttcgattatgtcgttttagacgttatcgatttttttttcgtgtttgttattggtgattaggtagaggtcgtagttgaagtcgatggtatgcgttagtcgttttagaatgtcgatgtagaaatttttg

SEQ ID NO: 191 - DNA, 152 bp, Homo sapiens
gcacccggcgcccgagctgcgaaagggacgcccttctcctcccgcgcggaacttcaggagtgcggggcccgagtgtaaactggaccaccgtggggccgcgcgggcccctgggcatcaccacaaactgtgcctgtggccatcgtgtcaggaca

SEQ ID NO: 192 - DNA, 152 bp, Artificial Sequence
  synthetic
gtattcggcgttcgagttgcgaaagggacgttttttttttttcgcgcggaattttaggagtgcggggttcgagtgtaaattggattatcgtggggtcgcgcgggtttttgggtattattataaattgtgtttgtggttatcgtgttaggata

SEQ ID NO: 193 - DNA, 20 bp, Artificial Sequence
  synthetic
gcgcggaattttaggagtgc

SEQ ID NO: 194 - DNA, 18 bp, Artificial Sequence
  synthetic
cgcgaccccacgataatc

SEQ ID NO: 195 - DNA, 30 bp, Artificial Sequence; identical to SEQ ID NO: 9
  synthetic

SEQ ID NO: 196 - DNA, 111 bp, Homo sapiens
aggctccgcgtcgcggtccccgctcgccctcccgcccgcccaccgggcaccccagccgcgcagaaggcggaagccacgcgcgagggaccgcggtccgtccgggactagccc

SEQ ID NO: 197 - DNA, 110 bp, Artificial Sequence
  synthetic
aggtttcgcgtcgcggttttcgttcgttttttcgttcgtttatcgggtattttagtcgcgtagaaggcggaagttacgcgcgagggatcgcggttcgttcgggattagtt

SEQ ID NO: 198 - DNA, 92 bp, Homo sapiens
ggccgcgacccctccccgctgacctcactcgagccgccgcctggcgcagatataagcggcggcccatctgaagagggctcggcaggcgcccg

SEQ ID NO: 199 - DNA, 92 bp, Artificial Sequence
  synthetic
ggttgtgatttttttttgttgattttatttgagttgttgtttggtgtagatataagtggtggtttatttgaagagggtttggtaggtgtttg

SEQ ID NO: 200 - DNA, 92 bp, Artificial Sequence
  synthetic
ggtcgcgattttttttcgttgattttattcgagtcgtcgtttggcgtagatataagcggcggtttatttgaagagggttcggtaggcgttcg

SEQ ID NO: 201 - DNA, 97 bp, Homo sapiens
aggccccgcccagcgctgccagctgctttacatatcggcgcccgggctaccgcgggcctcgcggctaagcgtgcacagccgcagctctgcagcgccg

SEQ ID NO: 202 - DNA, 97 bp, Artificial Sequence
  synthetic
aggtttcgtttagcgttgttagttgttttatatatcggcgttcgggttatcgcgggtttcgcggttaagcgtgtatagtcgtagttttgtagcgtcg

SEQ ID NO: 203 - DNA, 95 bp, Homo sapiens
gatgtcatgggccagtgggacccgccgttcaactgcagctcgggcgacttcatcttctgctgcgggacttgtggcttccggttctgctgcacgtt

SEQ ID NO: 204 - DNA, 95 bp, Artificial Sequence
  synthetic
gatgttatgggttagtgggattcgtcgtttaattgtagttcgggcgattttattttttgttgcgggatttgtggttttcggttttgttgtacgtt

SEQ ID NO: 205 - DNA, 120 bp, Homo sapiens
aagctgcgcccggagacgtgggagcgttctcttgttttccgagtgcgcggactcatcgggtcacagtttatgcttttatgacgcggtgagtccagccactgattcctaacggtttagagt

SEQ ID NO: 206 - DNA, 120 bp, Artificial Sequence
  synthetic
aagttgcgttcggagacgtgggagcgtttttttgtttttcgagtgcgcggatttatcgggttatagtttatgtttttatgacgcggtgagtttagttattgatttttaacggtttagagt

SEQ ID NO: 207 - DNA, 133 bp, Homo sapiens
ggtcgccctcggcgagggtccgggagaaggctcggctccctcctaaacatgtggcccgtggcgtccccttgtcccctccgagcgatgctcctgcgcccttcgccgcctcccgcgctgctgcgccgccaggcaa

SEQ ID NO: 208 - DNA, 123 bp, Artificial Sequence
  synthetic
ggcgagggttcgggagaaggttcggtttttttttaaatatgtggttcgtggcgtttttttgtttttttcgagcgatgtttttgcgtttttcgtcgtttttcgcgttgttgcgtcgttaggtaa

SEQ ID NO: 209 - DNA, 101 bp, Homo sapiens
gggcgggccaggcgctgggcacggtgatggccaccactggggccctgggcaactactacgtggactcgttcctgctgggcgccgacgccgcggatgagctg

SEQ ID NO: 210 - DNA, 101 bp, Artificial Sequence
  synthetic
gggcgggttaggcgttgggtacggtgatggttattattggggttttgggtaattattacgtggattcgtttttgttgggcgtcgacgtcgcggatgagttg

SEQ ID NO: 211 - DNA, 101 bp, Artificial Sequence; identical to SEQ ID NO: 210
  synthetic

SEQ ID NO: 212 - DNA, 101 bp, Artificial Sequence; identical to SEQ ID NO: 210
  synthetic

SEQ ID NO: 213 - DNA, 160 bp, Homo sapiens
gccgcttctccctgccccgcagcgcgcagggaccatgtcggcggagaccgcgagcggccccacagaggaccaggtggaaatcctggagtacaacttcaacaaggtcgacaagcacccggattccaccacgctgtgcctcatcgcggccgaggcaggcctt

SEQ ID NO: 214 - DNA, 161 bp, Artificial Sequence
  synthetic
cgtcgtttttttttgtttcgtagcgcgtagggattatgtcggcggagatcgcgagcggttttatagaggattaggtggaaattttggagtataattttaataaggtcgataagtattcggattttattacgttgtgttttatcgcggtcgaggtaggtttt

SEQ ID NO: 215 - DNA, 72 bp, Homo sapiens
cgggacagagccgaccaatcaggcggctcggcagcggggcagaggtcagggggcgggccgaggggaagccaa

SEQ ID NO: 216 - DNA, 72 bp, Artificial Sequence
  synthetic
cgggatagagtcgattaattaggcggttcggtagcggggtagaggttagggggcgggtcgaggggaagttaa

SEQ ID NO: 217 - DNA, 290 bp, Homo sapiens
tcggcagaagcagcaaaccgccggcaagcccagcgaggagggctgccggggtctgggcttgggaattggctggcacccagcggaaagggacgtgagctgagcggcgggggagaagagtgcgcaggtcagagggcggcgcgcagcggcgctccgcgaggtccccacgccgggcgatatggggtgcctgctgtttctgctgctctgggcgctcctccaggcttggggaagcgctgaaggtgggtggaacgagggcgcttgagtgcactcgcgggagggcggagagagggagc

SEQ ID NO: 218 - DNA, 290 bp, Artificial Sequence
  synthetic
tcggtagaagtagtaaatcgtcggtaagtttagcgaggagggttgtcggggtttgggtttgggaattggttggtatttagcggaaagggacgtgagttgagcggcgggggagaagagtgcgtaggttagagggcggcgcgtagcggcgtttcgcgaggtttttacgtcgggcgatatggggtgtttgttgtttttgttgttttgggcgtttttttaggtttggggaagcgttgaaggtgggtggaacgagggcgtttgagtgtattcgcgggagggcggagagagggagt

SEQ ID NO: 219 - DNA, 134 bp, Homo sapiens
gcgggatcatgggcgttagcctggctctctccgacgaggcaaactgcaggagatccccttccctggagtcaccggaaggcgaaagcacgctctgtgcccgcgcgccgccccgggagcccgctcctaccccctcg

SEQ ID NO: 220 - DNA, 134 bp, Artificial Sequence
  synthetic
gcgggattatgggcgttagtttggtttttttcgacgaggtaaattgtaggagatttttttttttggagttatcggaaggcgaaagtacgttttgtgttcgcgcgtcgtttcgggagttcgtttttattttttcg

SEQ ID NO: 221 - DNA, 85 bp, Artificial Sequence
  synthetic
gtcgagggcgttcggcgtagagtttcgtagaggcggacgtcgcggtacgcgtttcgaaaagttttaaatttttattttcggtttt

SEQ ID NO: 222 - DNA, 172 bp, Homo sapiens
tgcggggctgcttccccgcgtcctccgggcccgggccgccctcctcccgcacagtgcggagcagggaggccccgcgcctcgaccacccgcgcccgagcgtccgcgcctcctcctccgctctgcaggcggggaccgcccggcgctcggcacccggcagcgcggccccctccag

SEQ ID NO: 223 - DNA, 172 bp, Artificial Sequence
  synthetic
tgcggggttgttttttcgcgtttttcgggttcgggtcgtttttttttcgtatagtgcggagtagggaggtttcgcgtttcgattattcgcgttcgagcgttcgcgttttttttttcgttttgtaggcggggatcgttcggcgttcggtattcggtagcgcggttttttttag

SEQ ID NO: 224 - DNA, 101 bp, Homo sapiens
gccaaggggcgcccgcgccgccgcccgccgcccgtgccggccccggccgttcgggctcggtgcgcaccgcgcccccagatggcgccgccaagcgtccgccc

SEQ ID NO: 225 - DNA, 101 bp, Artificial Sequence
  synthetic
gttaaggggcgttcgcgtcgtcgttcgtcgttcgtgtcggtttcggtcgttcgggttcggtgcgtatcgcgtttttagatggcgtcgttaagcgttcgttt

SEQ ID NO: 226 - DNA, 375 bp, Homo sapiens
ccttcacacgccactcccactgcctcagggaaccacaagcacaggcatcgccccgcccggcgtcctcctccaaagattggggcaccaggaccgcgggcccccactcccacccagcacaaagagtccggcgctcagagctagcggtttcccgaggactcaccaccaagcccgcggacacaggccccgattccacacttaacgctgccaaagtggcagagccggcgcgggctgggacagaggcggcactgaggccggcgctgtcggtggctgagagcgccacaagtctcggtccgttacaccaggggcgacgcttcccagaggcccccgcggctcacccgggcgggactggcttcactgtttgcgcgtcctgagaag

SEQ ID NO: 227 - DNA, 375 bp, Artificial Sequence
  synthetic
tttttatacgttatttttattgttttagggaattataagtataggtatcgtttcgttcggcgtttttttttaaagattggggtattaggatcgcgggtttttatttttatttagtataaagagttcggcgtttagagttagcggtttttcgaggatttattattaagttcgcggatataggtttcgattttatatttaacgttgttaaagtggtagagtcggcgcgggttgggatagaggcggtattgaggtcggcgttgtcggtggttgagagcgttataagtttcggttcgttatattaggggcgacgttttttagaggttttcgcggtttattcgggcgggattggttttattgtttgcgcgttttgagaag

SEQ ID NO: 228 - DNA, 105 bp, Artificial Sequence
  synthetic
tttatttagtataaagagttcggcgtttagagttagcggtttttcgaggatttattattaagttcgcggatataggtttcgattttatatttaacgttgttaaag

SEQ ID NO: 229 - DNA, 95 bp, Homo sapiens; identical to SEQ ID NO: 203

SEQ ID NO: 230 - DNA, 95 bp, Artificial Sequence; identical to SEQ ID NO: 204
  synthetic

SEQ ID NO: 231 - DNA, 124 bp, Homo sapiens
ggagagttggcgagctggctgcacgacacggaaaggcgctctcctttccactttttggccctcgcgctacccggttttgctgcaatccggaccgcggtaggaagtgaaatgaacgtggcggagg

SEQ ID NO: 232 - DNA, 124 bp, Artificial Sequence
  synthetic
ggagagttggcgagttggttgtacgatacggaaaggcgtttttttttttattttttggttttcgcgttattcggttttgttgtaattcggatcgcggtaggaagtgaaatgaacgtggcggagg

SEQ ID NO: 233 - DNA, 72 bp, Artificial Sequence
  synthetic
gggatcggagtcgagtttagcgcggcgttcgcgattcgttagtcgcggtttttgttttttcgatttcgcgcg

SEQ ID NO: 234 - DNA, 274 bp, Homo sapiens
gcctgctggccggggacccgcgcgtcgagcgcctggtgcgcgacagcgcctcctactgccgcgagcgcttcgaccccgacgagtactccacggccgtgcgcgaggcgccagcggagctcgccgaagactgcgccagcccgcgccgcgcgcgcctctgcctgcccgcgccgcgcgcccccgggctcgcccgcgcccccggcccgctagcgccggctcccgccggcgagggcgaccaggagtacgtgagccccgactgggcagccgcgcccgagcc

SEQ ID NO: 235 - DNA, 274 bp, Artificial Sequence
  synthetic
gtttgttggtcggggattcgcgcgtcgagcgtttggtgcgcgatagcgttttttattgtcgcgagcgtttcgatttcgacgagtattttacggtcgtgcgcgaggcgttagcggagttcgtcgaagattgcgttagttcgcgtcgcgcgcgtttttgtttgttcgcgtcgcgcgttttcgggttcgttcgcgttttcggttcgttagcgtcggttttcgtcggcgagggcgattaggagtacgtgagtttcgattgggtagtcgcgttcgagtt

SEQ ID NO: 236 - DNA, 128 bp, Artificial Sequence
  synthetic
gggtttgttggtcggggattcgcgcgtcgagcgtttggtgcgcgatagcgttttttattgtcgcgagcgtttcgatttcgacgagtattttacggtcgtgcgcgaggcgttagcggagttcgtcgaag